SEQUENCE LISTING (881 sequences)

SEQ ID NO: 1 - protein, 25 aa, Homo sapiens
PEDAELVRLSKRLVENAVLKAVQQY

SEQ ID NO: 2 - protein, 25 aa, Homo sapiens
PEDAELVRTSKRLVENAVLKAVQQY

SEQ ID NO: 3 - protein, 25 aa, Homo sapiens
PEDAELVRLSKRDVENAVLKAVQQY

SEQ ID NO: 4 - protein, 25 aa, Homo sapiens
PEDAELVRLSKRLVENAVEKAVQQY

SEQ ID NO: 5 - protein, 25 aa, Homo sapiens
PEDAELVRLSKRLPENAVLKAVQQY

SEQ ID NO: 6 - protein, 25 aa, Homo sapiens
PEDAELVRLSKRLPENAPLKAVQQY

SEQ ID NO: 7 - protein, 25 aa, Homo sapiens
PEDAELVRLDKRLPENAPLKAVQQY

SEQ ID NO: 8 - protein, 31 aa, Homo sapiens
EPEDAELVRLSKRLVENAVLKAVQQYLEETQ

SEQ ID NO: 9 - protein, 25 aa, Homo sapiens
NTDEAQEELAWKIAKMIVSDIMQQA

SEQ ID NO: 10 - protein, 25 aa, Homo sapiens
VNLDKKAVLAEKIVAEAIEKAEREL

SEQ ID NO: 11 - protein, 25 aa, Homo sapiens
NGILELETKSSKLVQNIIQTAVDQF

SEQ ID NO: 12 - protein, 25 aa, Homo sapiens
TQDKNYEDELTQVALALVEDVINYA

SEQ ID NO: 13 - protein, 25 aa, Homo sapiens
LVDDPLEYQAGLLVQNAIQQAIAEQ

SEQ ID NO: 14 - protein, 25 aa, Homo sapiens
QYETLLIETASSLVKNAIQLSIEQL

SEQ ID NO: 15 - protein, 25 aa, Homo sapiens
LEKQYQEQLEEEVAKVIVSMSIAFA

SEQ ID NO: 16 - protein, 25 aa, Homo sapiens
EEGLDRNEEIKRAAFQIISQVISEA

SEQ ID NO: 17 - protein, 25 aa, Homo sapiens
ETSAKDNINIEEAARFLVEKILVNH

SEQ ID NO: 18 - protein, 25 aa, Homo sapiens
ADRGSPALSSEALVRVLVLDANDNS

SEQ ID NO: 19 - protein, 25 aa, Homo sapiens
SDRGSPALSSEALVRVLVLDANDNS

SEQ ID NO: 20 - protein, 25 aa, Homo sapiens
TDRGFPALSSEALVRVLVLDANDNS

SEQ ID NO: 21 - protein, 25 aa, Homo sapiens
FLAGETESLADIVLWGALYPLLQDP

SEQ ID NO: 22 - protein, 25 aa, Homo sapiens
SELLKQVSAAASVVSQALHDLLQHV

SEQ ID NO: 23 - protein, 25 aa, Homo sapiens
EKESLTEEEATEFLKQILNGVYYLH

SEQ ID NO: 24 - protein, 25 aa, Homo sapiens
EKGYYSERDAADAVKQILEAVAYLH

SEQ ID NO: 25 - protein, 25 aa, Homo sapiens
WLYLQDQNKAADAVGEILLSLSYLP

SEQ ID NO: 26 - protein, 25 aa, Homo sapiens
LKISPVAPDADAVAAQILSLLPLKF

SEQ ID NO: 27 - protein, 25 aa, Homo sapiens
SKTEQPAALALDLVNKLVYWVDLYL

SEQ ID NO: 28 - protein, 25 aa, Homo sapiens
VLASAYTGRLSMAAADIVNFLTVGS

SEQ ID NO: 29 - protein, 25 aa, Homo sapiens
VKLSNLSNLSHDLVQEAIDHAQDLQ

SEQ ID NO: 30 - protein, 25 aa, Homo sapiens
APSDPDAVSAEEALKYLLHLVDVNE

SEQ ID NO: 31 - protein, 25 aa, Homo sapiens
QMKAKRTKEAVEVLKKALDAISHSD

SEQ ID NO: 32 - protein, 25 aa, Homo sapiens
KDKLKPGAAEDDLVLEVVIMIGTVS

SEQ ID NO: 33 - protein, 25 aa, Homo sapiens
EKRVADPTLEKYVLSVVLDTINAFF

SEQ ID NO: 34 - protein, 25 aa, Homo sapiens
QENLSLIGVANVFLESLFYDVKLQY

SEQ ID NO: 35 - protein, 25 aa, Homo sapiens
HQSVVYRKQAAMILNELVTGAAGLE

SEQ ID NO: 36 - protein, 25 aa, Homo sapiens
QQLQKQLKEAEQILATAVYQAKEKL

SEQ ID NO: 37 - protein, 25 aa, Homo sapiens
HSVMDTLAVALRVAEEAIEEAISKA

SEQ ID NO: 38 - protein, 25 aa, Homo sapiens
RQVQETLNLEPDVAQHLLAHSHWGA

SEQ ID NO: 39 - protein, 25 aa, Homo sapiens
DIPSADRHKSKLIAGKIIPAIATTT

SEQ ID NO: 40 - protein, 25 aa, Homo sapiens
ANDAQLVRLSKRLVENAVLKAVQQY

SEQ ID NO: 41 - protein, 25 aa, Homo sapiens
ANDAQLVRLSKRLVENAVLKAVQQY

SEQ ID NO: 42 - protein, 25 aa, Homo sapiens
ASDAQLVRLSKRLVENAVLKAVQQY

SEQ ID NO: 43 - protein, 25 aa, Homo sapiens
ASDAKLVRLSKRLVENAVLKAVQQY

SEQ ID NO: 44 - protein, 25 aa, Homo sapiens
ARDAKLVRLSKRLVENAVLKAVQQY

SEQ ID NO: 45 - protein, 25 aa, Homo sapiens
ARDAQLVRLSKRLVENAVLKAVQQY

SEQ ID NO: 46 - protein, 25 aa, Homo sapiens
ANDARLVRLSKRLVENAVLKAVQQY

SEQ ID NO: 47 - protein, 25 aa, Homo sapiens
ASDARLVRLSKRLVENAVLKAVQQY

SEQ ID NO: 48 - protein, 25 aa, Homo sapiens
ASDAKTVRLSKRLVENAVLKAVQQY

SEQ ID NO: 49 - protein, 25 aa, Homo sapiens
ANDAKTERLSKRLVENAVLKAVQQY

SEQ ID NO: 50 - protein, 25 aa, Homo sapiens
ANDAKTERLSQRLVENAVLKAVQQY

SEQ ID NO: 51 - protein, 25 aa, Homo sapiens
ANDAKTQRLSQRLVENAVLKAVQQY

SEQ ID NO: 52 - protein, 25 aa, Homo sapiens
PEDAELVRLSKRLVENAVLKAVQQY

SEQ ID NO: 53 - protein, 25 aa, Homo sapiens
PEDAELVRLSKRLVENAVLQAVQQY

SEQ ID NO: 54 - protein, 25 aa, Homo sapiens
PEDAELVRLSKRLVENAVLNGVQQY

SEQ ID NO: 55 - protein, 25 aa, Homo sapiens
PEDAELVRLSKRLVENAVLNGQQQY

SEQ ID NO: 56 - protein, 25 aa, Homo sapiens
PEDAELVRLSKRLVENAVLNGNQQY

SEQ ID NO: 57 - protein, 25 aa, Homo sapiens
PEDAELVRLSKRLVENAVKNGNQQY

SEQ ID NO: 58 - protein, 25 aa, Homo sapiens
PEDAELVRLSKRLVENAVKNGAQDY

SEQ ID NO: 59 - protein, 25 aa, Homo sapiens
ANDARLVRLSKRLRENAVLKAVQQY

SEQ ID NO: 60 - protein, 25 aa, Homo sapiens
ANDARLVRLSKRLYENAVLKAVQQY

SEQ ID NO: 61 - protein, 25 aa, Homo sapiens
ANDARLVRLSKRLVENAVLKFVQQY

SEQ ID NO: 62 - protein, 25 aa, Homo sapiens
ANDARLVRLNKRLVENAVLKAVQQY

SEQ ID NO: 63 - protein, 25 aa, Homo sapiens
ANDARLVRLPKRLVENAVLKAVQQY

SEQ ID NO: 64 - protein, 25 aa, Homo sapiens
ANDARLVRLSKRDVENAVLKAVQQY

SEQ ID NO: 65 - protein, 25 aa, Homo sapiens
YQEQLEEEVAKVIVSMSIAFAQQTE

SEQ ID NO: 66 - protein, 25 aa, Homo sapiens
NLQKIVEEKVAAALVSQIQLEAVQE

SEQ ID NO: 67 - protein, 25 aa, Homo sapiens
YIALNEDLRSWTAADTAAQISQRKL

SEQ ID NO: 68 - protein, 25 aa, Homo sapiens
ISKEHWNPTIVALVYNVLKTLMEMN

SEQ ID NO: 69 - protein, 25 aa, Homo sapiens
DAPEFHSRYITTVIQRIFYTVNRSW

SEQ ID NO: 70 - protein, 25 aa, Homo sapiens
GSTFQNTYNLKDIAGEAISFASGKI

SEQ ID NO: 71 - protein, 25 aa, Homo sapiens
LSRYNDQASSSRIIERIFSGAVTRG

SEQ ID NO: 72 - protein, 25 aa, Homo sapiens
EASEFHSRYITTVIQRIFYAVNRSW

SEQ ID NO: 73 - protein, 25 aa, Homo sapiens
QSEYSWRKMLSGIAAFLLGLIFLLV

SEQ ID NO: 74 - protein, 25 aa, Homo sapiens
LLLLFWLGWLGMLAGAVVIIVRAPR

SEQ ID NO: 75 - protein, 25 aa, Homo sapiens
LYFHLAPKAEVGVIAKALVRLLRSH

SEQ ID NO: 76 - protein, 25 aa, Homo sapiens
VKQNVKMSESQAALPSALKTLQQKL

SEQ ID NO: 77 - protein, 25 aa, Homo sapiens
DPGALGRLGAWALLFFLVTTLLASA

SEQ ID NO: 78 - protein, 25 aa, Homo sapiens
GQEVEGMNILGLVVFAIVFGVALRK

SEQ ID NO: 79 - protein, 25 aa, Homo sapiens
EPTTGMDPKARRFLWNLILDLIKTG

SEQ ID NO: 80 - protein, 25 aa, Homo sapiens
RLRGISWKDEARVVKWALEKLELTK

SEQ ID NO: 81 - protein, 25 aa, Homo sapiens
LIQSLESNPLTKIAWRAAKPLLMGK

SEQ ID NO: 82 - protein, 25 aa, Homo sapiens
AYTTQSLASVAYLINTLANNVLQML

SEQ ID NO: 83 - protein, 25 aa, Homo sapiens
TLSKTKNLRLLILVGRLFMWEEPEI

SEQ ID NO: 84 - protein, 25 aa, Homo sapiens
RYGAAEPHTIAAFLGGAAAQEVIKI

SEQ ID NO: 85 - protein, 25 aa, Homo sapiens
NKDDDESPGLYGFLHVIVHSAKGFK

SEQ ID NO: 86 - protein, 25 aa, Homo sapiens
PTTHAMSPRLRHVLLELLPRLLGSP

SEQ ID NO: 87 - protein, 25 aa, Homo sapiens
PGSAQEKSIERRFLNGLFSKLQPRD

SEQ ID NO: 88 - protein, 25 aa, Homo sapiens
HFDPTTAFRAPDVARALLRQIQVSR

SEQ ID NO: 89 - protein, 25 aa, Homo sapiens
HYKETWKALEALVAKGLVQALGLSN

SEQ ID NO: 90 - protein, 25 aa, Homo sapiens
QKLYLDRNLIAAVAPGAFLGLKALR

SEQ ID NO: 91 - protein, 25 aa, Homo sapiens
KEIKSYLKRIFQLVRFLFPELPEEG

SEQ ID NO: 92 - protein, 25 aa, Homo sapiens
AISPWKTYQLVYFLDKILQKSPLPP

SEQ ID NO: 93 - protein, 25 aa, Homo sapiens
GGAAGDEAREAAAVRALVARLLGPG

SEQ ID NO: 94 - protein, 25 aa, Homo sapiens
GARSGHEQVVEMLLDRAAPILSKTK

SEQ ID NO: 95 - protein, 25 aa, Homo sapiens
SRTSSPVKSSLFLAPSALKLSTPSS

SEQ ID NO: 96 - protein, 25 aa, Homo sapiens
TSYPWSWARVGPAVELALAQVKARP

SEQ ID NO: 97 - protein, 25 aa, Homo sapiens
TIDRETSGNLEQLLLAVVKSIRSIP

SEQ ID NO: 98 - protein, 25 aa, Homo sapiens
AVQNRFHGDAQVALLGLASVIKNTP

SEQ ID NO: 99 - protein, 25 aa, Homo sapiens
PFRIYQTTTERPFIQKLFRPVAADG

SEQ ID NO: 100 - protein, 25 aa, Homo sapiens
RDRASYEARERHVAERLLMHLEEMQ

SEQ ID NO: 101 - protein, 25 aa, Homo sapiens
EKLKSRPAHLGVFLRYIFSQADPSP

SEQ ID NO: 102 - protein, 25 aa, Homo sapiens
NNTEKTVKKIKAFVEQVANVVLYSS

SEQ ID NO: 103 - protein, 25 aa, Homo sapiens
EINVERDEKLIKVLDKLLLYLRIVH

SEQ ID NO: 104 - protein, 25 aa, Homo sapiens
ELLQSETDKVVRAVAIALRNLSLDR

SEQ ID NO: 105 - protein, 25 aa, Homo sapiens
LVASSQSVREAKAASHVLQTVWSYK

SEQ ID NO: 106 - protein, 25 aa, Homo sapiens
EFKTLSEEEIEKVLKNIFNISLQRK

SEQ ID NO: 107 - protein, 25 aa, Homo sapiens
EFKTLTEEEVEEVLKNIFKISLGRN

SEQ ID NO: 108 - protein, 25 aa, Homo sapiens
VINLIGQTPISRLVALLVRGLGTEK

SEQ ID NO: 109 - protein, 25 aa, Homo sapiens
GSAAMAASSVSVVLSSLFLKLYRKP

SEQ ID NO: 110 - protein, 25 aa, Homo sapiens
PRSVRDTILSRALILKILMSAAIII

SEQ ID NO: 111 - protein, 25 aa, Homo sapiens
EWTGYTAFFVGILVQQIADLIIRKT

SEQ ID NO: 112 - protein, 25 aa, Homo sapiens
ADDQGQHPLLKMLLHLLAFSSAATG

SEQ ID NO: 113 - protein, 25 aa, Homo sapiens
GLGGAWAFVLRDVIYTLIHYINQRP

SEQ ID NO: 114 - protein, 25 aa, Homo sapiens
STDEYYYYALAIVVMSIVSIVSSLY

SEQ ID NO: 115 - protein, 25 aa, Homo sapiens
APQRKTTNPLDLAVMRLAALEQNVE

SEQ ID NO: 116 - protein, 25 aa, Homo sapiens
MGALARALLLPLLAQWLLRAAPELA

SEQ ID NO: 117 - protein, 25 aa, Homo sapiens
TTLLRLPQKVFDAVVEAVARASLIP

SEQ ID NO: 118 - protein, 25 aa, Homo sapiens
DASYSLILEVQTAIDNVLIQSDVPI

SEQ ID NO: 119 - protein, 25 aa, Homo sapiens
EKEEVPEGMEEAAVASVVLPARELQ

SEQ ID NO: 120 - protein, 25 aa, Homo sapiens
SHELRSKILSLQLLLSILQNAGPIF

SEQ ID NO: 121 - protein, 25 aa, Homo sapiens
SHREAWTNLLLLFLTKVLKISDNRF

SEQ ID NO: 122 - protein, 25 aa, Homo sapiens
HQSNKGYSLASLLAKVAAGKEKSSN

SEQ ID NO: 123 - protein, 25 aa, Homo sapiens
LIQTSSTEQLRTIIRYLLDTLLSLL

SEQ ID NO: 124 - protein, 25 aa, Homo sapiens
RGNLPTSGNISGFIRRLFLQLMLED

SEQ ID NO: 125 - protein, 25 aa, Homo sapiens
VTFHLPHHVLKSIASAIVNELKKIN

SEQ ID NO: 126 - protein, 25 aa, Homo sapiens
GAVYKGSLDERPVAVKVFSFANRQN

SEQ ID NO: 127 - protein, 25 aa, Homo sapiens
LTKISLKNNLDHLLASLLFEEYISY

SEQ ID NO: 128 - protein, 25 aa, Homo sapiens
LVQLVDTLGAEKFLWILLILLFEQY

SEQ ID NO: 129 - protein, 25 aa, Homo sapiens
RLTSLKKTLATTLAPRVLLPAIKKT

SEQ ID NO: 130 - protein, 25 aa, Homo sapiens
PISGQTYSVEDAVLKGVVDPEFRIR

SEQ ID NO: 131 - protein, 25 aa, Homo sapiens
WAKQHQQRLASALAGLIAKQELLEA

SEQ ID NO: 132 - protein, 25 aa, Homo sapiens
HKAELKPLRADYLIARVVTVLEKLI

SEQ ID NO: 133 - protein, 25 aa, Homo sapiens
ASTLILTPTSKDVLSNLVMISRGKE

SEQ ID NO: 134 - protein, 25 aa, Homo sapiens
ENREKVNDQAKLIVGIVVGLLLAAL

SEQ ID NO: 135 - protein, 25 aa, Homo sapiens
YRNGKVLHPLEGAVVIIFKKEMDPV

SEQ ID NO: 136 - protein, 25 aa, Homo sapiens
NITDLGAYKLAEALPSLAASLLRLS

SEQ ID NO: 137 - protein, 25 aa, Homo sapiens
DFRKKARQSIQGILEAAFSEELTRS

SEQ ID NO: 138 - protein, 25 aa, Homo sapiens
SIGLQNFEIAKDFVVKVIDRLSRDE

SEQ ID NO: 139 - protein, 25 aa, Homo sapiens
VKPSSTRGGVLFAITDAFQKVIYLG

SEQ ID NO: 140 - protein, 25 aa, Homo sapiens
RLGEQNFHKARRFVEQVARRLTLAR

SEQ ID NO: 141 - protein, 25 aa, Homo sapiens
SIGYTNFTLEKNFVINVVNRLGAIA

SEQ ID NO: 142 - protein, 25 aa, Homo sapiens
IYDERGARQLGLALGPALGLLGDPF

SEQ ID NO: 143 - protein, 25 aa, Homo sapiens
GSFAWHMNRSIVLLLKVLAQLRDHS

SEQ ID NO: 144 - protein, 25 aa, Homo sapiens
YKTSTVSADLANLLKRIATIVPRTE

SEQ ID NO: 145 - protein, 25 aa, Homo sapiens
HSGFIETEELKNFLKDLLEKANKTV

SEQ ID NO: 146 - protein, 25 aa, Homo sapiens
TNTQVEGDDEAAFLERLARREERRQ

SEQ ID NO: 147 - protein, 25 aa, Homo sapiens
FQLQRPPQNLLRLLRKAVERSSLMG

SEQ ID NO: 148 - protein, 25 aa, Homo sapiens
LYLRQNSMGLFSALRHALAKESLVG

SEQ ID NO: 149 - protein, 25 aa, Homo sapiens
DKVTQKQFQLKEIVLELVAQVLEHK

SEQ ID NO: 150 - protein, 25 aa, Homo sapiens
EGFHLLGQPLSHLARRLLDTVWNKG

SEQ ID NO: 151 - protein, 25 aa, Homo sapiens
ELYLRKRHHEPGVADRLIRLLQETS

SEQ ID NO: 152 - protein, 25 aa, Homo sapiens
LIRSLYEMQEERLARKAARGLNVGH

SEQ ID NO: 153 - protein, 25 aa, Homo sapiens
RLLDLATVKANGLAAFLLQHVQELP

SEQ ID NO: 154 - protein, 25 aa, Homo sapiens
EGFSFNPLKIEVFVQTLLHLAAKSF

SEQ ID NO: 155 - protein, 25 aa, Homo sapiens
RITQDAQLKSSKVVHKAVLQLNEEG

SEQ ID NO: 156 - protein, 25 aa, Homo sapiens
VKKAPDAEELDKVARLAAKALASVS

SEQ ID NO: 157 - protein, 25 aa, Homo sapiens
MEQVAHQTIVMQFILELAKSLKVDP

SEQ ID NO: 158 - protein, 25 aa, Homo sapiens
FKITRYWNSLSNLVASLLNSVRSIA

SEQ ID NO: 159 - protein, 25 aa, Homo sapiens
HIPTPGAALSWQAAIDAARQAKLMG

SEQ ID NO: 160 - protein, 25 aa, Homo sapiens
NWLTEVQDTANKALLALFTAEMLLK

SEQ ID NO: 161 - protein, 25 aa, Homo sapiens
PNSSKQTVLSWQAAIDAARQAKAAQ

SEQ ID NO: 162 - protein, 25 aa, Homo sapiens
RTLLWTFIKSFQALPYVALLIAMIF

SEQ ID NO: 163 - protein, 25 aa, Homo sapiens
VRHKYNFDNLGQALMSLFVLASKDG

SEQ ID NO: 164 - protein, 25 aa, Homo sapiens
LRVISRAPGLKLVVETLISSLKPIG

SEQ ID NO: 165 - protein, 25 aa, Homo sapiens
NYMFTTVFVLEAVLKLVAFGLRRFF

SEQ ID NO: 166 - protein, 25 aa, Homo sapiens
VGNLGLLFMLLFFIYAALGVELFGK

SEQ ID NO: 167 - protein, 25 aa, Homo sapiens
VHHKYNFDNLGQALMSLFVLASKDG

SEQ ID NO: 168 - protein, 25 aa, Homo sapiens
FKITKYWTSLSNLVASLLNSIRSIA

SEQ ID NO: 169 - protein, 25 aa, Homo sapiens
HNQPLWLTRLQDIANRVLLSLFTTE

SEQ ID NO: 170 - protein, 25 aa, Homo sapiens
VKSKEEAQHVQRVLAQLLRREAALT

SEQ ID NO: 171 - protein, 25 aa, Homo sapiens
RVAQDLGLELAELVPRLFRVASKTH

SEQ ID NO: 172 - protein, 25 aa, Homo sapiens
VFSRRGGLGARDLLLWLLLLAAWEV

SEQ ID NO: 173 - protein, 25 aa, Homo sapiens
RIAQDLGLELEELVPRLFRVASKRH

SEQ ID NO: 174 - protein, 25 aa, Homo sapiens
RRGRGAWTRLLSLLLLAAWEVGSGQ

SEQ ID NO: 175 - protein, 25 aa, Homo sapiens
RIAQDLGLELAELVPRLFRVASKRH

SEQ ID NO: 176 - protein, 25 aa, Homo sapiens
RIAQDLGLELAELVPRLFRVASKGR

SEQ ID NO: 177 - protein, 25 aa, Homo sapiens
RIAQDLGLELAELVPRLFRMASKDR

SEQ ID NO: 178 - protein, 25 aa, Homo sapiens
PTSNQQVKPLGLVLRKLLDREETPE

SEQ ID NO: 179 - protein, 25 aa, Homo sapiens
RIAQDLGLELAELVQRLFRVASKRH

SEQ ID NO: 180 - protein, 25 aa, Homo sapiens
ALLATQAGSAGGAVNKLVPRSVGAG

SEQ ID NO: 181 - protein, 25 aa, Homo sapiens
RIAQDLGLELAELVQRLFRVASKTH

SEQ ID NO: 182 - protein, 25 aa, Homo sapiens
VIIGPRGPGSQRLLLSLLLLAAWEV

SEQ ID NO: 183 - protein, 25 aa, Homo sapiens
ADRGSPALSSEALVRVLVLDANDNS

SEQ ID NO: 184 - protein, 25 aa, Homo sapiens
HYSVAEETESGSFVANLLKDLGLEI

SEQ ID NO: 185 - protein, 25 aa, Homo sapiens
TDRGSPALSSEALVRVLVLDANDNS

SEQ ID NO: 186 - protein, 25 aa, Homo sapiens
SDRGSPALSSEALVRVLVLDANDNS

SEQ ID NO: 187 - protein, 25 aa, Homo sapiens
TDHGSPALSSEALVRVLVLDANDNS

SEQ ID NO: 188 - protein, 25 aa, Homo sapiens
SDHGSPALSSEALVRVVVLDANDNS

SEQ ID NO: 189 - protein, 25 aa, Homo sapiens
TDRGFPALSSEALVRVLVLDANDNS

SEQ ID NO: 190 - protein, 25 aa, Homo sapiens
PPQRHPQRSEQVLLLTLLGTLWGAA

SEQ ID NO: 191 - protein, 25 aa, Homo sapiens
TPRFLKEELEVKILENAAPSSRFPL

SEQ ID NO: 192 - protein, 25 aa, Homo sapiens
RGPAGQRRMLFLFLLSLLDQVLSEP

SEQ ID NO: 193 - protein, 25 aa, Homo sapiens
SGGGSDEGLASAAARGLVEKVRQLL

SEQ ID NO: 194 - protein, 25 aa, Homo sapiens
TRMAAESRRVLLLAGRLAAQSLDTS

SEQ ID NO: 195 - protein, 25 aa, Homo sapiens
MGSEDHLGVIPRAIHDIFQKIKKFP

SEQ ID NO: 196 - protein, 25 aa, Homo sapiens
KRGPLLTALSAEAVASALHKLHQDL

SEQ ID NO: 197 - protein, 25 aa, Homo sapiens
EYHYVQEKASKLAAASLLLALYMKK

SEQ ID NO: 198 - protein, 25 aa, Homo sapiens
YIDENQDRYIKKLAKWVAIQSVSAW

SEQ ID NO: 199 - protein, 25 aa, Homo sapiens
ANPVEIRRGVMLAVDAVIAELKKQS

SEQ ID NO: 200 - protein, 25 aa, Homo sapiens
PVAVRLQMSERNILSRLANRAPEPT

SEQ ID NO: 201 - protein, 25 aa, Homo sapiens
ARERERLAHSRRAAARAVAAATAAV

SEQ ID NO: 202 - protein, 25 aa, Homo sapiens
LKTISVIPGLKTIVGALIQSVKKLS

SEQ ID NO: 203 - protein, 25 aa, Homo sapiens
LSRFEGMRVVVNALLGAIPSIMNVL

SEQ ID NO: 204 - protein, 25 aa, Homo sapiens
LYILTPFNPLRKIAIKILVHSLFSM

SEQ ID NO: 205 - protein, 25 aa, Homo sapiens
LYILTPFNPIRKLAIKILVHSLFNM

SEQ ID NO: 206 - protein, 25 aa, Homo sapiens
LSRFEGMRVVVNALVGAIPSIMNVL

SEQ ID NO: 207 - protein, 25 aa, Homo sapiens
LYILTPLNPVRKIAIKILVHSLFSM

SEQ ID NO: 208 - protein, 25 aa, Homo sapiens
LKTITVIPGLKTIVGALIQSVKKLS

SEQ ID NO: 209 - protein, 25 aa, Homo sapiens
LKTISVISGLKTIVGALIQSVKKLA

SEQ ID NO: 210 - protein, 25 aa, Homo sapiens
LYVLSPFHPVRRAAVKILVHSLFNM

SEQ ID NO: 211 - protein, 25 aa, Homo sapiens
LYILSPFNLIRRIAIKILIHSVFSM

SEQ ID NO: 212 - protein, 25 aa, Homo sapiens
EEQLRLRERELTALKGALKEEVASR

SEQ ID NO: 213 - protein, 25 aa, Homo sapiens
NYGKFEKGYLIFVVRFLFGLVNQER

SEQ ID NO: 214 - protein, 25 aa, Homo sapiens
RGALLAGALAAYAAYLVLGALLVAR

SEQ ID NO: 215 - protein, 25 aa, Homo sapiens
AQKVTRQEREEALVRGVFMKVVKPK

SEQ ID NO: 216 - protein, 25 aa, Homo sapiens
MLRYDVYGGENEVIPEVLRKSHSHF

SEQ ID NO: 217 - protein, 25 aa, Homo sapiens
ERQSAEVQGSLALVSRALEAAERAL

SEQ ID NO: 218 - protein, 25 aa, Homo sapiens
RSGYIEANELKGFLSDLLKKANRPY

SEQ ID NO: 219 - protein, 25 aa, Homo sapiens
VRRKLGEDWIFLVLLGLLMALVSWS

SEQ ID NO: 220 - protein, 25 aa, Homo sapiens
AYIVNYFMYVLWALLFAFLAVSLVK

SEQ ID NO: 221 - protein, 25 aa, Homo sapiens
LIHSVSDAFSGWLLMLLIGLLSGSL

SEQ ID NO: 222 - protein, 25 aa, Homo sapiens
YFPLKTLWRSFFAALVAAFTLRSIN

SEQ ID NO: 223 - protein, 25 aa, Homo sapiens
RKKGRESYIETELIFALAKTSRVSE

SEQ ID NO: 224 - protein, 25 aa, Homo sapiens
FAGSWRSGLAFLAVIKAIDPSLVDM

SEQ ID NO: 225 - protein, 25 aa, Homo sapiens
GYFGSDVKVAYQLATRLLAHESTQR

SEQ ID NO: 226 - protein, 25 aa, Homo sapiens
SPERFLSPLLGLFIQAVAATLATPP

SEQ ID NO: 227 - protein, 25 aa, Homo sapiens
LQEQLYVRRAALAARSLLDVLPFDD

SEQ ID NO: 228 - protein, 25 aa, Homo sapiens
WQERFLSPLLGRFLEGVAAVLATPA

SEQ ID NO: 229 - protein, 25 aa, Homo sapiens
YFSQDVRVTARLLAHLLAFESHQQG

SEQ ID NO: 230 - protein, 25 aa, Homo sapiens
LGSVIDISGLQRAVKEALSAVLPRV

SEQ ID NO: 231 - protein, 25 aa, Homo sapiens
SFMEHIAMPIYKLLQDLFPKAAELY

SEQ ID NO: 232 - protein, 25 aa, Homo sapiens
WVSFTSLGSLPSALRPLLSGLVGGA

SEQ ID NO: 233 - protein, 25 aa, Homo sapiens
HVIYQRVDKAVGLAEAALGLARANN

SEQ ID NO: 234 - protein, 25 aa, Homo sapiens
TDMKDMRLEAEAVVNDVLFAVNNMF

SEQ ID NO: 235 - protein, 25 aa, Homo sapiens
PKIVGRTKDVKEAVRKLAYQVLAEK

SEQ ID NO: 236 - protein, 25 aa, Homo sapiens
YGPTNFAPIINHVARFAAQAAHQGT

SEQ ID NO: 237 - protein, 25 aa, Homo sapiens
LAHLRARLKELAALEAAAKHEELVE

SEQ ID NO: 238 - protein, 25 aa, Homo sapiens
EEGGTPEQGVHRALQRLAHLLQADR

SEQ ID NO: 239 - protein, 25 aa, Homo sapiens
WKGGSASTWLTAFALRVLGQVNKYV

SEQ ID NO: 240 - protein, 25 aa, Homo sapiens
DVLPNFFYHSNQVVRMAALEVYVRR

SEQ ID NO: 241 - protein, 25 aa, Homo sapiens
RQVQAEVPGSPIFVMRLAKQSRHLE

SEQ ID NO: 242 - protein, 25 aa, Homo sapiens
SSQFHMATNSSMFLKQAFEGEYPKL

SEQ ID NO: 243 - protein, 25 aa, Homo sapiens
VRRLERKYSSIPVIQGIVNEVRQSM

SEQ ID NO: 244 - protein, 25 aa, Homo sapiens
TDPDLPPGYVQSLIRRVVNNVNIVI

SEQ ID NO: 245 - protein, 25 aa, Homo sapiens
GDVKSKTEALKKVIIMILNGEKLPG

SEQ ID NO: 246 - protein, 25 aa, Homo sapiens
TFTLSTIKTLEEAVGNIVKFLGMHP

SEQ ID NO: 247 - protein, 25 aa, Homo sapiens
LLFGAWAGVLGTALSLLIRAELGQP

SEQ ID NO: 248 - protein, 25 aa, Homo sapiens
RFIFNRVVLENEAVRAAAVSALAKF

SEQ ID NO: 249 - protein, 25 aa, Homo sapiens
RREEATRQGELPLVKEVLLVALGSR

SEQ ID NO: 250 - protein, 25 aa, Homo sapiens
NATLFTAAEIAPFVEILLTNLFKAL

SEQ ID NO: 251 - protein, 25 aa, Homo sapiens
SVNWKHKDAAIYLVTSLASKAQTQK

SEQ ID NO: 252 - protein, 25 aa, Homo sapiens
QRREGGGRNIGGIVGGIVNFISEAA

SEQ ID NO: 253 - protein, 25 aa, Homo sapiens
IEKESQRKSIDPALSMLIKSIKTKT

SEQ ID NO: 254 - protein, 25 aa, Homo sapiens
LDVIYWFRQIIAVVLGVIWGVLPLR

SEQ ID NO: 255 - protein, 25 aa, Homo sapiens
HRLLSTEWGLPSIVKSLIGAARTKT

SEQ ID NO: 256 - protein, 25 aa, Homo sapiens
NKSGNRSEKEVRAAALVLQTIWGYK

SEQ ID NO: 257 - protein, 25 aa, Homo sapiens
RHLTQQDPLSEAIVEKLIQSIQKVF

SEQ ID NO: 258 - protein, 25 aa, Homo sapiens
RFVKLAVMGLTVALGAAALAVVKSA

SEQ ID NO: 259 - protein, 25 aa, Homo sapiens
SVKRGNMVRAARALLSAVTRLLILA

SEQ ID NO: 260 - protein, 25 aa, Homo sapiens
SVKRGTMVRAARALLSAVTRLLILA

SEQ ID NO: 261 - protein, 25 aa, Homo sapiens
ARENAGPAIVISFLIAALASVLAGL

SEQ ID NO: 262 - protein, 25 aa, Homo sapiens
VSSSAPSVYSVQALSLLAEVLASLL

SEQ ID NO: 263 - protein, 25 aa, Homo sapiens
VYRSDEKEKAVPLISRLLYYVFPYL

SEQ ID NO: 264 - protein, 25 aa, Homo sapiens
RGPHGQLSPALPLASSVLMLLMSTL

SEQ ID NO: 265 - protein, 25 aa, Homo sapiens
TLRFLHASALLALASGLLAVLLAGL

SEQ ID NO: 266 - protein, 25 aa, Homo sapiens
FPNPRRRLRLQDLADRVVDASEDEH

SEQ ID NO: 267 - protein, 25 aa, Homo sapiens
LHYYSEREGLQDIVIGIIKTVAQQI

SEQ ID NO: 268 - protein, 25 aa, Homo sapiens
WKGAPTTSLISVAVTKIIAKVLEDN

SEQ ID NO: 269 - protein, 25 aa, Homo sapiens
YVASAFTLAVNIIAKKIVLKEQTGS

SEQ ID NO: 270 - protein, 25 aa, Homo sapiens
LLRILTDALVPYLVGQVVAGAQALQ

SEQ ID NO: 271 - protein, 25 aa, Homo sapiens
AEKTDEEEKEDRAAQSLLNKLIRSN

SEQ ID NO: 272 - protein, 25 aa, Homo sapiens
DEERRQLIQLRDILKSALQVEQKED

SEQ ID NO: 273 - protein, 25 aa, Homo sapiens
LGHPAAFGRATHAVVRALPESLGQH

SEQ ID NO: 274 - protein, 25 aa, Homo sapiens
ASFQRKWFEVAFVAEELVHSEIPAF

SEQ ID NO: 275 - protein, 25 aa, Homo sapiens
KVAFTGSTEVGKLIKEAAGKSNLKR

SEQ ID NO: 276 - protein, 25 aa, Homo sapiens
KIAFTGSTEVGKLIQEAAGRSNLKR

SEQ ID NO: 277 - protein, 25 aa, Homo sapiens
EAIKFINRQEKPLALYAFSNSRQVV

SEQ ID NO: 278 - protein, 25 aa, Homo sapiens
PRALLAALWALEAAGTAALRIGAFN

SEQ ID NO: 279 - protein, 25 aa, Homo sapiens
RLVSSPPSGVPGLALLALLALLALR

SEQ ID NO: 280 - protein, 25 aa, Homo sapiens
RVVLKGDVSLKDIIDPAFRASWIAQ

SEQ ID NO: 281 - protein, 25 aa, Homo sapiens
FQLPSRQPALSSFLGHLAAQVQAAL

SEQ ID NO: 282 - protein, 25 aa, Homo sapiens
RSLTSEREGLEGLLSKLLVLSSRNV

SEQ ID NO: 283 - protein, 25 aa, Homo sapiens
LIGPKVRITLMKFLPSVFMDAMRDN

SEQ ID NO: 284 - protein, 25 aa, Homo sapiens
QRPRAPRSALWLLAPPLLRWAPPLL

SEQ ID NO: 285 - protein, 25 aa, Homo sapiens
RQRLLGRSWSVPVIRHLFAPLKEYF

SEQ ID NO: 286 - protein, 25 aa, Homo sapiens
KRKLEDLSSEWKAVNRLLQELRAKQ

SEQ ID NO: 287 - protein, 25 aa, Homo sapiens
LPARVPRPGLSEALSLLLFAVVLSR

SEQ ID NO: 288 - protein, 25 aa, Homo sapiens
QRELQEALGARAALEALLGRLQAER

SEQ ID NO: 289 - protein, 25 aa, Homo sapiens
SARLRMVETLSNLLRSVVALSPPDL

SEQ ID NO: 290 - protein, 25 aa, Homo sapiens
ELAEHLNASLAFFLSDLLSLVDRGF

SEQ ID NO: 291 - protein, 25 aa, Homo sapiens
GPFRQQHFLAGLLLTELALALEPEA

SEQ ID NO: 292 - protein, 25 aa, Homo sapiens
LRAHGTHPAISTLARSAIFSVTYPS

SEQ ID NO: 293 - protein, 25 aa, Homo sapiens
IYEPPRYMSVNQAAQQLLEIVQNQR

SEQ ID NO: 294 - protein, 25 aa, Homo sapiens
HIYLYHHAQAHKALFGIFIPSQRRA

SEQ ID NO: 295 - protein, 25 aa, Homo sapiens
FNWRQAHMQARFVILRVLLEAGEGL

SEQ ID NO: 296 - protein, 25 aa, Homo sapiens
GVILGKWAILAILLGIALLFSVLLT

SEQ ID NO: 297 - protein, 25 aa, Homo sapiens
PNTELNVSRLEAVLSTIFYQLNKRM

SEQ ID NO: 298 - protein, 25 aa, Homo sapiens
RLDEEHRLIARYAARLAAESSSSQP

SEQ ID NO: 299 - protein, 25 aa, Homo sapiens
LTSVLGILASSTVLFMLFRPLFRWQ

SEQ ID NO: 300 - protein, 25 aa, Homo sapiens
PQELYESSHIESAINVAIPGIMLRR

SEQ ID NO: 301 - protein, 25 aa, Homo sapiens
SRELYESARIGGALSVALPALLLRR

SEQ ID NO: 302 - protein, 25 aa, Homo sapiens
DYYKKQVAQLKTLITMLIGQLSKGD

SEQ ID NO: 303 - protein, 25 aa, Homo sapiens
RDFVEEKLGSKYVVGRALDFATSFE

SEQ ID NO: 304 - protein, 25 aa, Homo sapiens
IGVKFLINEATTLADLLALRLHRVE

SEQ ID NO: 305 - protein, 25 aa, Homo sapiens
EGKKKQTNYLRTLINELVKGILPRS

SEQ ID NO: 306 - protein, 25 aa, Homo sapiens
GPSGSGKSMAWRVLLKALERLEGVE

SEQ ID NO: 307 - protein, 25 aa, Homo sapiens
KLVAEDIPLLFSLLSDVFPGVQYHR

SEQ ID NO: 308 - protein, 25 aa, Homo sapiens
LLSATELDKIRQALVAIFTHLRKIR

SEQ ID NO: 309 - protein, 25 aa, Homo sapiens
WRRFRWAIILFIILFILLLFLAIFI

SEQ ID NO: 310 - protein, 25 aa, Homo sapiens
PVRREVTDKEQEFAARAAKQLEYQQ

SEQ ID NO: 311 - protein, 25 aa, Homo sapiens
FPGDILMRMLKMLILPLIISSLITG

SEQ ID NO: 312 - protein, 25 aa, Homo sapiens
KLMVDFFNILNEIVMKLVIMIMWYS

SEQ ID NO: 313 - protein, 25 aa, Homo sapiens
FPGEILMRMLKLIILPLIISSMITG

SEQ ID NO: 314 - protein, 25 aa, Homo sapiens
PFMSAVSGRAYPAAITILETAQKIA

SEQ ID NO: 315 - protein, 25 aa, Homo sapiens
RNTFAERLSAVEAIANAISVVSSNG

SEQ ID NO: 316 - protein, 25 aa, Homo sapiens
NAAQTPRIPSRLLAILLFLLAMLLT

SEQ ID NO: 317 - protein, 25 aa, Homo sapiens
GLKELPMRNLQEILHGAVRFSNNPA

SEQ ID NO: 318 - protein, 25 aa, Homo sapiens
MVETQQLMRVYGALMWALGKVVGTP

SEQ ID NO: 319 - protein, 25 aa, Homo sapiens
HGIVSWDTFSVAFIKKIASFVNKSA

SEQ ID NO: 320 - protein, 25 aa, Homo sapiens
HGIVSWDMVSITFIKQIAGYVSQPM

SEQ ID NO: 321 - protein, 25 aa, Homo sapiens
VMNQQLQTKAMALLTALLQGASPVE

SEQ ID NO: 322 - protein, 25 aa, Homo sapiens
ESRVQQQEDEITVLKAALADVLRRL

SEQ ID NO: 323 - protein, 25 aa, Homo sapiens
GQFGVGFYSAFLVADKVIVTSKHNN

SEQ ID NO: 324 - protein, 25 aa, Homo sapiens
ELSSQLPERLSLVIGSILGALAFLL

SEQ ID NO: 325 - protein, 25 aa, Homo sapiens
MYRTHTRRALQTVAQLILELIEKQE

SEQ ID NO: 326 - protein, 25 aa, Homo sapiens
SGRAAALRQVVSAVTALVEAAERQP

SEQ ID NO: 327 - protein, 25 aa, Homo sapiens
RLQALRLEREEYVLLKALALANSDS

SEQ ID NO: 328 - protein, 25 aa, Homo sapiens
GESAGGESVSVLVLSPLAKNLFHRA

SEQ ID NO: 329 - protein, 25 aa, Homo sapiens
TPQKNNYNSIAAILIGVLLTSMLVA

SEQ ID NO: 330 - protein, 25 aa, Homo sapiens
ALRPAPALLAPAVLLGAALGLGLGL

SEQ ID NO: 331 - protein, 25 aa, Homo sapiens
NILVTTTQLIPALAKVLLYGLGIVF

SEQ ID NO: 332 - protein, 25 aa, Homo sapiens
NVLVTTTQLIPALAKVLLYGLGSVF

SEQ ID NO: 333 - protein, 25 aa, Homo sapiens
PADEKLQEKAWGAVVPLVGKLKKFY

SEQ ID NO: 334 - protein, 25 aa, Homo sapiens
VEQRKKLSSLLEFAQYLLAHSMFSR

SEQ ID NO: 335 - protein, 25 aa, Homo sapiens
NRLGIESPRSEKLARELLKELRTQV

SEQ ID NO: 336 - protein, 25 aa, Homo sapiens
QQRAQTMVQVKAVLGHLLAMSRSSS

SEQ ID NO: 337 - protein, 25 aa, Homo sapiens
SGQSKLNSWIQGVLSHILSALRFDK

SEQ ID NO: 338 - protein, 25 aa, Homo sapiens
ETRRGAYLNALKIAKLLLTAIGYGH

SEQ ID NO: 339 - protein, 25 aa, Homo sapiens
SQAYDNLSLSDHLLRAVLNLLRREV

SEQ ID NO: 340 - protein, 25 aa, Homo sapiens
WVVPVLPKGELEVLLEAAIDLSKKG

SEQ ID NO: 341 - protein, 25 aa, Homo sapiens
WVVPVLPKGELEVLLEAAIDLSVKG

SEQ ID NO: 342 - protein, 25 aa, Homo sapiens
RSNDKVYENVTGLVKAVIEMSSKIQ

SEQ ID NO: 343 - protein, 25 aa, Homo sapiens
ARTSSAEYNVNNLVSPVLFQEALWH

SEQ ID NO: 344 - protein, 25 aa, Homo sapiens
FRYMAQGKHIGKVVVQVLAEEPAVL

SEQ ID NO: 345 - protein, 25 aa, Homo sapiens
AVTIHPVTGSISVLNPAFLGLSRKL

SEQ ID NO: 346 - protein, 25 aa, Homo sapiens
PGPAPLRLLEWRVAAGAAVRIGSVL

SEQ ID NO: 347 - protein, 25 aa, Homo sapiens
MEEWDRYPRIGDILQKLAPFLKMYG

SEQ ID NO: 348 - protein, 25 aa, Homo sapiens
SGTKKSDFHSQMAVHKLAKSIPLRR

SEQ ID NO: 349 - protein, 25 aa, Homo sapiens
PSHSLLRLPLLQLLLLVVQAVGRGL

SEQ ID NO: 350 - protein, 25 aa, Homo sapiens
DVFIWLGRKSPRLVRAAALKLGQEL

SEQ ID NO: 351 - protein, 25 aa, Homo sapiens
HRLLQQLVLSGNLIKEAVRRLQRAV

SEQ ID NO: 352 - protein, 25 aa, Homo sapiens
LPHGSGLGTSSILAGTALAALQRAA

SEQ ID NO: 353 - protein, 25 aa, Homo sapiens
PESTARMQGAGKALHELLLSAQRQG

SEQ ID NO: 354 - protein, 25 aa, Homo sapiens
PKVDKWSRFLFPLAFGLFNIVYWVY

SEQ ID NO: 355 - protein, 25 aa, Homo sapiens
RLFTNLKDTSSKVIQSVANYAKGDL

SEQ ID NO: 356 - protein, 25 aa, Homo sapiens
ESLREVQLEELEAARDLVSKEGFRR

SEQ ID NO: 357 - protein, 25 aa, Homo sapiens
RPFLPYFNVSQQFATFVLKGSFSEI

SEQ ID NO: 358 - protein, 25 aa, Homo sapiens
NHGMWQTISVEELARNLVIKVNRDA

SEQ ID NO: 359 - protein, 25 aa, Homo sapiens
AGEDPKVTRAKFFIRDLFLRISTAT

SEQ ID NO: 360 - protein, 25 aa, Homo sapiens
PSVFGSNPKAHIAAKTVFHLAHRHG

SEQ ID NO: 361 - protein, 25 aa, Homo sapiens
HGRLIFITVLFSIIIWVVWISMLLR

SEQ ID NO: 362 - protein, 25 aa, Homo sapiens
NVLKDKMEKLKRLLQVAARKSQVTL

SEQ ID NO: 363 - protein, 25 aa, Homo sapiens
TINKFDKNFSAHLLDLLARLSIYST

SEQ ID NO: 364 - protein, 25 aa, Homo sapiens
IINNDTTITLAIVVDKLAPRLSQLK

SEQ ID NO: 365 - protein, 25 aa, Homo sapiens
GLLTEKAAPVMNVIHSIFSLVLKFR

SEQ ID NO: 366 - protein, 25 aa, Homo sapiens
IAKQELIAHAREAASRVLSALSDRQ

SEQ ID NO: 367 - protein, 25 aa, Homo sapiens
SYESMSEPPIAHLLRPVLPRAFAFP

SEQ ID NO: 368 - protein, 25 aa, Homo sapiens
GSAVEIVGLSKSAVRWLLELSKKNI

SEQ ID NO: 369 - protein, 25 aa, Homo sapiens
LRLETAPNISKDVIRQLLPKAPPLR

SEQ ID NO: 370 - protein, 25 aa, Homo sapiens
VLGDIHTTLLSAVIPNAFRLVKRKP

SEQ ID NO: 371 - protein, 25 aa, Homo sapiens
KDHAGVMGESNRLLSALIRHSKSKD

SEQ ID NO: 372 - protein, 25 aa, Homo sapiens
FNQLTQSASEQGLAKAVASVARLVI

SEQ ID NO: 373 - protein, 25 aa, Homo sapiens
GRQKLARFNAREFATLIIDILSEAK

SEQ ID NO: 374 - protein, 25 aa, Homo sapiens
KPVYYALEGSVAIAGAVIRWLRDNL

SEQ ID NO: 375 - protein, 25 aa, Homo sapiens
VGILSRRLQEALAAKEAADAELGQL

SEQ ID NO: 376 - protein, 25 aa, Homo sapiens
FLRRYPIARVFVIIYMALLHLWVMI

SEQ ID NO: 377 - protein, 25 aa, Homo sapiens
VLGLFWLLFASVVLILLLSWVGHVK

SEQ ID NO: 378 - protein, 25 aa, Homo sapiens
QASWVRPGVLWDVALVAVAALGLAL

SEQ ID NO: 379 - protein, 25 aa, Homo sapiens
FRLVSRRDYASEAIKGAVVGIDLGT

SEQ ID NO: 380 - protein, 25 aa, Homo sapiens
EDFKAKKKELEEIVQPIISKLYGSA

SEQ ID NO: 381 - protein, 25 aa, Homo sapiens
GQPKRYKGFSIDVLDALAKALGFKY

SEQ ID NO: 382 - protein, 25 aa, Homo sapiens
FQGKKNMTLAGRLAGPLFQTLIVAW

SEQ ID NO: 383 - protein, 25 aa, Homo sapiens
QKGHKSQREELDAILFIFEKILQLL

SEQ ID NO: 384 - protein, 25 aa, Homo sapiens
VKTQMQHGLISIAARTVITHLVNHL

SEQ ID NO: 385 - protein, 25 aa, Homo sapiens
VPTWDTIRDEEDVLDELLQYLGVTS

SEQ ID NO: 386 - protein, 25 aa, Homo sapiens
HEHIERRRKLYLAALPLAFEALIPN

SEQ ID NO: 387 - protein, 25 aa, Homo sapiens
GVWSEKGQVEVFALRRLLQVVEEPQ

SEQ ID NO: 388 - protein, 25 aa, Homo sapiens
MRPEDRMFHIRAVILRALSLAFLLS

SEQ ID NO: 389 - protein, 25 aa, Homo sapiens
SHTRGPEQQVKAILSELLQRENRVL

SEQ ID NO: 390 - protein, 25 aa, Homo sapiens
AMSKSRNPRLQTAAQELLEDLRTLE

SEQ ID NO: 391 - protein, 25 aa, Homo sapiens
ALGHKRNSGVPAFLTPLLRNIIISL

SEQ ID NO: 392 - protein, 25 aa, Homo sapiens
ENEDKWKRLSRQIADIILPMLAKQQ

SEQ ID NO: 393 - protein, 25 aa, Homo sapiens
FGDAALYQSLPTLARALAQYLVVVS

SEQ ID NO: 394 - protein, 25 aa, Homo sapiens
GLLKLQERVLNNVVIHLLGDEDPRV

SEQ ID NO: 395 - protein, 25 aa, Homo sapiens
NLSSRETSSLESFVRRVANIARTNA

SEQ ID NO: 396 - protein, 25 aa, Homo sapiens
GLPRPPMLLALLLATLLAAMLALLT

SEQ ID NO: 397 - protein, 25 aa, Homo sapiens
DQRKMLLVGSRKAAEQVIQDALNQL

SEQ ID NO: 398 - protein, 25 aa, Homo sapiens
GIALAYGSLLLMALLPIFFGALRSV

SEQ ID NO: 399 - protein, 25 aa, Homo sapiens
PNWKLKVSNLKMVLRSLVEYSQDVL

SEQ ID NO: 400 - protein, 25 aa, Homo sapiens
GDQLAQLNTVFQALPTAAWGATLRA

SEQ ID NO: 401 - protein, 25 aa, Homo sapiens
LLSSGRPKAVLQAVGQLVQKEQWDR

SEQ ID NO: 402 - protein, 25 aa, Homo sapiens
QKHAQQQKVVNKLIQFLISLVQSNR

SEQ ID NO: 403 - protein, 25 aa, Homo sapiens
AKHAQQQQVIRKIVQFIVTLVQNNQ

SEQ ID NO: 404 - protein, 25 aa, Homo sapiens
LNAARRYLGIEDLAGKVFVTSGLGG

SEQ ID NO: 405 - protein, 25 aa, Homo sapiens
FEKMISGMYLGEIVRHILLHLTSLG

SEQ ID NO: 406 - protein, 25 aa, Homo sapiens
IPANLSQNLEAAAATQVAVSVPKRR

SEQ ID NO: 407 - protein, 25 aa, Homo sapiens
IFHKNVFHYLMAFLRELLKNSAKNH

SEQ ID NO: 408 - protein, 25 aa, Homo sapiens
FEQWAHSEDLQSLLLRVANAVSVKG

SEQ ID NO: 409 - protein, 25 aa, Homo sapiens
KNSEATLPIAVRFAKTLLANSSPFN

SEQ ID NO: 410 - protein, 25 aa, Homo sapiens
SGKVSADNTVGRFLMSLVNQVPKIV

SEQ ID NO: 411 - protein, 25 aa, Homo sapiens
NPKIWNVHSVLNVLHSLVDKSNINR

SEQ ID NO: 412 - protein, 25 aa, Homo sapiens
LHGVMEYDLSLRFLENALAVSTKYH

SEQ ID NO: 413 - protein, 25 aa, Homo sapiens
ENGMYGKRKLLELIGHAVAHLKKAD

SEQ ID NO: 414 - protein, 25 aa, Homo sapiens
YRRKDEPDKAIELLKKALEYIPNNA

SEQ ID NO: 415 - protein, 25 aa, Homo sapiens
APSDPDAVSAEEALKYLLHLVDVNE

SEQ ID NO: 416 - protein, 25 aa, Homo sapiens
QLVKLLGASELPIVTPALRAIGNIV

SEQ ID NO: 417 - protein, 25 aa, Homo sapiens
SSNVENQLQATQAARKLLSREKQPP

SEQ ID NO: 418 - protein, 25 aa, Homo sapiens
PLLSHQEVKVQTAALRAVGNIVTGT

SEQ ID NO: 419 - protein, 25 aa, Homo sapiens
MRKEEPSNNVKLAATNALLNSLEFT

SEQ ID NO: 420 - protein, 25 aa, Homo sapiens
PPEHTSKFYAKGALQYLVPILTQTL

SEQ ID NO: 421 - protein, 25 aa, Homo sapiens
PYYDLFMPSLKHIVENAVQKELRLL

SEQ ID NO: 422 - protein, 25 aa, Homo sapiens
TAAEEARQMAAVLLRRLLSSAFDEV

SEQ ID NO: 423 - protein, 25 aa, Homo sapiens
LIAYRSRKRLETFLSLLVQNLAPAE

SEQ ID NO: 424 - protein, 25 aa, Homo sapiens
FVEPHKNMEVMGFLHGIFERLKQFL

SEQ ID NO: 425 - protein, 25 aa, Homo sapiens
LPVPQGPNPVVVVLQQVFQLIQKVL

SEQ ID NO: 426 - protein, 25 aa, Homo sapiens
NRERQKLMREQNILKQIFKLLQAPF

SEQ ID NO: 427 - protein, 25 aa, Homo sapiens
NRERQKLMREQNILAQVFGILKAPF

SEQ ID NO: 428 - protein, 25 aa, Homo sapiens
EKRVADPTLEKYVLSVVLDTINAFF

SEQ ID NO: 429 - protein, 25 aa, Homo sapiens
NRERQKLMREQNILKQVFGILKAPF

SEQ ID NO: 430 - protein, 25 aa, Homo sapiens
ETIQGLGAASAQFVSRLLPVLLSTA

SEQ ID NO: 431 - protein, 25 aa, Homo sapiens
HPAQEHFPKLLGLLFPLLARERHDR

SEQ ID NO: 432 - protein, 25 aa, Homo sapiens
LLRNPSSPRAKELAVSALGAIATAA

SEQ ID NO: 433 - protein, 25 aa, Homo sapiens
LMASPTRKPEPQVLAALLHALPLKE

SEQ ID NO: 434 - protein, 25 aa, Homo sapiens
SKALLKNRLLPPLLHTLFPIVAAEP

SEQ ID NO: 435 - protein, 25 aa, Homo sapiens
YMQAVNRERERQVVMAVLEALTGVL

SEQ ID NO: 436 - protein, 25 aa, Homo sapiens
DQYRQKEYVAPRVLQQAFNYLNQGV

SEQ ID NO: 437 - protein, 25 aa, Homo sapiens
ILADLNLSVSPFLLGRALWAASRFT

SEQ ID NO: 438 - protein, 25 aa, Homo sapiens
LGFENLVFSIFEFVHALLENSKFKS

SEQ ID NO: 439 - protein, 25 aa, Homo sapiens
PERWTNIPLLVKILKLIINELSNVM

SEQ ID NO: 440 - protein, 25 aa, Homo sapiens
RTSEFTAAFVGRLVSTLISKAGREL

SEQ ID NO: 441 - protein, 25 aa, Homo sapiens
LYNYASNQREEYLLLRLFKTALQEE

SEQ ID NO: 442 - protein, 25 aa, Homo sapiens
NRGARGQNALRQILAPVVKEIMDDK

SEQ ID NO: 443 - protein, 25 aa, Homo sapiens
YQDLLQLQYEGVAVMKLFDRAKVNV

SEQ ID NO: 444 - protein, 25 aa, Homo sapiens
LYNYASNQREEYLLLKLFKTALEEE

SEQ ID NO: 445 - protein, 25 aa, Homo sapiens
NRGARGQNTLRQLLAPVVKEIIDDK

SEQ ID NO: 446 - protein, 25 aa, Homo sapiens
WSPRKLPSSASTFLSPAFPGSQTHS

SEQ ID NO: 447 - protein, 25 aa, Homo sapiens
WLGGGVVPDAIVLAEEALDKAQEVL

SEQ ID NO: 448 - protein, 25 aa, Homo sapiens
GKPLERKLILVQVIPVVARMIYEMF

SEQ ID NO: 449 - protein, 25 aa, Homo sapiens
WRYMLLIFSLAFLASWLLFGIIFWV

SEQ ID NO: 450 - protein, 25 aa, Homo sapiens
WRWMMLVFSASFVVHWLVFAVLWYV

SEQ ID NO: 451 - protein, 25 aa, Homo sapiens
ALVIFEMPHLRDVALPALGAVLRGA

SEQ ID NO: 452 - protein, 25 aa, Homo sapiens
VGWIIAISLLVGILIFLLLAVLLWK

SEQ ID NO: 453 - protein, 25 aa, Homo sapiens
SNSIYPWSEVQTFLRRLVGKLFIDP

SEQ ID NO: 454 - protein, 25 aa, Homo sapiens
PIWIIVGSTLGGLLLLALLVLALWK

SEQ ID NO: 455 - protein, 25 aa, Homo sapiens
QGFTYTATAIQNVVHRLFHASYGAR

SEQ ID NO: 456 - protein, 25 aa, Homo sapiens
ARPRPRPLWVTVLALGALAGVGVGG

SEQ ID NO: 457 - protein, 25 aa, Homo sapiens
LVVLLSVMGAILLIGLAALLIWKLL

SEQ ID NO: 458 - protein, 25 aa, Homo sapiens
MAGPRPSPWARLLLAALISVSLSGT

SEQ ID NO: 459 - protein, 25 aa, Homo sapiens
AARQRQEIAAARAADALLKAVAASS

SEQ ID NO: 460 - protein, 25 aa, Homo sapiens
LSTLRYADRAKRIVNHAVVNEDPNA

SEQ ID NO: 461 - protein, 25 aa, Homo sapiens
SNINKSLTTLGLVISSLADQAAGKG

SEQ ID NO: 462 - protein, 25 aa, Homo sapiens
LSTLRYADRAKHIVNNAVVNEDPNA

SEQ ID NO: 463 - protein, 25 aa, Homo sapiens
QENLSLIGVANVFLESLFYDVKLQY

SEQ ID NO: 464 - protein, 25 aa, Homo sapiens
STSFRGGMGSGGLATGIAGGLAGMG

SEQ ID NO: 465 - protein, 25 aa, Homo sapiens
GFDVNIVEEELGIISRAVKHLFKSI

SEQ ID NO: 466 - protein, 25 aa, Homo sapiens
HQSVVYRKQAAMILNELVTGAAGLE

SEQ ID NO: 467 - protein, 25 aa, Homo sapiens
SLVHRLTRDAPLAVLRAFKVLRTLG

SEQ ID NO: 468 - protein, 25 aa, Homo sapiens
YLSVKQPVKLQEAARSVFLHLMKVD

SEQ ID NO: 469 - protein, 25 aa, Homo sapiens
KNVMEFLAPLKPVAIRIVRNAHGNK

SEQ ID NO: 470 - protein, 25 aa, Homo sapiens
GMRRGNMGHLTRIANAVVQNLERGP

SEQ ID NO: 471 - protein, 25 aa, Homo sapiens
RQDVLHWLNEEKVIQRLVELIHPSQ

SEQ ID NO: 472 - protein, 25 aa, Homo sapiens
EELVSIPWKVLKVVAKVIRALLRIL

SEQ ID NO: 473 - protein, 25 aa, Homo sapiens
GFIATPFIKLFQLIYYLVVAILKNI

SEQ ID NO: 474 - protein, 25 aa, Homo sapiens
TEILQHPERARDALRVLLHLVEKSL

SEQ ID NO: 475 - protein, 25 aa, Homo sapiens
RQDVVNWLNEEKIVQRLIEQIHPSK

SEQ ID NO: 476 - protein, 25 aa, Homo sapiens
ALHLATEMEELGLVTHLVTKLRANV

SEQ ID NO: 477 - protein, 25 aa, Homo sapiens
NSQWRQDMSISLAALELLSGLAKVK

SEQ ID NO: 478 - protein, 25 aa, Homo sapiens
EKGFYTERDASRLIFQVLDAVKYLH

SEQ ID NO: 479 - protein, 25 aa, Homo sapiens
EKGYYSERDAADAVKQILEAVAYLH

SEQ ID NO: 480 - protein, 25 aa, Homo sapiens
LQEFNARRKLKAAVKAVVASSRLGS

SEQ ID NO: 481 - protein, 25 aa, Homo sapiens
YALKRSFKELGLLLMYLAVGIFVFS

SEQ ID NO: 482 - protein, 25 aa, Homo sapiens
LLRLASTPDLRRFARSALNLVDLVA

SEQ ID NO: 483 - protein, 25 aa, Homo sapiens
STYFDMNLFLDIILKTVLENSGKRR

SEQ ID NO: 484 - protein, 25 aa, Homo sapiens
RGGVVRQYWSSSFLVDLLAVAAPVV

SEQ ID NO: 485 - protein, 25 aa, Homo sapiens
SALESTEEKLHDAASKLLNTVEETT

SEQ ID NO: 486 - protein, 25 aa, Homo sapiens
QENEPTVGVIPRVIQLLFKEIDKKS

SEQ ID NO: 487 - protein, 25 aa, Homo sapiens
ELSNHQKKRATEILNLLLKDLGEIG

SEQ ID NO: 488 - protein, 25 aa, Homo sapiens
EATAFGLGKEDAVLKVAVKMLKSTA

SEQ ID NO: 489 - protein, 25 aa, Homo sapiens
KDKLKPGAAEDDLVLEVVIMIGTVS

SEQ ID NO: 490 - protein, 25 aa, Homo sapiens
MEGKLHDPQLMGIIPRIARDIFNHI

SEQ ID NO: 491 - protein, 25 aa, Homo sapiens
SSAIIDHIFASKAVVNAAIPAYHLR

SEQ ID NO: 492 - protein, 25 aa, Homo sapiens
YLEVGLSGLSSKAAKDVLGFLRVVR

SEQ ID NO: 493 - protein, 25 aa, Homo sapiens
LEKLGYMDLASRLVTRVFKLLQNQI

SEQ ID NO: 494 - protein, 25 aa, Homo sapiens
EAPAYHLILEGILILWIIRLLFSKT

SEQ ID NO: 495 - protein, 25 aa, Homo sapiens
KTPSGIKLTINKLLDMAAQIAEGMA

SEQ ID NO: 496 - protein, 25 aa, Homo sapiens
ARGYVQDPFAALLVPGAARRAPLIH

SEQ ID NO: 497 - protein, 25 aa, Homo sapiens
DSLYFRLKTAGRLARAAVWEVDFPD

SEQ ID NO: 498 - protein, 25 aa, Homo sapiens
PLPSLRFLEELRLAGNALTYIPKGA

SEQ ID NO: 499 - protein, 25 aa, Homo sapiens
FWKAFWNITEMEVLSSLANMASATV

SEQ ID NO: 500 - protein, 25 aa, Homo sapiens
VTITLDLRQVFQVAYVIIKAANAPR

SEQ ID NO: 501 - protein, 25 aa, Homo sapiens
ERTNTRAKSLGEFIKELARDAEAVN

SEQ ID NO: 502 - protein, 25 aa, Homo sapiens
ETQKEIAEDELVAAEALLKKVKKLF

SEQ ID NO: 503 - protein, 25 aa, Homo sapiens
QSQAHQQRGLFPAVLNLASNALITT

SEQ ID NO: 504 - protein, 25 aa, Homo sapiens
NSARDAVRNLTEVVPQLLDQLRTVE

SEQ ID NO: 505 - protein, 25 aa, Homo sapiens
VKLSNLSNLSHDLVQEAIDHAQDLQ

SEQ ID NO: 506 - protein, 25 aa, Homo sapiens
GQPLPWELRLGLLLSVLAATLAQAP

SEQ ID NO: 507 - protein, 25 aa, Homo sapiens
ALDEKVRERLRMALERVAVLEEELE

SEQ ID NO: 508 - protein, 25 aa, Homo sapiens
DNTKSQLAMSANFLGSVLTLLQKQH

SEQ ID NO: 509 - protein, 25 aa, Homo sapiens
LLGGIKVKLLRGLLPNLVDNLVNRV

SEQ ID NO: 510 - protein, 25 aa, Homo sapiens
KGNKSSYHRLSELVEHVFPLLSKEQ

SEQ ID NO: 511 - protein, 25 aa, Homo sapiens
KNHKSTYERLGEVVELLFPPVARGP

SEQ ID NO: 512 - protein, 25 aa, Homo sapiens
TVLDQQQTPSRLAVTRVIQALAMKG

SEQ ID NO: 513 - protein, 25 aa, Homo sapiens
PGPLPSLPLEPSLLSGVVQALRGRL

SEQ ID NO: 514 - protein, 25 aa, Homo sapiens
SKTEQPAALALDLVNKLVYWVDLYL

SEQ ID NO: 515 - protein, 25 aa, Homo sapiens
AAKYRDHVTATQLIQKIINILTDKH

SEQ ID NO: 516 - protein, 25 aa, Homo sapiens
VSNMSITERLEHALEKAAPLLREIF

SEQ ID NO: 517 - protein, 25 aa, Homo sapiens
LSLLLLVTSVTLLVARVFQKAVDQS

SEQ ID NO: 518 - protein, 25 aa, Homo sapiens
DHLSQSKVIETQLAKPLFDALLRVA

SEQ ID NO: 519 - protein, 25 aa, Homo sapiens
SQAELVQKGSELVALRVALREARAT

SEQ ID NO: 520 - protein, 25 aa, Homo sapiens
SRHHHGSSIAGGLVKGALSVAASAY

SEQ ID NO: 521 - protein, 25 aa, Homo sapiens
GMAAGLYSELFTLLVSLVNRALKSS

SEQ ID NO: 522 - protein, 25 aa, Homo sapiens
AVVFSYIATLLYVVHAVFSLIRWKS

SEQ ID NO: 523 - protein, 25 aa, Homo sapiens
PPLNTIRDVSLKIAEKIVKDAYQEK

SEQ ID NO: 524 - protein, 25 aa, Homo sapiens
TFNDDIQGTASVAVAGLLAALRITK

SEQ ID NO: 525 - protein, 25 aa, Homo sapiens
KRKTTAAGGESALAPSVFKQAKDKV

SEQ ID NO: 526 - protein, 25 aa, Homo sapiens
KGKIKVIKKEGKAAEAVAAAVGTGA

SEQ ID NO: 527 - protein, 25 aa, Homo sapiens
FHPAVRNSSEVKFAVQAFAALNSNN

SEQ ID NO: 528 - protein, 25 aa, Homo sapiens
RRLGGLASQEPGAIIELFNSVLQFL

SEQ ID NO: 529 - protein, 25 aa, Homo sapiens
PPAAARAGGSPTAVRSILTKERRPE

SEQ ID NO: 530 - protein, 25 aa, Homo sapiens
RFSVVDMAALGGVLGALLLLALLGL

SEQ ID NO: 531 - protein, 25 aa, Homo sapiens
RGTVARGAGAGVVVKDAAAPSQPLR

SEQ ID NO: 532 - protein, 25 aa, Homo sapiens
AAFMKKYIHVAKIIKPVLTQESATY

SEQ ID NO: 533 - protein, 25 aa, Homo sapiens
DLRRKNEKRANRLLNNAFEELVAFQ

SEQ ID NO: 534 - protein, 25 aa, Homo sapiens
PFPSWRFPGLLLAAMVLLLYSFSDA

SEQ ID NO: 535 - protein, 25 aa, Homo sapiens
DVSTLHVQKIISAISELLERLKSYG

SEQ ID NO: 536 - protein, 25 aa, Homo sapiens
LATHRSTAKLLSVLAQVFTELAQKG

SEQ ID NO: 537 - protein, 25 aa, Homo sapiens
SLRNFYSHSLSGAVSNVFKILQPNT

SEQ ID NO: 538 - protein, 25 aa, Homo sapiens
VTSIAKAPAVQDLLTRLLQALHIDG

SEQ ID NO: 539 - protein, 25 aa, Homo sapiens
QQLQKQLKEAEQILATAVYQAKEKL

SEQ ID NO: 540 - protein, 25 aa, Homo sapiens
RLRHWWAIALTTAVTSAFLLAKVIL

SEQ ID NO: 541 - protein, 25 aa, Homo sapiens
MNMAKTSQTVATFLDELAQKLKPLG

SEQ ID NO: 542 - protein, 25 aa, Homo sapiens
KDYTKVDRSAAYAARWVAKSLVKGG

SEQ ID NO: 543 - protein, 25 aa, Homo sapiens
KDYTKVDRSAAYAARWVAKSLVKAG

SEQ ID NO: 544 - protein, 25 aa, Homo sapiens
VSPLKHFVLAKKAITAIFDQLLEFV

SEQ ID NO: 545 - protein, 25 aa, Homo sapiens
GYFPMIFRKAREFIEILFGISLTEV

SEQ ID NO: 546 - protein, 25 aa, Homo sapiens
RKPRFMSAWAQVIIASILISVQLTL

SEQ ID NO: 547 - protein, 25 aa, Homo sapiens
LKTAADRQAEDQVLRKLVDLVNQRD

SEQ ID NO: 548 - protein, 25 aa, Homo sapiens
YLMVMIGMFSFIIVAILVSTVKSKR

SEQ ID NO: 549 - protein, 25 aa, Homo sapiens
SSQPLTLEHVRYFLYQLLRGLKYMH

SEQ ID NO: 550 - protein, 25 aa, Homo sapiens
GARLALDEHVQFLVYQLLRGLKYIH

SEQ ID NO: 551 - protein, 25 aa, Homo sapiens
VKKPAGPSISKPAAKPAAAGAPPAK

SEQ ID NO: 552 - protein, 25 aa, Homo sapiens
RGERLHMFRVGGLVFHAIGQLLPHQ

SEQ ID NO: 553 - protein, 25 aa, Homo sapiens
WYVLVIISDLMTIIGSILKMEIKAK

SEQ ID NO: 554 - protein, 25 aa, Homo sapiens
SGKPRRKSNLPIFLPRVAGKLGKRP

SEQ ID NO: 555 - protein, 25 aa, Homo sapiens
KQKHFNEREASRVVRDVAAALDFLH

SEQ ID NO: 556 - protein, 25 aa, Homo sapiens
KHLERRDAESLKLLKEAIWEEKQGT

SEQ ID NO: 557 - protein, 25 aa, Homo sapiens
VYRKMPEHVLGRIAVAVVKGLTYLW

SEQ ID NO: 558 - protein, 25 aa, Homo sapiens
DLIFLRGIMESPIVRSLAKVIMVLW

SEQ ID NO: 559 - protein, 25 aa, Homo sapiens
STATNPQNGLSQILRLVLQELSLFY

SEQ ID NO: 560 - protein, 25 aa, Homo sapiens
ARRRLWGFSESLLIRGAAGRSLYFG

SEQ ID NO: 561 - protein, 25 aa, Homo sapiens
YYAKAFSKDLPRAVEILADIIQNST

SEQ ID NO: 562 - protein, 25 aa, Homo sapiens
HSVMDTLAVALRVAEEAIEEAISKA

SEQ ID NO: 563 - protein, 25 aa, Homo sapiens
AVSSAHRAASLEAVSYAIDTLKAKV

SEQ ID NO: 564 - protein, 25 aa, Homo sapiens
DEKKRRLMGLPSFLTEVARKELENL

SEQ ID NO: 565 - protein, 25 aa, Homo sapiens
SPSLQEKLKSFKAALIALYLLVFAV

SEQ ID NO: 566 - protein, 25 aa, Homo sapiens
SVRPLEFTKVKTFVSRIIDTLDIGP

SEQ ID NO: 567 - protein, 25 aa, Homo sapiens
SVRPQNFELVKRFVNQIVDFLDVSP

SEQ ID NO: 568 - protein, 25 aa, Homo sapiens
EEPYRRRNQILSVLAGLAAMVGYAL

SEQ ID NO: 569 - protein, 25 aa, Homo sapiens
VQLRRGLVGSRPVVTRVVIKAQGLV

SEQ ID NO: 570 - protein, 25 aa, Homo sapiens
LVDKGTEDKVVDVVRNLVFHLKKGY

SEQ ID NO: 571 - protein, 25 aa, Homo sapiens
KITINSHTTAGEVVEKLIRGLAMED

SEQ ID NO: 572 - protein, 25 aa, Homo sapiens
AGKQGLGPPSTPIAVHAAVKSKSLG

SEQ ID NO: 573 - protein, 25 aa, Homo sapiens
VSSTVGAAAVSALAGGALNGVFGRR

SEQ ID NO: 574 - protein, 25 aa, Homo sapiens
GKTVNTKRVIQYFATIAVTGEKKKE

SEQ ID NO: 575 - protein, 25 aa, Homo sapiens
VTKGQTVQQVYNAVGALAKAVYDKM

SEQ ID NO: 576 - protein, 25 aa, Homo sapiens
VTKGQTVEQVSNAVGALAKAVYEKM

SEQ ID NO: 577 - protein, 25 aa, Homo sapiens
VTKGQTVQQVYNAVGALAKAIYEKM

SEQ ID NO: 578 - protein, 25 aa, Homo sapiens
GKTVNTKRVIQYFATIAVTGEKKKD

SEQ ID NO: 579 - protein, 25 aa, Homo sapiens
VTKGQTVQQVYNAVGALAKAVYEKM

SEQ ID NO: 580 - protein, 25 aa, Homo sapiens
EQLKRNSQRAAEALQSVLDAEIRSR

SEQ ID NO: 581 - protein, 25 aa, Homo sapiens
QAAVQLALRAGQIIRKALTEEKRVS

SEQ ID NO: 582 - protein, 25 aa, Homo sapiens
IVWRRFKWVIIGLLFLLILLLFVAV

SEQ ID NO: 583 - protein, 25 aa, Homo sapiens
PLDDLDREDEVRLLKYLFTLIRAGM

SEQ ID NO: 584 - protein, 25 aa, Homo sapiens
IVLKNHHSRLSDLVNTAILIALNKR

SEQ ID NO: 585 - protein, 25 aa, Homo sapiens
HEAQLSEKISLQAIQQLVRKSYQAL

SEQ ID NO: 586 - protein, 25 aa, Homo sapiens
QGMSRVASVSQNAIVSAAGNIARTI

SEQ ID NO: 587 - protein, 25 aa, Homo sapiens
YRRAAEKWEVAEVVLEVFYKLLRDY

SEQ ID NO: 588 - protein, 25 aa, Homo sapiens
LFTFQKHVFSPIFIIGAFVAIFLGR

SEQ ID NO: 589 - protein, 25 aa, Homo sapiens
TSGRRWREISASLLYQALPSSPDHE

SEQ ID NO: 590 - protein, 25 aa, Homo sapiens
AEKQPPFTLIRSLLRKVLLPESFLI

SEQ ID NO: 591 - protein, 25 aa, Homo sapiens
AENMSITAKLERALEKVAPLLREIF

SEQ ID NO: 592 - protein, 25 aa, Homo sapiens
STVKIQNPMILKVVATLLKNSTPSA

SEQ ID NO: 593 - protein, 25 aa, Homo sapiens
KGGVGKSTFSAHLAHGLAEDENTQI

SEQ ID NO: 594 - protein, 25 aa, Homo sapiens
FIQEFPGSPAFAALTSIAQKILDAT

SEQ ID NO: 595 - protein, 25 aa, Homo sapiens
WRGPKKNALIKQFVSDVAWGELDYL

SEQ ID NO: 596 - protein, 25 aa, Homo sapiens
SARDLQNLMSWRFIMDLVSSLSRTY

SEQ ID NO: 597 - protein, 25 aa, Homo sapiens
SKLPKDQQDAKHILEHVFFQVVEFK

SEQ ID NO: 598 - protein, 25 aa, Homo sapiens
EPEPITASGSLKALRKLLTASVEVP

SEQ ID NO: 599 - protein, 25 aa, Homo sapiens
GRVLYREDTSPAVLGLAARYVRAGF

SEQ ID NO: 600 - protein, 25 aa, Homo sapiens
RTKRLHWSRLLFLLGMLIIGSTYQH

SEQ ID NO: 601 - protein, 25 aa, Homo sapiens
YKFGSRHSAESQILKHLLKNLFKIF

SEQ ID NO: 602 - protein, 25 aa, Homo sapiens
IVDNGVAPPARRLLRLVVRFAPQVE

SEQ ID NO: 603 - protein, 25 aa, Homo sapiens
GPVSARVIKALQVVDHAFGMLMEGL

SEQ ID NO: 604 - protein, 25 aa, Homo sapiens
SPESDAPGPVYAAASLAVSWVLRSV

SEQ ID NO: 605 - protein, 25 aa, Homo sapiens
SPPALPLASSFTALLQAAYESQALR

SEQ ID NO: 606 - protein, 25 aa, Homo sapiens
TMPHLSMQQVLLAAKQVLLYLRSTV

SEQ ID NO: 607 - protein, 25 aa, Homo sapiens
TQDMRLTFTLALFIAKAALQILKPE

SEQ ID NO: 608 - protein, 25 aa, Homo sapiens
VLQQRLGELERQLLRKVAELEDEKS

SEQ ID NO: 609 - protein, 25 aa, Homo sapiens
ISPLIQKSAANVVLFDIFVNILTHN

SEQ ID NO: 610 - protein, 25 aa, Homo sapiens
VQGNVTSTESMDFLKYVVDKLNFKP

SEQ ID NO: 611 - protein, 25 aa, Homo sapiens
GFKLLWILLLATLVGLLLQRLAARL

SEQ ID NO: 612 - protein, 25 aa, Homo sapiens
VYVRDLGHVALYVVAAVVSVAYLGF

SEQ ID NO: 613 - protein, 25 aa, Homo sapiens
RKPGNVLKTLEPILITIIAMSALGV

SEQ ID NO: 614 - protein, 25 aa, Homo sapiens
DYVPIGPRFSNLVLQALLVLLKKAP

SEQ ID NO: 615 - protein, 25 aa, Homo sapiens
YMIHHGDWFSGKAVGLLVLHLSGMV

SEQ ID NO: 616 - protein, 25 aa, Homo sapiens
ILWSGWASNSNYALIGALRAVAQTI

SEQ ID NO: 617 - protein, 25 aa, Homo sapiens
IRFLKGMGYSTHAAQQVLHAASGNL

SEQ ID NO: 618 - protein, 25 aa, Homo sapiens
KQIQRTKRGLEILAKRAAETVVDPE

SEQ ID NO: 619 - protein, 25 aa, Homo sapiens
ELLNMYVGESERAVRQVFQRAKNSA

SEQ ID NO: 620 - protein, 25 aa, Homo sapiens
RLSLVAGAYVAGLISALVRTVSAFT

SEQ ID NO: 621 - protein, 25 aa, Homo sapiens
WRRLPGAGLARGFLHPAATVEDAAQ

SEQ ID NO: 622 - protein, 25 aa, Homo sapiens
IVFNAHIKGVETIANDVVSLATKAR

SEQ ID NO: 623 - protein, 25 aa, Homo sapiens
DFLNNPFKQENVLARMVASRITNYP

SEQ ID NO: 624 - protein, 25 aa, Homo sapiens
NLRRDVYIRIASLLKTLLKTEEWVL

SEQ ID NO: 625 - protein, 25 aa, Homo sapiens
AAETLLSSLLGLLLLGLLLPASLTG

SEQ ID NO: 626 - protein, 25 aa, Homo sapiens
KRENPQLKQIEGLVKELLEREGLTA

SEQ ID NO: 627 - protein, 25 aa, Homo sapiens
KRVAYARVPSKDLLFSIVEEETGKD

SEQ ID NO: 628 - protein, 25 aa, Homo sapiens
TVPVFFNQAERRAVLQAARMAGLKV

SEQ ID NO: 629 - protein, 25 aa, Homo sapiens
VGGATRVPRVQEVLLKAVGKEELGK

SEQ ID NO: 630 - protein, 25 aa, Homo sapiens
DQKAYKEGKLQKALEDAFLAIDAKL

SEQ ID NO: 631 - protein, 25 aa, Homo sapiens
TKYKMGGDIANRVLRSLVEASSSGV

SEQ ID NO: 632 - protein, 25 aa, Homo sapiens
NRPSIPYAFSKFLLPIVVRYLADQN

SEQ ID NO: 633 - protein, 25 aa, Homo sapiens
HRSPQLLLELDNVISVLFQNSKERG

SEQ ID NO: 634 - protein, 25 aa, Homo sapiens
HRHMRTIREVRTLVTRVITDVYYVD

SEQ ID NO: 635 - protein, 25 aa, Homo sapiens
AEQFAPPDIAPPLLIKLVEAIEKKG

SEQ ID NO: 636 - protein, 25 aa, Homo sapiens
RQVQETLNLEPDVAQHLLAHSHWGA

SEQ ID NO: 637 - protein, 25 aa, Homo sapiens
VAMGEMEADVQALVRRAARQLAESG

SEQ ID NO: 638 - protein, 25 aa, Homo sapiens
VELLTNQVGEKMVVVQALRLLYLLM

SEQ ID NO: 639 - protein, 25 aa, Homo sapiens
HPPYLVSKELMSLVSGLLQPVPERR

SEQ ID NO: 640 - protein, 25 aa, Homo sapiens
STMSPLGSGAFGFVWTAVDKEKNKE

SEQ ID NO: 641 - protein, 25 aa, Homo sapiens
TSRRRNVTFSQQVANILLNGVKYES

SEQ ID NO: 642 - protein, 25 aa, Homo sapiens
LFTLDEQSGLLTVAWPLARRANSVV

SEQ ID NO: 643 - protein, 25 aa, Homo sapiens
KVTDHGKPTLSAVAKLIIRSVSGSL

SEQ ID NO: 644 - protein, 25 aa, Homo sapiens
SHINAATGTSASLVYRLVSKAGDAP

SEQ ID NO: 645 - protein, 25 aa, Homo sapiens
NGETLVFEESNWFIINVIKLVWRYG

SEQ ID NO: 646 - protein, 25 aa, Homo sapiens
KADVNLSHSERGALQDALRRLLGLF

SEQ ID NO: 647 - protein, 25 aa, Homo sapiens
NQRKAAHSAELEAVLLALARIRRAL

SEQ ID NO: 648 - protein, 25 aa, Homo sapiens
ISVQLKKTSEVDLAKPLVKFIQQTY

SEQ ID NO: 649 - protein, 25 aa, Homo sapiens
NRSIAQMREATTLANGVLASLNLPA

SEQ ID NO: 650 - protein, 25 aa, Homo sapiens
PAKTMQGSEVVNVLKSLLSNLDEVK

SEQ ID NO: 651 - protein, 25 aa, Homo sapiens
VTEFNSQTSAKIFAARILNHLLLFV

SEQ ID NO: 652 - protein, 25 aa, Homo sapiens
RLALFPGVALLLAAARLAAASDVLE

SEQ ID NO: 653 - protein, 25 aa, Homo sapiens
YQGGRTGEAIVDAALSALRQLVKDR

SEQ ID NO: 654 - protein, 25 aa, Homo sapiens
FSEMLAASFSIAVVAYAIAVSVGKV

SEQ ID NO: 655 - protein, 25 aa, Homo sapiens
RHKIPVPIPIEVIVTIIATAISYGA

SEQ ID NO: 656 - protein, 25 aa, Homo sapiens
ELLSKYIGASEQAVRDIFIRAQAAK

SEQ ID NO: 657 - protein, 25 aa, Homo sapiens
GPGPPQLLVSRALLRLLALGSGAWV

SEQ ID NO: 658 - protein, 25 aa, Homo sapiens
EFFTHLDKRSLPALTNIIKILRHDI

SEQ ID NO: 659 - protein, 25 aa, Homo sapiens
FESIGKFGLALAVAGGVVNSALYNV

SEQ ID NO: 660 - protein, 25 aa, Homo sapiens
MDRSSKRRQVKPLAASLLEALDYDS

SEQ ID NO: 661 - protein, 25 aa, Homo sapiens
TVTWGNYGKSYSVALYLVRQLTSSE

SEQ ID NO: 662 - protein, 25 aa, Homo sapiens
TTAKRRLKQSVHLARRVLQLEKQNS

SEQ ID NO: 663 - protein, 25 aa, Homo sapiens
RETAEPFLFVDEFLTYLFSRENSIW

SEQ ID NO: 664 - protein, 25 aa, Homo sapiens
LVSTLVPLGLVLAVGAVAVGVARAR

SEQ ID NO: 665 - protein, 25 aa, Homo sapiens
EVARGKRAALFFAAVAIVLGLPLWW

SEQ ID NO: 666 - protein, 25 aa, Homo sapiens
FTSFDQVAQLSSAARGLAASLLFLL

SEQ ID NO: 667 - protein, 25 aa, Homo sapiens
GAWARWLLVALTAATALVRLAQLGA

SEQ ID NO: 668 - protein, 25 aa, Homo sapiens
LHAAVTLRLEFPAAGRALAALSVRP

SEQ ID NO: 669 - protein, 25 aa, Homo sapiens
RMVASQAYNLTSALMRILMRSRVLN

SEQ ID NO: 670 - protein, 25 aa, Homo sapiens
ESSTNREKYLKSVLRELVTYLLFLI

SEQ ID NO: 671 - protein, 25 aa, Homo sapiens
LLHSRNEGTATYAAAVLFRISEDKN

SEQ ID NO: 672 - protein, 25 aa, Homo sapiens
RQFRTGKVTVEKVIKILITIVEEVE

SEQ ID NO: 673 - protein, 25 aa, Homo sapiens
RQFRTGRITVEKIIKIIITVVEEQE

SEQ ID NO: 674 - protein, 25 aa, Homo sapiens
GPGPRFLLLLPLLLPPAASASDRPR

SEQ ID NO: 675 - protein, 25 aa, Homo sapiens
DILKPGGGTSGGLLGGLLGKVTSVI

SEQ ID NO: 676 - protein, 25 aa, Homo sapiens
VLRGLDITLVHDIVNMLIHGLQFVI

SEQ ID NO: 677 - protein, 25 aa, Homo sapiens
LGQVPLIVGILLVLMAVVLASLIYR

SEQ ID NO: 678 - protein, 25 aa, Homo sapiens
PDKRQILLQEEKLLLAVLKTSLIGM

SEQ ID NO: 679 - protein, 25 aa, Homo sapiens
GLNPSLMAPSQFAAGGALLSLNPGT

SEQ ID NO: 680 - protein, 25 aa, Homo sapiens
ITLGYTQADVGLILGVLFGKVFSQK

SEQ ID NO: 681 - protein, 25 aa, Homo sapiens
ITLGYTQADVGLILGVLFGKVFSQT

SEQ ID NO: 682 - protein, 25 aa, Homo sapiens
LFSKYTNSKIPYFLLFLIFLITVYH

SEQ ID NO: 683 - protein, 25 aa, Homo sapiens
DFVDRGFYSVETFLLLLALKVRYPD

SEQ ID NO: 684 - protein, 25 aa, Homo sapiens
LLLARAASLSLGFLFLLFFWLDRSV

SEQ ID NO: 685 - protein, 25 aa, Homo sapiens
LVQIIKKTESDAALHPLLQEIYRDM

SEQ ID NO: 686 - protein, 25 aa, Homo sapiens
QTWLSALRPSGPALSGLLSLEAEEN

SEQ ID NO: 687 - protein, 25 aa, Homo sapiens
SMEGVTFLQAKQIALHALSLVGEKQ

SEQ ID NO: 688 - protein, 25 aa, Homo sapiens
VVGTTTTTPSPSAIKAAAKSAALQV

SEQ ID NO: 689 - protein, 25 aa, Homo sapiens
NDLTRNRFFENPALWELLFHSIHDA

SEQ ID NO: 690 - protein, 25 aa, Homo sapiens
PPSGIHLSASRTLAPTLLYSSPPSH

SEQ ID NO: 691 - protein, 25 aa, Homo sapiens
KRLFNVDRHVGMAVAGLLADARSLA

SEQ ID NO: 692 - protein, 25 aa, Homo sapiens
RSNFGYNIPLKHLADRVAMYVHAYT

SEQ ID NO: 693 - protein, 25 aa, Homo sapiens
VLYEDEGFRSRQFAALVASKVFYHL

SEQ ID NO: 694 - protein, 25 aa, Homo sapiens
HLLRYGEPTLRRAVPLALALISVSN

SEQ ID NO: 695 - protein, 25 aa, Homo sapiens
GYGHMWSQNATNLVSSLLTLLKQLE

SEQ ID NO: 696 - protein, 25 aa, Homo sapiens
KSLLDPKVAARLAVAEALTNLVFAL

SEQ ID NO: 697 - protein, 25 aa, Homo sapiens
AQVGLGVGTSLLALGVIIIVLMYRR

SEQ ID NO: 698 - protein, 25 aa, Homo sapiens
TRLLSMKGTLQKFVDDLFQVILSTS

SEQ ID NO: 699 - protein, 25 aa, Homo sapiens
LVVPLPGRDLLLVARGLAGKLSAGV

SEQ ID NO: 700 - protein, 25 aa, Homo sapiens
GELSARQMHLARFLRMLLRLADEFG

SEQ ID NO: 701 - protein, 25 aa, Homo sapiens
IDLVSKLLYSRGLLIDLLIKSNVSR

SEQ ID NO: 702 - protein, 25 aa, Homo sapiens
IDLVSKLLYSQGLLIDLLIKSDVSR

SEQ ID NO: 703 - protein, 25 aa, Homo sapiens
AIFTGHSAVVEDVAWHLLHESLFGS

SEQ ID NO: 704 - protein, 25 aa, Homo sapiens
EHPAIRTLSARAAAAFVLANENNIA

SEQ ID NO: 705 - protein, 25 aa, Homo sapiens
MARGGRGRRLGLALGLLLALVLAPR

SEQ ID NO: 706 - protein, 25 aa, Homo sapiens
FEEYLRALDVNVALRKIANLLKPDK

SEQ ID NO: 707 - protein, 25 aa, Homo sapiens
MEDYLQALNISLAVRKIALLLKPDK

SEQ ID NO: 708 - protein, 25 aa, Homo sapiens
HSYVSVKAKVSSIAQEILKVVAEKI

SEQ ID NO: 709 - protein, 25 aa, Homo sapiens
RLLWRLPAPVLVVLRYLFTFLNHLA

SEQ ID NO: 710 - protein, 25 aa, Homo sapiens
PKPVVPKTNVKALVPNLLRAIEAGI

SEQ ID NO: 711 - protein, 25 aa, Homo sapiens
YPRKFNETQIKQALRGVLESVKHNL

SEQ ID NO: 712 - protein, 25 aa, Homo sapiens
KKNFESLSEAFSVASAAAVLSHNRY

SEQ ID NO: 713 - protein, 25 aa, Homo sapiens
VSTTVAKAMAREAAQRVAESSRLEK

SEQ ID NO: 714 - protein, 25 aa, Homo sapiens
DPVLRKKNGATPFILAAIAGSVKLL

SEQ ID NO: 715 - protein, 25 aa, Homo sapiens
AHKATNKSSETLALLEILKHIAITE

SEQ ID NO: 716 - protein, 25 aa, Homo sapiens
LNYVASQPKLAPFVIQALIQVIAKI

SEQ ID NO: 717 - protein, 25 aa, Homo sapiens
YGDNHFDNVLQAFVKMLLSVSHSDL

SEQ ID NO: 718 - protein, 25 aa, Homo sapiens
WVSVLLKKTEKAFLAHLASAVAELR

SEQ ID NO: 719 - protein, 25 aa, Homo sapiens
FMKLVGMPYLHEVLKPVISRVFEEK

SEQ ID NO: 720 - protein, 25 aa, Homo sapiens
QHADPQTSRSLLLLAKAVQSIGNLG

SEQ ID NO: 721 - protein, 25 aa, Homo sapiens
INLKRTWEKLLLAARAIVAIENPAD

SEQ ID NO: 722 - protein, 25 aa, Homo sapiens
SQGMVGQLAARRAAGVVLEMIREGK

SEQ ID NO: 723 - protein, 25 aa, Homo sapiens
EQGKRNFSKAMSVAKQVFNSLTEYI

SEQ ID NO: 724 - protein, 25 aa, Homo sapiens
IDEASWMKRLAVFAQPIVSRARPEL

SEQ ID NO: 725 - protein, 25 aa, Homo sapiens
NYLSRNFYTLRFLALFLAFAINFIL

SEQ ID NO: 726 - protein, 25 aa, Homo sapiens
QAGKGEALRIRAILRSLVPLEDLVG

SEQ ID NO: 727 - protein, 25 aa, Homo sapiens
RVRRLRRLTAREAATAVAALLWAAV

SEQ ID NO: 728 - protein, 25 aa, Homo sapiens
TAAAGATARVVAAAGRALRGLSYRS

SEQ ID NO: 729 - protein, 25 aa, Homo sapiens
VEKSPHEQEIKFFAKILLPLINQYF

SEQ ID NO: 730 - protein, 25 aa, Homo sapiens
ARNFYNMRMLALFVAFAINFILLFY

SEQ ID NO: 731 - protein, 25 aa, Homo sapiens
DTPSIEKRFAYSFLQQLIRYVDEAH

SEQ ID NO: 732 - protein, 25 aa, Homo sapiens
EQGQRNFSKAIQVAKQVFNTLTEYI

SEQ ID NO: 733 - protein, 25 aa, Homo sapiens
GEHFPYEQEIKFFAKVVLPLIDQYF

SEQ ID NO: 734 - protein, 25 aa, Homo sapiens
EESGMAWKEILNLLYKLLAALIRGN

SEQ ID NO: 735 - protein, 25 aa, Homo sapiens
HYLARNFYNLRFLALFVAFAINFIL

SEQ ID NO: 736 - protein, 25 aa, Homo sapiens
QTGKGEAIRIRSILRSLVPTEDLVG

SEQ ID NO: 737 - protein, 25 aa, Homo sapiens
TEKSPRDQEIKFFAKVLLPLVDQYF

SEQ ID NO: 738 - protein, 25 aa, Homo sapiens
TLYQQARLHERGAAEMVLQMISASK

SEQ ID NO: 739 - protein, 25 aa, Homo sapiens
FLSGQGLAGIFAALAMLLSMASGVD

SEQ ID NO: 740 - protein, 25 aa, Homo sapiens
SNSLAYYNMANGAVIHLALKERGGR

SEQ ID NO: 741 - protein, 25 aa, Homo sapiens
LQLLSGHPPASEAVASVLSFLYDKK

SEQ ID NO: 742 - protein, 25 aa, Homo sapiens
QISLEGYEKALEFATLAARLSTVTG

SEQ ID NO: 743 - protein, 25 aa, Homo sapiens
HTSTLAAMKLMTALVNVALNLSINM

SEQ ID NO: 744 - protein, 25 aa, Homo sapiens
APPVAAGVGAVLAAGALLGLVAGAL

SEQ ID NO: 745 - protein, 25 aa, Homo sapiens
DMVKSKWGLALAAVVTVLSSLLMSV

SEQ ID NO: 746 - protein, 25 aa, Homo sapiens
GMTWSHGLSVSKVLHKAFVEVTEEG

SEQ ID NO: 747 - protein, 25 aa, Homo sapiens
AGKSGGSAGEITFLEALARSESKRD

SEQ ID NO: 748 - protein, 25 aa, Homo sapiens
ILQKYIERIITRFAPMLVPYIWQNQ

SEQ ID NO: 749 - protein, 25 aa, Homo sapiens
DAQLDYHRQAVQILDELAEKLKRRM

SEQ ID NO: 750 - protein, 25 aa, Homo sapiens
FKKDPPLAAVTTAVQELLRLAQAHP

SEQ ID NO: 751 - protein, 25 aa, Homo sapiens
RGLGVHHSGILPILKEIVEMLFSRG

SEQ ID NO: 752 - protein, 25 aa, Homo sapiens
RRIDLSNNQIAEIAPDAFQGLRSLN

SEQ ID NO: 753 - protein, 25 aa, Homo sapiens
SSAGPVRPELWLLLWAAAWRLGASA

SEQ ID NO: 754 - protein, 25 aa, Homo sapiens
VTVLFALVLSGALIILVASPLRALR

SEQ ID NO: 755 - protein, 25 aa, Homo sapiens
PGGMNRKTQETAVAMHVAANSIQNR

SEQ ID NO: 756 - protein, 25 aa, Homo sapiens
TSTWLDDVEERLFVATALLPEETET

SEQ ID NO: 757 - protein, 25 aa, Homo sapiens
INSQLARHTSPSVISDLFTDIKKGH

SEQ ID NO: 758 - protein, 25 aa, Homo sapiens
QHVDQRRQGLEDFLRKVLQNALLLS

SEQ ID NO: 759 - protein, 25 aa, Homo sapiens
PDIPEWRKDIGNVIKRALVKVTSVP

SEQ ID NO: 760 - protein, 25 aa, Homo sapiens
LQHRHRLPDLQAILRRILNEEETSP

SEQ ID NO: 761 - protein, 25 aa, Homo sapiens
HNNRRLQAESESAATRLLLASKQLG

SEQ ID NO: 762 - protein, 25 aa, Homo sapiens
RAAPRGPGAELQAAGSLVWGVRAAP

SEQ ID NO: 763 - protein, 25 aa, Homo sapiens
RNVFFSPMSISSALAMVFMGAKGST

SEQ ID NO: 764 - protein, 25 aa, Homo sapiens
QLAKQKAQEAEKLLNNVISKLLPTN

SEQ ID NO: 765 - protein, 25 aa, Homo sapiens
SLLDKHSQIINKFVNSVINTLKSTV

SEQ ID NO: 766 - protein, 25 aa, Homo sapiens
IQSLTMYPRLGGFVMNILSRLIMKQ

SEQ ID NO: 767 - protein, 25 aa, Homo sapiens
VHPAISSINLTTALGSLANIARQRP

SEQ ID NO: 768 - protein, 25 aa, Homo sapiens
PFYDPEGGSITQVARVVIERIARKG

SEQ ID NO: 769 - protein, 25 aa, Homo sapiens
PLKLSRTPALLALALPLAAALAFSD

SEQ ID NO: 770 - protein, 25 aa, Homo sapiens
MVGPAPRRRLRPLAALALVLALAPG

SEQ ID NO: 771 - protein, 25 aa, Homo sapiens
KTGSFKIRGALNAVRSLVPDALERK

SEQ ID NO: 772 - protein, 25 aa, Homo sapiens
KHGPGRWVVLAAVLIGLLLVLLGIG

SEQ ID NO: 773 - protein, 25 aa, Homo sapiens
SNRGLTKENLVFLAQKLFNNSSSHL

SEQ ID NO: 774 - protein, 25 aa, Homo sapiens
DASKALLGRLTTLIELLLPKLEEWK

SEQ ID NO: 775 - protein, 25 aa, Homo sapiens
KGVDLRNAQVTELLQRLLHRAFVVE

SEQ ID NO: 776 - protein, 25 aa, Homo sapiens
PAAGLGPGHARHVLRSLVNQSVQDG

SEQ ID NO: 777 - protein, 25 aa, Homo sapiens
PDNTGRGYVLRRILRRAVRYAHEKL

SEQ ID NO: 778 - protein, 25 aa, Homo sapiens
RRPIMSNHTATHILNFALRSVLGEA

SEQ ID NO: 779 - protein, 25 aa, Homo sapiens
FLAGETESLADIVLWGALYPLLQDP

SEQ ID NO: 780 - protein, 25 aa, Homo sapiens
YHQLLEKVRIRDALRSILTISRHGN

SEQ ID NO: 781 - protein, 25 aa, Homo sapiens
FLKGVLVFLEQALIQYALRTLGSRG

SEQ ID NO: 782 - protein, 25 aa, Homo sapiens
WEVGVYAAGALALLGIAAVSLWKLW

SEQ ID NO: 783 - protein, 25 aa, Homo sapiens
WLYLQDQNKAADAVGEILLSLSYLP

SEQ ID NO: 784 - protein, 25 aa, Homo sapiens
TRPWLLDPKILKFVVFIVAVLLPVR

SEQ ID NO: 785 - protein, 25 aa, Homo sapiens
TWKDRFPGYLMNFASILFMIALTFS

SEQ ID NO: 786 - protein, 25 aa, Homo sapiens
EVVKLHPHELNNLLSKVLIYLRSAN

SEQ ID NO: 787 - protein, 25 aa, Homo sapiens
YALAVRQDVINTLLPKVLTRIIEGL

SEQ ID NO: 788 - protein, 25 aa, Homo sapiens
MADPDVLTEVPAALKRLAKYVIRGF

SEQ ID NO: 789 - protein, 25 aa, Homo sapiens
FSQGKMYGYVDTLLTMLAMLLKVAM

SEQ ID NO: 790 - protein, 25 aa, Homo sapiens
KLQEIMMHVIWAALAFAAIQLLGML

SEQ ID NO: 791 - protein, 25 aa, Homo sapiens
DTEFAKQTSLDAVAQAVVDRVKRIH

SEQ ID NO: 792 - protein, 25 aa, Homo sapiens
LEFAIMRIEALKLARQIALASRSHQ

SEQ ID NO: 793 - protein, 25 aa, Homo sapiens
RANTHIRDFLQVFIYRLFWKSKDRP

SEQ ID NO: 794 - protein, 25 aa, Homo sapiens
DIERPTYTNLNRLISQIVSSITASL

SEQ ID NO: 795 - protein, 25 aa, Homo sapiens
QTIIAGWREATKAAREALLSSAVDH

SEQ ID NO: 796 - protein, 25 aa, Homo sapiens
LDTYLGKYWAIKLATNAAVTVLRVD

SEQ ID NO: 797 - protein, 25 aa, Homo sapiens
NFGAFSINPAMMAAAQAALQSSWGM

SEQ ID NO: 798 - protein, 25 aa, Homo sapiens
QKLYIPRSTATAALGAAARLATSRS

SEQ ID NO: 799 - protein, 25 aa, Homo sapiens
EEEEKVSQPEVGAAIKIIRQLMEKF

SEQ ID NO: 800 - protein, 25 aa, Homo sapiens
LSKKLIYFQLHRALKMIVDPVEPHG

SEQ ID NO: 801 - protein, 25 aa, Homo sapiens
GSRGTTAGSSGDALGKALASIYSPD

SEQ ID NO: 802 - protein, 25 aa, Homo sapiens
LPSIPAQPISADIASRLLRKLKGPV

SEQ ID NO: 803 - protein, 25 aa, Homo sapiens
NLHKVLQGRLPAVAQAVAQLAGQLL

SEQ ID NO: 804 - protein, 25 aa, Homo sapiens
QRDAWGPGAAKSAVGTAILLELVRT

SEQ ID NO: 805 - protein, 25 aa, Homo sapiens
EAFSHFTKIITPAITRVVDFAKKLP

SEQ ID NO: 806 - protein, 25 aa, Homo sapiens
EGQSQQFSVSENLLKEAIRAIFPSR

SEQ ID NO: 807 - protein, 25 aa, Homo sapiens
PASLGKWKKEPELAAFVFKTAVVLV

SEQ ID NO: 808 - protein, 25 aa, Homo sapiens
RLPSSWALFSPLLAGLALLGVGPVP

SEQ ID NO: 809 - protein, 25 aa, Homo sapiens
AIKKKLVQRLEHAAKQAAASATQTI

SEQ ID NO: 810 - protein, 25 aa, Homo sapiens
GQLLRGVGAAATAVTQALNELLQHV

SEQ ID NO: 811 - protein, 25 aa, Homo sapiens
QQLAAFSKRVAGAVTELIQAAEAMK

SEQ ID NO: 812 - protein, 25 aa, Homo sapiens
SELLKQVSAAASVVSQALHDLLQHV

SEQ ID NO: 813 - protein, 25 aa, Homo sapiens
KVLNLAYNKINKIADEAFYGLDNLQ

SEQ ID NO: 814 - protein, 25 aa, Homo sapiens
GREKFKSRGVGELARLALVISELEG

SEQ ID NO: 815 - protein, 25 aa, Homo sapiens
QGHQFLREREEHLLEQLAKLEQELT

SEQ ID NO: 816 - protein, 25 aa, Homo sapiens
LKISPVAPDADAVAAQILSLLPLKF

SEQ ID NO: 817 - protein, 25 aa, Homo sapiens
RKARGYLRLVPLFVLLALLVLASAG

SEQ ID NO: 818 - protein, 25 aa, Homo sapiens
YAHPQQKVAVYRALQAALAESGGSP

SEQ ID NO: 819 - protein, 25 aa, Homo sapiens
YVNYVLSEKSSTFLMKAAAKVVESK

SEQ ID NO: 820 - protein, 25 aa, Homo sapiens
AAPAPGAPLLPLLLPALAARLLPPA

SEQ ID NO: 821 - protein, 25 aa, Homo sapiens
THIKAPEQQVKNILNELFQRENRVL

SEQ ID NO: 822 - protein, 25 aa, Homo sapiens
PHMRKNIKGIHTLLQNLAKASPVYL

SEQ ID NO: 823 - protein, 25 aa, Homo sapiens
QHALRNRRLLRKVIKAAALEEHNAA

SEQ ID NO: 824 - protein, 25 aa, Homo sapiens
KSDRLISLQSASVVYDLLTIALGRR

SEQ ID NO: 825 - protein, 25 aa, Homo sapiens
QMKAKRTKEAVEVLKKALDAISHSD

SEQ ID NO: 826 - protein, 25 aa, Homo sapiens
LARQINHPELHMVLSNLAAVLMHRE

SEQ ID NO: 827 - protein, 25 aa, Homo sapiens
VAPHGRGPGLLPLLAALAWFSRPAA

SEQ ID NO: 828 - protein, 25 aa, Homo sapiens
HSRTSWVPVVLGVLTALVTAAALAL

SEQ ID NO: 829 - protein, 25 aa, Homo sapiens
PLPLPPPPRLGLLLAALASLLLPES

SEQ ID NO: 830 - protein, 25 aa, Homo sapiens
WSWLLGAAMVGAVLTALLAGLVSLL

SEQ ID NO: 831 - protein, 25 aa, Homo sapiens
LLHDDRGPVLEALVARAIRNIEMTQ

SEQ ID NO: 832 - protein, 25 aa, Homo sapiens
RGGGQIIPTARRVVYSAFLMATPRL

SEQ ID NO: 833 - protein, 25 aa, Homo sapiens
LFYQDKLKSLHQLLEVLLALLDKDV

SEQ ID NO: 834 - protein, 25 aa, Homo sapiens
YGSGPKRFPLVDVLQYALEFASSKP

SEQ ID NO: 835 - protein, 25 aa, Homo sapiens
NRKFVDPSAALDLLKGAFRSSEEQQ

SEQ ID NO: 836 - protein, 25 aa, Homo sapiens
KIEKVFSKLLYPIVRGAALSVLKYM

SEQ ID NO: 837 - protein, 25 aa, Homo sapiens
RFLNLLMNDAIFLLDEAIQYLSKIK

SEQ ID NO: 838 - protein, 25 aa, Homo sapiens
DIPSADRHKSKLIAGKIIPAIATTT

SEQ ID NO: 839 - protein, 25 aa, Homo sapiens
GIPPVNRAQSKRIVGQIIPAIATTT

SEQ ID NO: 840 - protein, 25 aa, Homo sapiens
PVDKVAAMREFRVLHTALHSSSSYR

SEQ ID NO: 841 - protein, 25 aa, Homo sapiens
AARFAKTKEEVEAAKAAALLAKQAE

SEQ ID NO: 842 - protein, 25 aa, Homo sapiens
HLPEKQDTFAEKLVTQIIKNLQVKI

SEQ ID NO: 843 - protein, 25 aa, Homo sapiens
MLNRQDPFTVHMAARIIAKLAAWGK

SEQ ID NO: 844 - protein, 25 aa, Homo sapiens
ALIEGKNKTVSTLVIQAANVSALYK

SEQ ID NO: 845 - protein, 25 aa, Homo sapiens
EVITDNLPGSIRAVVNIFLVAKALL

SEQ ID NO: 846 - protein, 25 aa, Homo sapiens
VRWLQESRRSRKLILFIVFLALLLD

SEQ ID NO: 847 - protein, 25 aa, Homo sapiens
FVIATTRQRLFQFIGRAAEGAEAQG

SEQ ID NO: 848 - protein, 25 aa, Homo sapiens
WIEMGSRLDARQLIPALVNYSQGGE

SEQ ID NO: 849 - protein, 25 aa, Homo sapiens
MRDVNKKFSVRYFLNLVLVDEEDRR

SEQ ID NO: 850 - protein, 25 aa, Homo sapiens
NSFKMKMSVILGIIHMLFGVSLSLF

SEQ ID NO: 851 - protein, 25 aa, Homo sapiens
NSYKMKMSVILGIVQMVFGVILSLF

SEQ ID NO: 852 - protein, 25 aa, Homo sapiens
MQREGGPSQIGDALGFAVRYLTSEM

SEQ ID NO: 853 - protein, 25 aa, Homo sapiens
KDTPSGISKVRKILFTLAKQSKALG

SEQ ID NO: 854 - protein, 25 aa, Homo sapiens
LVGLKNGQILKIFVDNLFAIVLLKQ

SEQ ID NO: 855 - protein, 25 aa, Homo sapiens
SFEQGGSEFVPVVVSRLVLRSMSRR

SEQ ID NO: 856 - protein, 25 aa, Homo sapiens
IPADPEAGGIGRVVNGAFMVLKGHR

SEQ ID NO: 857 - protein, 25 aa, Homo sapiens
RQNKDERYSIKDLLNHAFFQEETGV

SEQ ID NO: 858 - protein, 25 aa, Homo sapiens
RKTSKSKLKAGKLLNPLVRQLKVVA

SEQ ID NO: 859 - protein, 25 aa, Homo sapiens
RTDKNERFTIQDLLAHAFFREERGV

SEQ ID NO: 860 - protein, 25 aa, Homo sapiens
TAFGADTEGSQWIIGYLLQKVISNL

SEQ ID NO: 861 - protein, 25 aa, Homo sapiens
LGLNDETMVLSVFIGKIITNLKYWG

SEQ ID NO: 862 - protein, 25 aa, Homo sapiens
LNYLATRPKLATFVTQALIQLYARI

SEQ ID NO: 863 - protein, 25 aa, Homo sapiens
AMFVEYRKQLKLLLDRLAQVSPELL

SEQ ID NO: 864 - protein, 25 aa, Homo sapiens
QNWQTTRFMEVEVAIRLLYMLAEAL

SEQ ID NO: 865 - protein, 25 aa, Homo sapiens
FPAATSGRMVEAFARRALWDAGLNY

SEQ ID NO: 866 - protein, 25 aa, Homo sapiens
LLKASHVRDAVAVIRYLVWLEKNVP

SEQ ID NO: 867 - protein, 25 aa, Homo sapiens
SILTQPHLYSPVLISQLVQMASQLR

SEQ ID NO: 868 - protein, 25 aa, Homo sapiens
ISIGTIYFRAHKLVLAAASLLFKTL

SEQ ID NO: 869 - protein, 25 aa, Homo sapiens
RDVLRVQGVSLTALRLLLADAYSGR

SEQ ID NO: 870 - protein, 25 aa, Homo sapiens
QLEGLENATARNLLGKLINILLAVM

SEQ ID NO: 871 - protein, 25 aa, Homo sapiens
PPLIVDRERLKKLLDLLVDKSNNLA

SEQ ID NO: 872 - protein, 25 aa, Homo sapiens
EDTKEKRTIIHQAIKSLFPGLETKT

SEQ ID NO: 873 - protein, 25 aa, Homo sapiens
DQKTNLPEYLQTLLNTLAPLLLFRA

SEQ ID NO: 874 - protein, 25 aa, Homo sapiens
RSREHGTLWSLIIAKLILSRSISSD

SEQ ID NO: 875 - protein, 25 aa, Homo sapiens
VLASAYTGRLSMAAADIVNFLTVGS

SEQ ID NO: 876 - protein, 25 aa, Homo sapiens
GRNKMDPPRSSIFLQEVITTVYGYK

SEQ ID NO: 877 - protein, 25 aa, Homo sapiens
VDLAYSNYHVKQFLEALLRNSAAPS

SEQ ID NO: 878 - protein, 25 aa, Homo sapiens
LKLRSLRVNVGQLLAMIVPDLDLQQ

SEQ ID NO: 879 - protein, 25 aa, Homo sapiens
ELQKQAELMEFEIALKALSVLRYIT

SEQ ID NO: 880 - protein, 25 aa, Homo sapiens
DALHMLTDLSAIILTLLALWLSSKS

SEQ ID NO: 881 - protein, 25 aa, Homo sapiens
MDDEENYSAASKAVRQVLHQLKRLG

(19) United States
(12) Patent Application Publication
(10) Pub. No.: US 2009/0104177 A1
(43) Pub. Date: Apr. 23, 2009

(54) PEPTIDES FOR INHIBITING THE INTERACTION OF PROTEIN KINASE A AND PROTEIN KINASE A ANCHOR PROTEINS

(75) Inventors: Enno Klussmann, Berlin (DE); Walter Rosenthal, Kleinmachnow (DE); Christian Hundsrucker, Berlin (DE)

Correspondence Address: JOYCE VON NATZMER;PEQUIGNOT + MYERS LLC, 200 Madison Avenue, Suite 1901, New York, NY, 10016, US

(73) Assignee: FORSCHUNGSVERBUND BERLIN E.V., Berlin (DE)

(21) Appl. No.: 11/571,117

(22) PCT Filed: Jun. 29, 2005
(86) PCT No.: PCT/DE2005/001181

§ 371 Date: Dec. 8, 2008

(30) Foreign Application Priority Data
Jun. 29, 2004 (DE) 10 2004 031 579.5

(51) Int. Cl. A61K 39/395; C07K 14/00; C12N 15/11; C12N 15/00; C12N 5/06; A01K 67/027; C12N 9/99; A61K 31/7105; C07K 16/18; A61K 31/7088; A61K 38/00; A61K 35/12

(52) U.S. Cl. 424/130.1; 530/324; 536/23.1; 435/320.1; 435/352; 800/13; 800/18; 800/14; 530/387.1; 536/24.5; 514/44; 514/12; 424/93.21; 435/184

(57) ABSTRACT

The invention relates to a nucleic acid sequence encoding peptides which inhibit the interaction of protein kinase A (PKA) and protein kinase A anchor proteins (AKAP), to a host organism comprising said nucleic acid sequence and optionally expressing said peptides, to the use of said peptides and of said host organism in investigating diseases associated with said AKAP-PKA interaction, and to the use of said peptides as pharmaceutical agent for the treatment of such diseases.

The invention relates to nucleic acid sequences encoding peptides which inhibit the interaction of protein kinase A (PKA) and protein kinase A anchor proteins (AKAP), to a host organism comprising said nucleic acid sequences and expressing the peptides of the invention, to the use of said peptides and of said host organism in therapy and experimental investigation of diseases associated with a modified AKAP-PKA interaction, and to the use of said peptides as pharmaceutical agents for the treatment of such diseases, specifically insipid diabetes, duodenal ulcer, hypertony and pancreatic diabetes.

The biological activity of hormones and neurotransmitters is mediated via activation of signal cascades altering the phosphorylation state of effector proteins. Two classes of enzymes are involved in this reversible process: protein kinases and phosphoprotein phosphatases. Phosphorylation is effected by kinases catalyzing the transfer of the terminal phosphate group of ATP on specific serine or threonine residues, and dephosphorylation is mediated by phosphoprotein phosphatases. One mechanism of controlling and regulating such enzyme activities is compartmentation of these enzymes by association with anchor proteins located near their substrates. Protein kinase A (PKA) is one of the multifunctional kinases with broad substrate specificity, which is anchored on subcellular structures by so-called protein kinase A anchoring proteins (AKAPs).

In many essential cellular processes such as contraction, secretion, metabolism, gene transcription, cell growth and division, the transduction of extracellular signals proceeds via G protein-coupled receptors, G protein Gₛ, activation of an adenyl cyclase, and formation of the second messenger cyclic adenosine monophosphate (cAMP). The effects of cAMP are mediated by the cAMP-dependent PKA.

The protein kinase A (PKA) holoenzyme consists of a dimer of regulatory (R) subunits, each of which has a catalytic (C) subunit bound thereto. Activation of the kinase by binding of two cAMP molecules to each R subunit induces dissociation of the C subunits which phosphorylate substrates in the proximity thereof. Corresponding to the existence of type I (RI) or type II (RII) regulatory subunits, the PKA holoenzyme is referred to as type I or type II PKA. The RI subunits have RIα and RIβ, the RII subunits have RIIα and RIIβ and the C subunits Cα, Cβ and Cγ. The different PKA subunits are encoded by different genes (Klussmann, 2004; Tasken and Aandahl, 2004).

The regulatory subunits show varying expression patterns. While RIα and RIIα are ubiquitous in tissues, the regulatory subunit RIβ is predominantly found in the brain.

Association of the two R subunits with intracellular compartments is mediated by AKAPs. The anchor proteins are a group of functionally related molecules characterized by the interaction with type I or type II of the regulatory subunits (RI and RII, respectively) of the PKA holoenzyme. The first anchor proteins have been isolated during affinity-chromatographic purification of the R subunits on cAMP-Sepharose. These associated proteins showed RII binding even after transfer onto a nitrocellulose membrane. This observation also forms the basis of the most common method (RII overlay) of detecting AKAPs. It is a modified Western blot wherein radioactively labelled RII subunits rather than a primary antibody are used as probe.

To date, little is known about the functional significance of the RI-AKAP interaction. Although RIα is mainly found in the cytosol, a number of studies show anchoring in vivo. Dynamic anchoring of the RIα subunits—as opposed to static anchoring of RII subunits—seems to be of crucial significance to the cell. Thus, association of the RI sub-units with the plasma membrane of erythrocytes and activated T lymphocytes has been described. In cAMP-mediated inhibition of T cell proliferation by type I PKA, localization of the enzyme possibly could be mediated by AKAPs. In knockout mice, which do not express any regulatory type II subunits in their skeletal muscle tissue, the RIα subunits bind to a calcium channel-associated AKAP, thereby obtaining normal, cAMP-dependent channel conductivity as a result of the proper availability of the catalytic subunits of PKA.

Furthermore, it has been shown in vivo that the catalytic subunits in the cell preferentially associate with the RII subunits, and that type I PKA holoenzyme is formed when the amount of free catalytic subunits exceeds the amount of free RII subunits.

Specificity in PKA anchoring is achieved by virtue of the targeting domain—a structural motif which, in contrast to the anchoring domain, is neither conserved in the sequence, nor in the structure of the AKAPs. Thus, AKAPs are anchored to structural elements in the cell by protein-protein interactions and to membranes by protein-lipid interactions.

The literature describes various AKAPs undergoing association with various cellular compartments, for instance with the centrosomes, mitochondria, the endoplasmic reticulum and Golgi apparatus, the plasma and nuclear membranes, and vesicles.

To date, the precise mechanisms of anchoring are known for only a few AKAPs. Thus, the myocardium-specific anchor protein mAKAP is anchored to the perinuclear membrane of the cardiomyocytes by a region including three spectrin-like repeat sequences. Two isoforms of AKAP15/18 are anchored to the plasma membrane via lipid modifications (myristoylation and palmitoylation). Three polybasic regions in the targeting domain of AKAP79 are involved in the localization of the protein on the inner postsynaptic membrane (PSD, post-synaptic density).

AKAPs were first characterized via the interaction with PKA. However, some of these proteins may also bind other enzymes involved in signal transduction.

As a result of simultaneous anchoring of enzymes catalyzing opposing reactions, such as kinases and phosphatases, these AKAPs—also referred to as scaffolding proteins—can localize entire signal complexes in the vicinity of particular substrates, thereby contributing to the specificity and regulation of the cellular response to extracellular signals. AKAP79 was the first AKAP where interaction with a plurality of enzymes could be detected. Said protein binds protein kinase A, protein kinase C and the protein phosphatase calcineurin (PP2B), each enzyme being inhibited in bound condition. Distinct signals are required for the activation of each individual enzyme, which is why various second messengers such as cAMP, calcium and phospholipids may be present together at this position. Further examples are AKAP220, which localizes PKA and protein phosphatase PP1 on the peroxisomes, and the yotiao AKAP which, in addition to PKA, also binds protein phosphatase PP1. The CG-NAP AKAP not only binds PKA and protein phosphatase PP1, but also the rho-dependent kinase PKN (NGF (nerve growth factor)-activated protein kinase) and protein phosphatase PP2A.

Other proteins may also undergo association with AKAPs. Thus, ezrin, a member of the cytoskeleton-associated ERM family ezrin, radixin and moesin, which has been identified as an AKAP, binds to a protein (EBP50/NHERF) which is involved in the regulation of the sodium-proton transport in the apical membrane of epithelial cells. AKAPs mediate the modulation of the conductivity of ion channels by localization of protein kinases and phosphatases in the vicinity of particular channel subunits probably regulated by phosphorylation and dephosphorylation.

The activity of the NMDA receptor is modulated by the yotiao AKAP which also binds protein phosphatase PP1. The phosphatase, which is active in bound condition, limits the channel conductivity of the NMDA receptor until the PKA is activated by cAMP, phosphorylating the ion channel or an associated protein so that the conductivity rapidly increases. It has also been shown that myristoylated Ht31 peptides inhibiting the interaction between PKA and AKAP suspend the cAMP-dependent inhibition of interleukin-2 transcription in Jurkat T cells, and that S-Ht31 peptides restrict sperm motility.

AKAPs are also involved in essential complex biological processes, such as insulin secretion in β-cells of the pancreas and in RINm5F cells (clonal β-cell line of rats) mediated by the hormone GLP-1 (glucagon-like peptide). The activation of PKA by GLP-1 results in phosphorylation of L-type calcium channels, favoring exocytosis of insulin from secretory granules. Ht31 peptide-mediated inhibition of PKA anchoring results in a significant reduction of insulin secretion. Said peptides neither affect cAMP formation nor the activity of the catalytic subunits of PKA. Furthermore, an increase in insulin secretion after application of GLP-1 could be detected following expression of wild-type AKAP18α in RINm5F cells compared to control cells failing to express AKAP18α.

The redistribution of the aquaporin-2 water channel from intracellular vesicles to the plasma membrane of the principal cells of the renal collecting tubule, mediated by the antidiuretic hormone arginine-vasopressin (AVP), the molecular basis of the vasopressin-mediated water reabsorption, is another example of a process requiring interaction of the PKAs with AKAP proteins (Klussmann et al., 1999). If the interaction is prevented, redistribution cannot occur. However, the interaction also plays an important role in many processes in a wide variety of cell types; for example, the interaction increases the myocardial contractility (Hulme et al., 2003).

To analyze the effect of PKA-AKAP interaction, efficient and selective modification of the interaction, especially inhibition or decoupling, is required. At present, an Ht31 peptide is available for decoupling of the PKAs from AKAP proteins. The Ht31 peptide can be coupled to stearate so as to be present in a membrane-permeable form. However, the Ht31 peptide decouples PKA and AKAP in a way which is insufficient for many investigations or even therapeutic use. Above all, the Ht31 peptide fails to undergo selective interaction with the regulatory subunits RIIα or RIIβ of PKAs, so that the significance of the subunits for selected processes cannot be analyzed.

The object of the invention is therefore to overcome the above-mentioned drawbacks and, in particular, provide new nucleic acid sequences which encode peptides modifying, particularly decoupling, the interaction of AKAP and PKA in an efficient and specific way and, in addition, can be used as overexpressing materials in host organisms to perform model analyses with the aid of these host organisms, e.g. mice, of diseases associated with an AKAP-PKA interaction, preferably insipid diabetes, duodenal ulcer, hypertony and pancreatic diabetes.

The present invention solves the above technical problem by providing an isolated nucleic acid sequence selected from the group comprising:
a) a nucleic acid molecule comprising a nucleotide sequence encoding at least one amino acid sequence selected from the group comprising SEQ ID Nos. 1-39,
b) a nucleic acid molecule which undergoes hybridization with a nucleotide sequence according to a) under stringent conditions,
c) a nucleic acid molecule comprising a nucleotide sequence having sufficient homology to be functionally analogous to a nucleotide sequence according to a) or b),
d) a nucleic acid molecule which, as a consequence of the genetic code, is degenerated into a nucleotide sequence according to a)-c), and/or
e) a nucleic acid molecule in accordance with a nucleotide sequence according to a)-d), which is modified and functionally analogous to a nucleotide sequence according to a)-d) as a result of deletions, additions, substitutions, translocations, inversions and/or insertions.

Surprisingly, the nucleic acid sequences according to the invention can be used to encode peptides in accordance with Table 1 (SEQ ID Nos. 1-39) which modify, preferably inhibit, and more preferably decouple the interaction of AKAP and PKA. The nucleic acid molecules according to the invention are advantageously suited to encode peptides binding selectively to regulatory subunits of the PKAs, especially to RIIα or RIIβ. Furthermore, the peptides encoded by the nucleic acid molecules according to the invention offer a way of effecting modification, inhibition or decoupling of AKAP and PKA in dependence of the species being used. The nucleic acid molecules or the peptides derived therefrom are advantageously suited to produce transgenic organisms, e.g. mice, in which the AKAP-PKA interaction is modified in a tissue- and/or cell-specific fashion.

In a preferred embodiment of the invention the nucleic acid sequence having sufficient homology to be functionally analogous to a nucleotide sequence has at least 40% homology. In the meaning of the invention, functional analogy to the above-mentioned nucleic acid sequences or to sequences hybridizing with said nucleic acid sequences implies that the encoded homologous structures allow efficient and selective decoupling of the PKA-AKAP interaction and have high affinity in binding to RII subunits of PKA.

In another advantageous embodiment of the invention, the nucleic acid molecule has at least 60%, preferably 70%, more preferably 80%, and most preferably 90% homology to the nucleic acid molecules according to the invention.

In another preferred embodiment of the invention, the nucleic acid molecule is a genomic DNA and/or an RNA, and in a particularly preferred fashion the nucleic acid molecule is a cDNA.

The invention also relates to a vector comprising at least one nucleic acid molecule according to the invention. Further, the invention relates to a host cell comprising said vector. The invention also relates to a polypeptide encoded by at least one nucleic acid molecule according to the invention.

In a preferred embodiment of the invention the polypeptide comprises an amino acid sequence according to SEQ ID NO. 1 to SEQ ID NO. 39 or at least one polypeptide in accordance with these sequences. The invention also relates to a polypeptide which has been modified by deletion, addition, substitution, translocation, inversion and/or insertion and is functionally analogous to a polypeptide according to SEQ ID Nos. 1 to 39 and/or a polypeptide comprising a polypeptide which has sufficient homology to be functionally analogous to a polypeptide according to SEQ ID Nos. 1 to 39 or mutations thereof (deletion, addition, substitution, translocation, inversion and/or insertions).

The following peptides of the invention are particularly preferred:

| | |
|---|---|
| SEQ ID NO. 1 | PEDAELVRLSKRLVENAVLKAVQQY |
| | (Akap18delta-wt) |
| SEQ ID NO. 2 | PEDAELVRTSKRLVENAVLKAVQQY |
| | (AKAP18delta-L304T) |
| SEQ ID NO. 3 | PEDAELVRLSKRDVENAVLKAVQQY |
| | (AKAP18delta-L308D) |
| SEQ ID NO. 4 | PEDAELVRLSKRLVENAVEKAVQQY |
| | (AKAP18delta-L314E) |
| SEQ ID NO. 5 | PEDAELVRLSKRLPENAVLKAVQQY |
| | (AKAP18delta-P) |
| SEQ ID NO. 6 | PEDAELVRLSKRLPENAPLKAVQQY |
| | (AKAP18delta-PP) |
| SEQ ID NO. 7 | PEDAELVRLDKRLPENAPLKAVQQY |
| | (AKAP18delta-phos) |
| SEQ ID NO. 8 | EPEDAELVRLSKRLVENAVLKAVQQYLEETQ |
| | (Akap18delta-RI) |
| SEQ ID NO. 9 | NTDEAQEELAWKIAKMIVSDIMQQA |
| SEQ ID NO. 10 | VNLDKKAVLAEKIVAEAIEKAEREL |
| SEQ ID NO. 11 | NGILELETKSSKLVQNIIQTAVDQF |
| SEQ ID NO. 12 | TQDKNYEDELTQVALALVEDVINYA |
| SEQ ID NO. 13 | LVDDPLEYQAGLLVQNAIQQAIAEQ |
| SEQ ID NO. 14 | QYETLLIETASSLVKNAIQLSIEQL |
| SEQ ID NO. 15 | LEKQYQEQLEEEVAKVIVSMSIAFA |
| SEQ ID NO. 16 | EEGLDRNEEIKRAAFQIISQVISEA |
| SEQ ID NO. 17 | ETSAKDNINIEEAARFLVEKILVNH |
| SEQ ID NO. 18 | ADRGSPALSSEALVRVLVLDANDNS |
| SEQ ID NO. 19 | SDRGSPALSSEALVRVLVLDANDNS |
| SEQ ID NO. 20 | TDRGFPALSSEALVRVLVLDANDNS |
| SEQ ID NO. 21 | FLAGETESLADIVLWGALYPLLQDP |
| SEQ ID NO. 22 | SELLKQVSAAASWSQALHDLLQHV |
| SEQ ID NO. 23 | EKESLTEEEATEFLKQILNGVYYLH |
| SEQ ID NO. 24 | EKGYYSERDAADAVKQILEAVAYLH |
| SEQ ID NO. 25 | WLYLQDQNKAADAVGEILLSLSYLP |
| SEQ ID NO. 26 | LKISPVAPDADAVAAQILSLLPLKF |
| SEQ ID NO. 27 | SKTEQPAALALDLVNKLVYWVDLYL |
| SEQ ID NO. 28 | VLASAYTGRLSMAAADIVNFLTVGS |
| SEQ ID NO. 29 | VKLSNLSNLSHDLVQEAIDHAQDLQ |
| SEQ ID NO. 30 | APSDPDAVSAEEALKYLLHLVDVNE |
| SEQ ID NO. 31 | QMKAKRTKEAVEVLKKALDAISHSD |
| SEQ ID NO. 32 | KDKLKPGAAEDDLVLEWIMIGTVS |
| SEQ ID NO. 33 | EKRVADPTLEKYVLSWLDTINAFF |
| SEQ ID NO. 34 | QENLSLIGVANVFLESLFYDVKLQY |
| SEQ ID NO. 35 | HQSWYRKQAAMILNELVTGAAGLE |
| SEQ ID NO. 36 | QQLQKQLKEAEQILATAVYQAKEKL |
| SEQ ID NO. 37 | HSVMDTLAVALRVAEEAIEEAISKA |
| SEQ ID NO. 38 | RQVQETLNLEPDVAQHLLAHSHWGA |
| SEQ ID NO. 39 | DIPSADRHKSKLIAGKIIPAIATTT |

The peptides of the invention are derived either (i) from AKAP18δ (SEQ ID Nos. 1 to 7) or (ii) from proteins not associated with AKAP molecules (SEQ ID Nos. 8 to 39).

The peptides according to (i):

### AKAP18δ-wt

### AKAP18δ-L304T

### AKAP18δ-L314E

### AKAP18δ-RI

have in common that the RIIα subunits of the PKA bind stronger than any other peptide derived from natural AKAPs. We explain this by binding via hydrogen bridges (H bridges) between peptide and RII dimer (see Fig., hydrogen bridges represented by broken lines). Correspondingly, a common feature of the peptides is the minimum number (8) of amino acids forming H bridges.

The following peptides are also derived from AKPA18δ, but involve the feature of absent binding of RII subunits of the PKAs despite high similarity of the amino acids (negative controls; if necessary, patenting can be renounced). They have in common that binding is no longer present due to structural differences (1, 2) or differences in charge (3, 4).

### 1 AKAP18δ-P

### 2 AKAP18δ-PP

### 3 AKAP18δ-L308D

4 AKAP18δ-phos

The peptides according to the invention derived from proteins other than AKAPs have a well-defined size which, surprisingly, contributes to the ability of the peptides of modifying the interaction between AKAP and PKA because it has an influence on the affinity of the peptides to the RIIα subunits of the PKAs. The peptides are constituted of 25 amino acids and are therefore 25 mers.

Selecting the peptides so as to be shorter or longer (e.g. 17 mers) will change their activity. The common structural feature of peptide length, together with the functional feature of AKAP/PKA decoupling, defines the structures according to the invention. The peptides according to the invention are characterized by the general formula:

xxxxxxxxx[AVLISE]xx[AVLIF][AVLI]xx[AVLI][AVLIF]xx
[AVLISE]xxxx

wherein x represents an arbitrary amino acid, and x more specifically represents any of the 20 biogenic amino acids (in the single-letter code, these are: A, C, D, E, F, G, H, I, K, L, M, N, P, Q, R, S, T, V, W, Y). Each amino acid disclosed in Alberts et al. (2004), Molekularbiologie der Zelle, pp. 8, 73, 79ff, 150ff, or 1717G; in Römpp (1999), Biotechnologie und Gentechnik, pp. 45ff, or in Römpp (2000), Lexikon Biochemie und Molekularbiologie, pp. 28ff, or in other standard textbooks of biology is claimed herein. These particularly preferred peptides have either a positively charged amino acid (H, K or R) in the first or second position (position is the number of the amino acid from the N terminus) or leucine in the positions 19, 18 or 14 or serine in position 4.

A functionally analogous peptide is a peptide which is capable of modifying, preferably decoupling, the PKA-AKAP interaction.

The invention also relates to an organism overexpressing a nucleic acid molecule of the invention or comprising a vector of the invention and/or having a polypeptide according to the invention. For example, this can be a transgenic mouse or rat, or cattle, horse, donkey, sheep, camel, goat, pig, rabbit, guinea pig, hamster, cat, monkey or dog in which tissue- and/or cell-specific disorders of the PKA-AKAP interaction are present. In particular, such organisms, for example mice, can be used to develop pharmaceutical agents which modify, preferably decouple, the PKA-AKAP interaction.

The organisms of the invention also allow in vivo investigations of metabolic processes where PKA-AKAP interaction plays a role, or which processes require clarification as to whether AKAP-PKA interaction is involved in a particular incident.

Preferably, the organism is a transgenic mouse overexpressing the strongly binding peptide AKAP18δ-L304T or AKAP18δ-L314E specifically in the principal cells of the renal collecting tubules. Advantageously, decoupling of the PKAs from the AKAP proteins results in prevention of the vasopressin-induced redistribution of AQP2 in primarily cultured cells of the collecting tubule, so that the animals exhibit insipid diabetes, in particular. This disease is remarkable for a massive loss of water (polyuria) which e.g. human patients attempt to compensate by ingestion of large amounts of liquid (polydipsia).

For example, the transgenic organisms according to the invention allow investigations as to what extent decoupling of PKAs or of selected subunits of AKAP proteins can be regarded as a therapeutic principle and put to use. Advantageously, such investigations can be followed by analysis of optimized substances (pharmaceutical agents) having the same effect. Substances optimized in this way preferably have an aquaretic effect and can therefore be used with advantage in patients with edemas, e.g. in cases of cardiac failure or liver cirrhosis.

The invention also relates to a recognition molecule directed against said nucleic acid molecule, said vector, said host cell, and/or said polypeptide. Recognition sub-stances in the meaning of the invention are molecules capable of interacting with the above-mentioned structures such as nucleic acid molecules or sequences, vectors, host cells and/or polypeptides or fragments thereof, particularly interacting in such a way that detection of said structures is possible. In particular, said recognition substances can be specific nucleic acids binding to the above-mentioned nucleic acid molecules or polypeptides, such as antisense constructs, cDNA or mRNA molecules or fragments thereof, but also antibodies, fluorescent markers, labelled carbohydrates or lipids or chelating agents. Of course, it is also possible that the recognition substances are not proteins or nucleic acids or antibodies, but instead, antibodies directed against the same. In this event, the recognition substances can be secondary antibodies, in particular.

In a special embodiment of the invention, the recognition molecule is an antibody, an antibody fragment and/or an antisense construct, especially an RNA interference molecule.

The antibodies in the meaning of the invention bind the polypeptides in a specific manner. The antibodies may also be modified antibodies (e.g. oligomeric, reduced, oxidized and labelled antibodies). The term “antibody” used in the present specification includes intact molecules, as well as antibody fragments such as Fab, F(ab′)₂ and Fv capable of binding the particular epitope determinants of the polypeptides. In these fragments, the antibody's ability of selectively binding its antigen or receptor is partially retained, the fragments being defined as follows:
(1) Fab: this fragment which includes a monovalent antigen-binding fragment of an antibody molecule can be produced by cleavage of a complete antibody using the enzyme papain, obtaining an intact light chain and part of a heavy chain being;
(2) the Fab′ fragment of an antibody molecule can be produced by treatment of a complete antibody with pepsin and subsequent reduction, resulting in an intact light chain and part of a heavy chain; two Fab′ fragments per antibody molecule are obtained;
(3) F(ab′)₂: fragment of the antibody which can be obtained by treatment of a complete antibody with the enzyme pepsin with no subsequent reduction; F(ab′)₂ is a dimer of two Fab′ fragments held together by two disulfide bonds;
(4) Fv: defined as a fragment modified by genetic engineering, which includes the variable region of the light chain and the variable region of the heavy chain and is expressed in the form of two chains; and
(5) single-chain antibodies (“SCA”), defined as a molecule modified by genetic engineering, which includes the variable region of the light chain and the variable region of the heavy chain, which regions are linked by means of a suitable polypeptide linker to form a genetically fused single-chain molecule.

The invention also relates to a pharmaceutical composition comprising said nucleic acid molecule of the invention, said vector of the invention, said host cell of the invention, said polypeptide of the invention and/or said recognition molecule of the invention, optionally together with a pharmaceutically acceptable carrier.

In a preferred embodiment of the invention the pharmaceutical composition is an aquaretic agent. Aquaretic agents in the meaning of the invention modify the interaction between PKAs and AKAP proteins; more specifically, they decouple the interaction between the two mentioned above. It will be appreciated that the recognition molecules of the invention can also be used as pharmaceutical compositions, especially those directed against the peptide according to the invention or against the coding nucleic acid.

In particular, the pharmaceutical compositions comprising the peptides of the invention, the vectors of the invention or the recognition molecules of the invention can be used in patients with edemas, particularly in cases of cardiac failure or liver cirrhosis. In the meaning of the invention, the vectors or the nucleic acid molecules of the invention can be employed as pharmaceutical composition on a nucleic acid level, whereas the peptides according to the invention, but also part of the recognition molecules of the invention, can be used on an amino acid level. Depending on whether the therapy consists in decoupling of AKAP and PKA—e.g. by means of the peptides according to the invention—or in preventing decoupling between AKAP and PKA—e.g. by means of the antibodies of the invention directed against said peptides—the peptides of the invention or the recognition molecules of the invention directed e.g. against said peptides or other structures can preferably be used as pharmaceutical composition by a person skilled in the art. In particular, the peptides of the invention can be used in decoupling of AKAP/PKA and thus in case of edemas. The recognition molecules of the invention (e.g. antibodies) are particularly useful in preventing de-coupling of AKAP/PKA, e.g. in cases of insipid diabetes.

Of course, the peptides according to the invention may also comprise conventional auxiliaries, preferably carriers, adjuvants and/or vehicles. For example, the carriers can be fillers, diluents, binders, humectants, disintegrants, dissolution retarders, absorption enhancers, wetting agents, adsorbents and/or lubricants. In this event, the peptide is specifically referred to as drug or pharmaceutical agent.

In another preferred embodiment of the invention the agent according to the invention is formulated as a gel, poudrage, powder, tablet, sustained-release tablet, premix, emulsion, brew-up formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can also be composed such that release of the active substance(s) takes place only or preferably in a particular area of the intestinal tract, optionally in a delayed fashion, to which end polymer sub-stances and waxes can be used as embedding materials.

For example, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate are also added. For oral administration in the form of capsules, diluents that can be used include lactose and dried corn starch. In oral administration of aqueous suspensions the active substance is combined with emulsifiers and suspending agents. Also, particular sweeteners and/or flavors and/or coloring agents can be added, if desired.

The active substance(s) can also be present in micro-encapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substances CHP and gemcitabine, solutions and emulsions may include conventional carriers such as solvents, solubilizers and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substances, suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar, and tragacanth, or mixtures of these substances.

The drugs can be present in the form of a sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their poly-oxyethylated forms. Such oil solutions or suspensions may also include a long-chain alcohol or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the peptides according to the invention should be present in the above-mentioned pharmaceutical preparations at a concentration of about 0.01 to 99.9 wt.-%, more preferably about 0.05 to 99 wt.-% of the overall mixture.

In addition the peptides or structural homologs, e.g. peptides with D-amino acids, or functional analogs, e.g. peptide mimetics, the above-mentioned pharmaceutical preparations may include further pharmaceutical active substances. The production of the pharmaceutical preparations specified above proceeds in a usual manner according to well-known methods, e.g. by mixing the active substance(s) with the carrier material(s).

The above-mentioned preparations can be applied in humans and animals on an oral, rectal, parenteral (intravenous, intramuscular, subcutaneous), intracisternal, intravaginal, intraperitoneal route, locally (powders, ointment, drops) and used in the therapy of tumors. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Moreover, the drugs or combined agents can be incorporated in other carrier materials such as plastics (plastic chains for local therapy), collagen or bone cement.

In another preferred embodiment of the invention, the peptides are incorporated in a pharmaceutical preparation at a concentration of 0.1 to 99.5, preferably 0.5 to 95, and more preferably 20 to 80 wt.-%. That is, the peptides are present in the above-specified pharmaceutical preparations, e.g. tablets, pills, granulates and others, at a concentration of preferably 0.1 to 99.5 wt.-% of the overall mixture. Those skilled in the art will be aware of the fact that the amount of active substance, i.e., the amount of an inventive compound combined with the carrier materials to produce a single dosage form, will vary depending on the patient to be treated and on the particular type of administration. Once the condition of a patient has improved, the proportion of active compound in the preparation can be modified so as to obtain a maintenance dose that will bring the disease to a halt. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents in particular can be applied at varying dosages. Application should be effected in such a way that a disease is combated as effectively as possible or the onset of such a disease is prevented by a prophylactic administration. Concentration and type of application can be determined by a person skilled in the art using routine tests. Preferred applications of the compounds of the invention are oral application in the form of powders, tablets, fluid mixture, drops, capsules or the like, rectal application in the form of suppositories, solutions and the like, parenteral application in the form of injections, infusions and solutions, and local application in the form of ointments, pads, dressings, lavages and the like. Contacting with the compounds according to the invention is preferably effected in a prophylactic or therapeutic fashion.

For example, the suitability of the selected form of application, of the dose, application regimen, selection of adjuvant and the like can be determined by taking serum aliquots from the patient, i.e., human or animal, and testing for the presence of indicators of disease in the course of the treatment procedure. Alternatively or concomitantly, the condition of the kidneys, but also, the amount of T cells or other cells of the immune system can be determined in a conventional manner so as to obtain a general survey on the immunologic constitution of the patient and, in particular, the constitution of organs important to the metabolism. Additionally, the clinical condition of the patient can be observed for the desired effect. Where insufficient therapeutic effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration.

In addition to oral ingestion, e.g. intramuscular or subcutaneous injections or injections into the blood vessels can be envisaged as another preferred route of therapeutic administration of the compounds according to the invention. At the same time, supply via catheters or surgical tubes can also be used, e.g. via catheters directly leading to particular organs such as the kidneys.

In a preferred embodiment the compounds according to the invention can be employed in a total amount of 0.05 to 500 mg/kg body weight per 24 hours, preferably 5 to 100 mg/kg body weight. Advantageously, this is a therapeutic quantity which is used to prevent or improve the symptoms of a disorder or of a responsive, pathologically physiological condition.

Obviously, the dose will depend on the age, health and weight of the recipient, degree of the disease, type of required simultaneous treatment, frequency of the treatment and type of the desired effects and side-effects. The daily dose of 0.05 to 500 mg/kg body weight can be applied as a single dose or multiple doses in order to furnish the desired results. In particular, pharmaceutical agents are typically used in about 1 to 10 administrations per day, or alternatively or additionally as a continuous infusion. Such administrations can be applied as a chronic or acute therapy. It will be appreciated that the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 10 times per day with the compounds of the invention or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosage required in each case and the type of application of the active substances.

In another particularly preferred embodiment of the invention the pharmaceutical agent is used in a single administration of from 1 to 100, especially from 2 to 50 mg/kg body weight. In the same way as the total amount per day, the amount of a single dose per application can be varied by a person of specialized knowledge in the art. Similarly, the compounds used according to the invention can be employed in veterinary medicine with the above-mentioned single concentrations and formulations together with the feed or feed formulations or drinking water. A single dose preferably includes that amount of active substance which is administered in one application and which normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to the invention is distributed over 2 to 10 applications, preferably 2 to 7, and more preferably 3 to 5 applications. Of course, continuous infusion of the agents according to the invention is also possible.

In a particularly preferred embodiment of the invention, 1 to 2 tablets are administered in each oral application of the compounds of the invention. The tablets according to the invention can be provided with coatings and envelopes well-known to those skilled in the art or can be composed in a way so as to release the active substance(s) only in preferred, particular regions of the host.

It is preferred in another embodiment of the invention that the compounds according to the invention are optionally associated with each other or, coupled to a carrier, enclosed in liposomes, and, in the meaning of the invention, such enclosure in liposomes does not necessarily imply that the compounds of the invention are present inside the liposomes. Enclosure in the meaning of the invention may also imply that the compounds of the invention are associated with the membrane of the liposomes, e.g. in such a way that the compounds are anchored on the exterior membrane. Such a representation of the inventive compounds in or on liposomes is advantageous in those cases where a person skilled in the art selects the liposomes such that the latter have an immune-stimulating effect. Various ways of modifying the immune-stimulating effect of liposomes are known to those skilled in the art from DE 198 51 282. The lipids can be ordinary lipids, such as esters and amides, or complex lipids, e.g. glycolipids such as cerebrosides or gangliosides, sphingolipids or phospholipids.

For example, it is possible to replace single amino acids or groups of amino acids without adversely affecting the activity of the peptides with respect to accomplishing the object of the present invention. For replacement of such amino acids, reference is made to appropriate standard textbooks of biochemistry and genetics.

Various ways of preparing peptides have been disclosed in the prior art. Peptides designed starting from the peptides of the invention using such methods are included in the teaching according to the invention. For example, one way of generating functionally analogous peptides has been described in PNAS USA 1998, Oct. 13, 9521, 12179-84; WO 99/6293 and/or WO 02/38592, and the above teachings are hereby incorporated in the disclosure of the invention. That is, all peptides, peptide fragments or structures comprising peptides generated using the methods mentioned above—starting from the peptides of the invention—are peptides in the meaning of the invention, provided they accomplish the object of the invention. Furthermore, the peptides according to the invention are lead structures for the development of peptide mimetics.

As is well-known to those skilled in the art, some amino acids have analogous physicochemical properties so that these amino acids advantageously can be replaced by each other. For example, these include the group of amino acids (a) glycine, alanine, valine, leucine and/or isoleucine; or the amino acids (b) serine and threonine, the amino acids (c) asparagine and glutamine, the amino acids (d) aspartic acid and glutamic acid; the amino acids (e) lysine and arginine, as well as the group of aromatic amino acids (f) phenylalanine, tyrosine and/or tryptophan. Amino acids within one and the same group (a-f) can be replaced with one another. Furthermore, the amino acids can be replaced by modified amino acids or specific enantiomers. Further modifications are possible in accordance with the teaching of WO 99/62933 or WO 02/38592 which hereby are incorporated in the disclosure of the teaching of the invention.

In another preferred embodiment the peptide comprises a linker and/or a spacer selected from the group comprising α-aminocarboxylic acids as well as homo- and heterooligomers thereof, α,ω-aminocarboxylic acids and branched homo- or heterooligomers thereof, other amino acids, as well as linear and branched homo- or heterooligomers (peptides); amino-oligoalkoxyalkylamines; maleinimidocarboxylic acid derivatives; oligomers of alkylamines; 4-alkylphenyl derivatives; 4-oligoalkoxyphenyl or 4-oligoalkoxyphenoxy derivatives; 4-oligoalkylmercaptophenyl or 4-oligoalkylmercaptophenoxy derivatives; 4-oligoalkylaminophenyl or 4-oligoalkylaminophenoxy derivatives; (oligoalkylbenzyl)phenyl or 4-(oligoalkylbenzyl)phenoxy derivatives, as well as 4-(oligoalkoxybenzyl)phenyl or 4-(oligoalkoxybenzyl)phenoxy derivatives; trityl derivatives; benzyloxyaryl or benzyloxyalkyl derivatives; xanthen-3-yloxyalkyl derivatives; (4-alkylphenyl)- or ω-(4-alkylphenoxy)alkanoic acid derivatives; oligoalkylphenoxyalkyl or oligoalkoxyphenoxyalkyl derivatives; carbamate derivatives; amines; trialkylsilyl or dialkylalkoxysilyl derivatives; alkyl or aryl derivatives and/or combinations thereof; other possible structures have been described in EP 1 214 350 which hereby is incorporated in the disclosure of the invention.

In a preferred fashion, synthetic peptides or fragments thereof can be multimerized by chemical crosslinkers or coupled to a carrier molecule such as BSA, dextran, KLH or others. Chemical crosslinkers used to this end are listed in “Bioconjugate Techniques”, Greg T. Hermanson, Academic Press, 1996, which hereby is incorporated in the disclosure of the teaching according to the invention. Preferred crosslinkers are homobifunctional crosslinkers, preferably NHS esters such as DSP, DTSSP, DSS, BS, DST, sulfo-DST, BSOCOES, sulfo-BSOCOES, EGS, sulfo-EGS, DSG or DSC, homobifunctional imidoesters such as DMA, DMP, DMS or DTBP, homobifunctional sulfhydryl-reactive crosslinkers such as DPDPB, BMH or BMOE, difluorobenzene derivatives such as DFDNB or DFDNPS, homobifunctional photoreactive crosslinkers such as BASED, homobifunctional aldehydes such as formaldehyde or glutaraldehyde, bisepoxides such as 1,4-butanediol diglycidyl ethers, homobifunctional hydrazides such as adipic dihydrazides or carbohydrazides, bisdiazonium derivatives such as bis-diazotized o-tolidine, benzidine or bisalkylhaloid.

Also preferred are heterobifunctional crosslinkers, especially amine-reactive and sulfhydryl-reactive crosslinkers such as SPDP, LC-SPDP, sulfo-LC-SPDP, SMPT, sulfo-LC-SMPT, SMCC, sulfo-SMCC, MBS, sulfo-MBS, SIAB, sulfo-SIAB, SMPB, sulfo-SMBP, GMBS, sulfo-GMBS, SIAX, SIAXX, SIAC, SIACX or NPIA, carbonyl-reactive and sulfhydryl-reactive crosslinkers such as MPBH, M₂C₂H or PDPH, amine-reactive and photoreactive crosslinkers such as NHS-ASA, sulfo-NHS-ASA, sulfo-NHS-LC-ASA, SASD, HSAB, sulfo-HSAB, SANPAH, sulfo-SANPAH, ANB-NOS, SAND, SADP, sulfo-SADP, sulfo-SAPB, SAED, sulfo-SAMCA, p-nitrophenyldiazopyruvate or PNP-DTP, sulfhydryl- and photoreactive crosslinkers such as ASIB, APDP, benzophenone-4-iodoacetamide or benzophenone-4-maleinimide, carbonyl-reactive and photoreactive crosslinkers such as ABH, carboxylate-reactive and photoreactive crosslinkers such as ASBA, arginine-reactive crosslinkers such as APG, trifunctional crosslinkers such as 4-azido-2-nitrophenylbiocytin 4-nitrophenyl ester, sulfo-SEBD, TSAT and/or TMEA.

In another preferred embodiment of the invention the peptides of the invention and structures produced in a recombinant fashion are linked by peptide bridges having a length of from 0 to 50 amino acids. Also included are recombinant proteins consisting of two N-terminal and one C-terminal sequence, or hexamers consisting of three N-terminal sequences and three C-terminal sequences, or multimers of the above-mentioned recombinant structures, wherein a peptide bridge of 0 to 50 amino acids can be pre-sent between each of the N- and C-terminal sequences. For purification, solubilization, or changes in conformation, the peptides can be provided with specific fusion components either on the N or C terminus, such as CBP (calmodulin binding protein), His-tag and/or others. Similar constructs can also be encoded by DNA used in therapy.

The invention also relates to a kit comprising a nucleic acid molecule of the invention, a vector of the invention, a host cell of the invention, a polypeptide of the invention, a recognition molecule of the invention and/or a pharmaceutical composition, optionally together with information—e.g. an instruction leaflet or an internet address referring to homepages including further information, etc.—concerning handling or combining the contents of the kit. For example, the information concerning handling the contents of the kit may comprise a therapeutic regimen for edemas, cardiac failure, liver cirrhosis, hyperinsulinism, hypertony, duodenal ulcer. Also, the information may comprise explanations referring to the use of the materials and products of the invention in diagnosing diseases associated with AKAP-PKA interaction or decoupling thereof. The kit according to the invention may also be used in basic research. In basic research, the kit can preferably be used to detect whether a metabolic phenomenon is associated with interaction or absent interaction of AKAP and PKA. More specifically, the kit according to the invention allows to determine which subunits of AKAP and/or PKA are responsible for interaction of the above two molecules or failure of such interaction to take place.

The products of the invention, such as peptides, vectors, nucleic acid molecules, may comprise other advantageous nucleic acids, amino acids, carbohydrates or lipids. For example, it may be preferred to modify the peptides with a fatty residue, such as stearate, in such a way that the peptides have good membrane permeability. These peptides can be used to perform experiments on cell cultures. Such peptides can be used as tools to effect particularly efficient decoupling of PKA from AKAP proteins in cells, cell cultures, tissue cultures, organ cultures or organisms. More specifically, the peptides in the meaning of the invention can be used in cell cultures to answer the question whether a particular process depends on anchoring of the PKA on AKAP proteins. Owing to the advantageous high affinity for human RIIα subunits of PKA, the peptides according to the invention are suitable especially for investigations in human systems. By comparison with peptides binding PKA with different affinity it will also be possible to make quantitative statements defining to what extent PKA-AKAP interaction is necessary to ensure the progress of a physiological process. In particular, the kits according to the invention can be used to study the progress of such a physiological process. Advantageously, the peptides according to the invention bind the RII subunits of PKA more strongly than the typical PKA binding domains of AKAP18δ.

Advantageously, the peptides of the invention have RIIα or RIIβ specificity so that the kit can be used e.g. to obtain highly detailed insight into the interaction. More specifically, decoupling of one or another regulatory subunit of PKA from AKAP proteins may furnish information as to which PKA, type IIα or type IIβ, is involved in the respective process to be investigated. In particular, the peptide A18δRIIβRnl selectively binds RIIβ subunits of PKA.

The invention also relates to a method for the modification, especially inhibition, and preferably decoupling, of an AKAP-PKA interaction or an interaction of AKAP or PKA subunits, comprising the steps of:
a) providing a nucleic acid molecule of the invention, a vector of the invention, a host cell of the invention and/or a polypeptide of the invention, and
b) contacting at least one product according to a) with a cell, a cell culture, a tissue and/or a target organism.

In a preferred fashion the interaction is analyzed or modified on a regulatory R subunit and more preferably on an RIIα and/or RIIβ subunit.

The invention also relates to the use of a nucleic acid molecule of the invention, a host cell of the invention, an organism of the invention, a polypeptide of the invention, a recognition molecule of the invention, a pharmaceutical composition of the invention and/or a kit of the invention for the modification, especially inhibition, of an AKAP-PKA interaction. The invention also relates to the use of fragments or partial regions of the peptides or nucleic acids according to the invention. Furthermore, extension of the peptides or nucleic acids of the invention by additional amino acids or nucleotides can be envisaged. Of course, it is also possible to modify the peptides with lipid or carbohydrate structures.

In a preferred embodiment of the invention, especially of the use according to the invention, the cell—e.g. as a cell culture—or the organism is used as a model for tissue—and/or cell-specific AKAP-PKA interaction, particularly as a model for insipid diabetes. Other preferred models are cell cultures or tissues comprising the nucleic acid molecules or peptides of the invention.

In another preferred embodiment of the invention the vasopressin-induced redistribution of AQP2 is modified, particularly prevented, as a result of the AKAP-PKA modification.

In another particularly preferred embodiment the polypeptide and/or the pharmaceutical composition are used as agents causing loss of water, particularly as aquaretic agents.

In another preferred embodiment of the invention, especially of the use according to the invention, the interaction of the RIIα or RIIβ subunit of PKA with AKAP is modified, particularly inhibited.

In another preferred use, the subunits are of human or murine origin.

Without intending to be limiting, the invention will be explained in more detail with reference to the following examples.

### Peptides for the Inhibition of the Interaction of Protein Kinase A and Protein Kinase A Anchor Proteins

### Materials and Methods

### Preparation—on Membranes—of Peptide Libraries Derived From the Sequence of the PKA Binding Domain of AKAP18δ

All chemicals and solvents were purchased from Fluka (Steinheim) or Sigma Aldrich (Munich) and used without further purification steps. Fmoc-protected amino acid penta-fluorophenyl esters were purchased from Novabiochem Merck Biosciences GmbH (Darmstadt).

Peptide libraries were synthesized by means of automatic SPOT synthesis on Whatman 50 cellulose membranes according to standard protocols using Fmoc chemistry and AutoSpot Robot ATE 222 (Intavis Bioanalytical Instruments AG, Cologne). The protective groups of the amino acid side chains were removed using a mixture of trifluoroacetic acid (TFA) in dichloromethane (DCM) (Frank, 1992; Kramer and Schneider-Mergener, 1998). For control, spots (about 50 nmol of peptide per spot) were cut from the cellulose membrane, removed from the membrane by treatment with 0.05 M NaOH, and analyzed using HPLC and MALDI-TOF mass spectrometry.

### Detection of Membrane-Associated Peptides in an RII Overlay Experiment, Using Regulatory RIIα and RIIβ Subunits of PKA as Probe

### Materials

1. Regulatory RIIα (human) and RIIβ (rat) subunits of PKA, obtained from Prof. Dr. Friedrich W. Herberg, University of Kassel, Germany.
2. Catalytic subunits of PKA, Promega, Mannheim, Germany, Order No. V5161
3. [γ-³²P]ATP, 5000 Ci/mmol, Amersham Biosciences, Brunswick, Germany, Order No. AA0018
4. Sephadex G 50, medium Pharmacia, Order No. 17-0043-01
5. Phosphate-buffered saline (PBS)

| | |
|---|---|
| NaCl | 8 g |
| KCl | 0.2 g |
| Na₂HPO₄ | 1.44 g |
| KH₂PO₄ | 0.24 g |

are dissolved in 800 ml H₂O, adjusted to pH 7.4 and filled up with H₂O to make 1 liter
6. Tris-buffered saline with Tween 20

| Tris-HCl | 10 mM |
|---|---|
| NaCl | 150 mM |
| Tween | 200.05% |
| pH | 7.5 |

### Radioactive Labelling of the Regulatory Subunit of PKA

1. Reaction batch

| | | | Stock | | | |
|---|---|---|---|---|---|---|
| | Final concentration | | soln. | | in batch | |
| RIIα or RIIβ | 15 | μg | 2.7 | μg/μl | 5.6 | μl |
| Catalytic subunit | 2 | μg | 0.9 | μg/μl | 2 | μl |
| of PKA | | | | | | |
| Potassium phosphate | 25 | mM | 1 | M | 12.5 | μl |
| buffer, pH 7.0 | | | | | | |
| cAMP | 10 | μM | 1 | mM | 5 | μl |
| MgCl₂ | 10 | mM | 0.5 | M | 10 | μl |
| DTT | 0.5 | mM | 50 | mM | 5 | μl |
| [γ-³²P]ATP/ATP | 0.1 | μM | | | | |

| | | | | |
|---|---|---|---|---|
| radioactive: 3.3 × 10⁸ cpm/ml = 75 μCi | 5 | μCi/μl | 15 | μl |
| non-radioactive: | 10 | μM | 5 | μl |
| H₂O | 434.9 | μl | | |
| 10 min incubation at 0° C. (on ice) | | | | |

### 2. Adjusting the ATP Concentration

The ATP concentration was adjusted to 10 μM by addition of non-radioactive ATP (addition of 5 μl of a 1 mM solution). The batch was incubated on ice for another 50 min.

### 3. Quenching and Checking the Reaction

The reaction was quenched by adding dextran blue and removing free nucleotides. The free ATP was removed on a Sephadex G50 column.

Separation of Labelled RII Subunit of PKA from Free Nucleotides on Sephadex G50 Columns

Non-incorporated nucleotides were separated from the RII subunits by fractionation on Sephadex G 50 columns.
1. Swelling of the Sephadex G 50 material: 20 g thereof was allowed to swell in 400 ml of PBS at room temperature overnight. Non-settled material was subsequently removed with a Pasteur pipette. The swollen material was aliquoted in 50 ml Falcon tubes and stored at 4° C. For preservation, sodium azide was added to make a final concentration of 0.01%.
2. The material was poured into a 10 ml sterile disposable pipette sealed with a glass sphere. To settle the column bed, 50 ml of PBS containing 1 mg/ml BSA (bovine serum albumin) was allowed to pass.

Until used, the column was sealed with parafilm at the top thereof.
3. The labelled RII subunits (500 μl), together with dextran blue (70 μl of a 20 mg/ml solution), were applied on the column (overall volume=570 μl).
4. The sample was allowed to migrate into the matrix, followed by filling up with PBS.
5. A short time before the dextran blue was eluted, collection of fractions was begun (2 fractions of 1.5 ml each, the other fractions 1 ml each).
6. To determine the incorporation of ³²P, 1% (5.7 μl) of sample upstream of the column (corresponding to 1% of the radioactivity employed) and 3 μl of each fraction were used.
7. The fractions of the first peak including the probe were combined. The incorporation rate in % was calculated and the specific activity (cpm/μg of protein) was determined.

### RII Overlay

1. The proteins (40 μg) were separated by means of SDS-PAGE and transferred on a PVDF membrane (PVDF: polyvinylidene fluoride) using a semi-dry electroblotting procedure. The membrane-associated proteins were stained with Ponceau S in order to identify the marker proteins on the membrane. Destaining was effected using TBS.
2. The membrane was incubated in Blotto/BSA at 4° C. for 16 hours:

| 10 mM potassium phosphate buffer | pH 7.4 | |
|---|---|---|
| 0.15 M NaCl | 8.766 | g/l |
| 5% (w/v) skimmed milk powder | 50 | g/l |
| 0.1% (w/v) BSA | 1 | g/l |
| (0.01% antifoam (Sigma)) | | |
| 0.02% NaN₃ | 0.2 | g/l |

3. Blotto/BSA was replaced with fresh one and ³²P-labelled RII subunits were added (10⁵ cpm/ml). This was incubated for 4-6 h at room temperature.
4. The membrane was washed for 4×15 min in Blotto/BSA and for 2×10 min in 10 mM potassium phosphate buffer, pH 7.4, 0.15 M NaCl.
5. RII-binding proteins were detected by exposition on a phosphoimager plate.

### Results

A peptide library derived from the wild-type amino acid sequence of the PKA binding domain of AKAP18δ (PEDAELVRLSKRLVENAVLKAVQQY; Henn et al., 2004) was synthesized on a membrane. To this end, each amino acid of the wild-type sequence was substituted with the 20 possible amino acids. FIG. 1 shows the detection of the peptides by means of the RII overlay method. In this case, radioactive PKA RIIα and RIIβ subunits were used simultaneously as probe. Either RIIα or RIIβ subunits were used as probe in all subsequent experiments. The result shows marked differences in the ability of binding of the single peptides to the R subunits (varying signal intensities).

FIG. 2 shows a repetition of the experiment using selected peptides (AKAP18δ-L304T, AKAP18δ-L308D, AKAP18δ-L314E), wherein, however, their ability of binding to RIIα or RIIβ subunits was tested separately in various RII over-lay experiments. As controls, the peptides Ht31, Ht31-P, AKAP18δ-RI and AKAP18δ-wt (wild-type sequence) were synthesized on the same membranes and subjected to the RII over-lay experiment. For quantification, the signals were evaluated by means of densitometry and correlated with the signal obtained for AKAP18δ-wt. The quantification suggests stronger binding of AKAP18δ-L304T and AKAP18δ-L314E to RIIα as well as RIIβ subunits compared to that of AKAP18δ-RI and AKAP18δ-L308D, which is weaker. The well-known peptide Ht31 binds the two regulatory subunits about 5 times weaker than AKAP18δ-wt and about 5-6 times weaker than AKAP18δ-L304T and AKAP18δ-L314E. Binding of Ht31 to the regulatory RIIα and RIIβ subunits used herein is only slightly stronger than binding of the subunits to Ht31-P which does not inhibit the AKAP-PKA interaction (Klussmann et al., 1999; Alto et al., 2003). Consequently, the peptides AKAP18δ-wt, AKAP186-L304T and AKAP18δ-L314E are substantially more efficient inhibitors of an AKAP-PKA interaction compared to Ht31.

Alto et al. (2003) have developed a peptide, AKAP_{IS}, which inhibits the interaction between the murine RIIα subunit of PKA with an affinity increased by 5 times (K_{D}=0.45 nM) compared to the Ht31 peptide (K_{D}=2.2 nM).

In our RII overlay experiments the peptides AKAP_{IS} and Ht31 barely bind the human RIIα and the RIIβ subunit of PKA from rats; in contrast, the peptides AKAP18δ-wt, AKAP18δ-L304T and AKAP18δ-L314E identified by us bind strongly. This result suggests species-related differences between the murine and human RIIα subunits, resulting in different binding affinities for the same peptides.

### Identification of Peptides Specifically Binding RIIβ Sub-Units of PKA

To find peptides binding either RIIα or RIIβ subunits of PKA, thus specifically inhibiting the interaction of AKAP proteins with the type IIα or type IIβ PKA, peptides that might block the AKAP binding pocket were derived by means of three-dimensional structural models of the PKA subunits from the wild-type PKA binding domain of AKAP18δ. The peptides (1-19) were synthesized in parallel on two membranes and subsequently tested in RII overlay experiments for their binding ability to RIIα or RIIβ subunits of PKA (FIG. 3A). The quantitative evaluation showed, inter alia, a marked difference in binding of the two PKA subunits to peptide No. 7, the sequence of which, along with those of other peptides, is listed in FIG. 3B.

Starting from the sequence of peptide 7, two peptide libraries were synthesized on membranes and subjected to RII overlay experiments using RIIα and RIIβ subunits, respectively, as probes. FIG. 4 shows that some peptides bind RIIα, but no RIIβ subunits (for example, the peptides 10/11 and 10/12) and vice versa (for example, peptide 21/4). Moreover, some peptides have stronger binding to RIIα sub-units as compared to RIIβ subunits, while the reverse applies to others which give weaker binding of RIIα subunits compared to RIIβ subunits. In summary, the results show that we found the first blockers in the above-mentioned peptides A18δRIIα Hs1 and 2 and A18δRIIβRnl, which selectively identify the interaction of RIIα or RIIβ subunits of PKA with AKAP proteins.

TABLE 1

| | | |
|---|---|---|
| No. A | ANDAQLVRLSKRLVENAVLKAVQQY | |
| No. B | ANDAQLVRLSKRLVENAVLKAVQQY | |
| No. C | ASDAQLVRLSKRLVENAVLKAVQQY | |
| No. D | ASDAKLVRLSKRLVENAVLKAVQQY | |
| No. E | ARDAKLVRLSKRLVENAVLKAVQQY | |
| No. F | ARDAQLVRLSKRLVENAVLKAVQQY | |
| No. G | ANDARLVRLSKRLVENAVLKAVQQY | |
| No. H | ASDARLVRLSKRLVENAVLKAVQQY | |
| No. I | ASDAKTVRLSKRLVENAVLKAVQQY | |
| No. J | ANDAKTERLSKRLVENAVLKAVQQY | |
| No. K | ANDAKTERLSQRLVENAVLKAVQQY | |
| No. L | ANDAKTQRLSQRLVENAVLKAVQQY | |
| No. M | PEDAELVRLSKRLVENAVLKAVQQY | |
| No. N | PEDAELVRLSKRLVENAVLQAVQQY | |
| No. O | PEDAELVRLSKRLVENAVLNGVQQY | |
| No. P | PEDAELVRLSKRLVENAVLNGQQQY | |
| No. Q | PEDAELVRLSKRLVENAVLNGNQQY | |
| No. R | PEDAELVRLSKRLVENAVKNGNQQY | |
| No. S | PEDAELVRLSKRLVENAVKNGAQDY | |
| No. T | PEDAELVRLSKRLVENAVLKAVQQY | (Akap18δ-wt) |
| No. U | PEDAELVRTSKRLVENAVLKAVQQY | (AKAP18δ-L304T) |
| No. V | PEDAELVRLSKRDVENAVLKAVQQY | (AKAP18δ-L308D) |
| No. W | PEDAELVRLSKRLVENAVEKAVQQY | (AKAP18δ-L314E) |
| No. X | PEDAELVRLSKRLPENAVLKAVQQY | (AKAP18δ-P) |
| No. Y | PEDAELVRLSKRLPENAPLKAVQQY | (AKAP18δ-P) |
| No. Z | PEDAELVRLDKRLPENAPLKAVQQY | (AKAP18δ-phos) |
| No. AA | EPEDAELVRLSKRLVENAVLKAVQQYLEETQ | (Akap18δ-RI) |
| No. BB | ANDARLVRLSKRLRENAVLKAVQQY | (A18δRIIαHs1 (14/14)) |
| No. CC | ANDARLVRLSKRLYENAVLKAVQQY | (A18δRIIαHs2 (14/19)) |
| No. DD | ANDARLVRLSKRLVENAVLKFVQQY | (A18δRIIβRn1 (21/4)) |
| No. EE | ANDARLVRLNKRLVENAVLKAVQQY | (A18δRIIαRn2 (10/11)) |
| No. FF | ANDARLVRLPKRLVENAVLKAVQQY | (A18δRIIαRn3 (10/12)) |
| No. GG | ANDARLVRLSKRDVENAVLKAVQQY | (A18δRIIαRn4 (13/02)) |
| No. HH | YQEQLEEEVAKVIVSMSIAFAQQTE | (AKAP450_1) |
| No. II | NLQKIVEEKVAAALVSQIQLEAVQE | (AKAP450_2) |
| SEQ ID No. 1 | PEDAELVRLSKRLVENAVLKAVQQY | (AKAP18δ-wt) |
| SEQ ID No. 2 | PEDAELVRTSKRLVENAVLKAVQQY | (AKAP18δ-L304T) |
| SEQ ID No. 3 | PEDAELVRLSKRDVENAVLKAVQQY | (AKAP18δ-L308D) |
| SEQ ID No. 4 | PEDAELVRLSKRLVENAVEKAVQQY | (AKAP18δ-L314E) |
| SEQ ID No. 5 | PEDAELVRLSKRLPENAVLKAVQQY | (AKAP18δ-P) |
| SEQ ID No. 6 | PEDAELVRLSKRLPENAPLKAVQQY | (AKAP18δ-PP) |
| SEQ ID No. 7 | PEDAELVRLDKRLPENAPLKAVQQY | (AKAP18δ-phos) |
| SEQ ID No. 8 | EPEDAELVRLSKRLVENAVLKAVQQYLEETQ | (Akap18δ-RI) |
| SEQ ID No. 9 | NTDEAQEELAWKIAKMIVSDIMQQA | |
| SEQ ID No. 10 | VNLDKKAVLAEKIVAEAIEKAEREL | |
| SEQ ID No. 11 | NGILELETKSSKLVQNIIQTAVDQF | |
| SEQ ID No. 12 | TQDKNYEDELTQVALALVEDVINYA | |
| SEQ ID No. 13 | LVDDPLEYQAGLLVQNAIQQAIAEQ | |
| SEQ ID No. 14 | QYETLLIETASSLVKNAIQLSIEQL | |
| SEQ ID No. 15 | LEKQYQEQLEEEVAKVIVSMSIAFA | |
| SEQ ID No. 16 | EEGLDRNEEIKRAAFQIISQVISEA | |
| SEQ ID No. 17 | ETSAKDNINIEEAARFLVEKILVNH | |
| SEQ ID No. 18 | ADRGSPALSSEALVRVLVLDANDNS | |
| SEQ ID No. 19 | SDRGSPALSSEALVRVLVLDANDNS | |
| SEQ ID No. 20 | TDRGFPALSSEALVRVLVLDANDNS | |
| SEQ ID No. 21 | FLAGETESLADIVLWGALYPLLQDP | |
| SEQ ID No. 22 | SELLKQVSAAASVVSQALHDLLQHV | |
| SEQ ID No. 23 | EKESLTEEEATEFLKQILNGVYYLH | |
| SEQ ID No. 24 | EKGYYSERDAADAVKQILEAVAYLH | |
| SEQ ID No. 25 | WLYLQDQNKAADAVGEILLSLSYLP | |
| SEQ ID No. 26 | LKISPVAPDADAVAAQILSLLPLKF | |
| SEQ ID No. 27 | SKTEQPAALALDLVNKLVYWVDLYL | |
| SEQ ID No. 28 | VLASAYTGRLSMAAADIVNFLTVGS | |
| SEQ ID No. 29 | VKLSNLSNLSHDLVQEAIDHAQDLQ | |
| SEQ ID No. 30 | APSDPDAVSAEEALKYLLHLVDVNE | |
| SEQ ID No. 31 | QMKAKRTKEAVEVLKKALDAISHSD | |
| SEQ ID No. 32 | KDKLKPGAAEDDLVLEVVIMIGTVS | |
| SEQ ID No. 33 | EKRVADPTLEKYVLSVVLDTINAFF | |
| SEQ ID No. 34 | QENLSLIGVANVFLESLFYDVKLQY | |
| SEQ ID No. 35 | HQSVVYRKQAAMILNELVTGAAGLE | |
| SEQ ID No. 36 | QQLQKQLKEAEQILATAVYQAKEKL | |
| SEQ ID No. 37 | HSVMDTLAVALRVAEEAIEEAISKA | |
| SEQ ID No. 38 | RQVQETLNLEPDVAQHLLAHSHWGA | |
| SEQ ID No. 39 | DIPSADRHKSKLIAGKIIPAIATTT. | |

TABLE 2
Binding constants for the interaction of human
RIIα and RIIβ subunits from rats with the specified peptides
derived from the wild-type RII binding domain of
AKAP18δ (AKAP18δ-wt).

| | RIIα binding | RIIβ binding |
|---|---|---|
| Peptide | K_{D} [nM] | K_{D} [nM] |
| AKAP18δ-wt | 0.4-1.5 | 1-6 |
| AKAP18δ-304T | 0.3-0.9 | 35 |
| AKAP18δ-L314E | 0.2-1.3 | 22 |
| AKAP18δ-L308D | no binding | no binding |
| AKAP18δ-P | no binding | no binding |
| AKAP18δ-PP | no binding | no binding |
| Ht3 | 15 | 35 |
| Ht31-P | no binding | no binding |
| AKAP_{IS} | no binding | no binding |
| AKAP_{IS}-P | no binding | no binding |

The values were obtained by means of surface plasmon resonance measurements.
L, leucine,
T, threonine,
D, aspartate,
P, proline,
IS, in silico.
304, 308 and 314 denote the position of the corresponding amino acids in AKAP18δ.

### Further Peptides Inhibiting AKAP-PKA Interactions

In addition to the peptides described in FIGS. 1-4, it was possible to identify others acting as inhibitors of AKAP-PKA interactions. To detect this property, the peptides listed below were synthesized on a cellulose membrane (SPOT synthesis method) and subjected to an RII overlay (FIG. 5). All black dots represent peptides having bound the regulatory PKA subunits. The peptides were synthesized in six blocks. The peptides of column A, positions 1-17, are positive controls and identical in all blocks. The names of the peptides listed below are derived from their coordinates in blocks 1-6, e.g. the peptide 1.B13 (sequence: YIALNEDLRSWTAADTAAQISQRKL) can be found in block 1, column B, at position 13.

FIG. 5 shows the identification of peptides inhibiting the AKAP-PKA interactions. Candidate peptides were synthesized on a membrane and incubated with radiolabelled regulatory RIID subunits of PKA (RII overlay experiment). All black dots represent peptides having bound regulatory PKA sub-units (detected using a phosphoimager). The peptide sequences are presented in the attached list (Table 3):

TABLE 3
Peptide sequences

| | | |
|---|---|---|
| No | Sequence | Name/ID-Nr. |

| | | |
|---|---|---|
| 1 | YIALNEDLRSWTAADTAAQISQRKL | 1C17_HUMAN |
| 2 | ISKEHWNPTIVALVYNVLKTLMEMN | 2A5A_HUMAN |
| 3 | DAPEFHSRYITTVIQRIFYTVNRSW | 2ACA_HUMAN |
| 4 | GSTFQNTYNLKDIAGEAISFASGKI | 2ACA_HUMAN |
| 5 | LSRYNDQASSSRIIERIFSGAVTRG | 2ACA_HUMAN |
| 6 | EASEFHSRYITTVIQRIFYAVNRSW | 2ACC_HUMAN |
| 7 | QSEYSWRKMLSGIAAFLLGLIFLLV | 2DOB_HUMAN |
| 8 | LLLLPWLGWLGMLAGAVVIIVRAPR | 4F2_HUMAN |
| 9 | LYPHLAPKAEVGVIAKALVRLLRSH | A3B2_HUMAN |
| 10 | VKQNVKMSESQAALPSALKTLQQKL | A4E1_HUMAN |
| 11 | DPGALGRLGAWALLFFLVTTLLASA | AAAT_HUMAN |
| 12 | GQEVEGMNILGLVVFAIVFGVALRK | AAAT_HUMAN |
| 13 | EPTTGMDPKARRFLWNLILDLIKTG | ABC2_HUMAN |
| 14 | RLRGISWKDEARVVKWALEKLELTK | ABC2_HUMAN |
| 15 | LIQSLESNPLTKIAWRAAKPLLMGK | ABCR_HUMAN |
| 16 | AYTTQSLASVAYLINTLANNVLQML | ABI2_HUMAN |
| 17 | TLSKTKNLRLLILVGRLFMWEEPEI | ABM2_HUMAN |
| 18 | RYGAAEPHTIAAFLGGAAAQEVIKI | ABP1_HUMAN |
| 19 | NKDDDESPGLYGFLHVIVHSAKGFK | ABR_HUMAN |
| 20 | PTTHAMSPRLRHVLLELLPRLLGSP | ACHE_HUMAN |
| 21 | PGSAQEKSIERRFLNGLFSKLQPRD | AD13_HUMAN |
| 22 | HFDPTTAFRAPDVARALLRQTQVSR | AGRN_HUMAN |
| 23 | HYKETWKALEALVAKGLVQALGLSN | AKA1_HUMAN |
| 24 | QKLYLDRNLIAAVAPGAFLGLKALR | ALS_HUMAN |
| 25 | KEIKSYLKRIFQLVRFLFPELPEEG | ALS2_HUMAN |
| 26 | AISPWKTYQLVYFLDKILQKSPLPP | AMPB_HUMAN |
| 27 | GGAAGDEAREAAAVRALVARLLGPG | ANAG_HUMAN |
| 28 | GARSGHEQVVEMLLDRAAPTLSKTK | ANK3_HUMAN |
| 29 | SRTSSPVKSSLFLAPSALKLSTPSS | ANK3_HUMAN |
| 30 | TSYPWSWARVGPAVELALAQVKARP | ANPA_HUMAN |
| 31 | TIDRETSGNLEQLLLAVVKSIRSIP | ANX5_HUMAN |
| 32 | AVQNRFHGDAQVALLGLASVIKNTP | ANX9_HUMAN |
| 33 | PFRIYQTTTERPFIQKLFRPVAADG | APG5_HUMAN |
| 34 | RDRASYEARERHVAERLLMHLEEMQ | ARH1_HUMAN |
| 35 | EKLKSRPAHLGVFLRYIFSQADPSP | ARHB_HUMAN |
| 36 | NNTEKTVKKIKAFVEQVANVVLYSS | ARRS_HUMAN |
| 37 | EINVERDEKLIKVLDKLLLYLRIVH | ARS2_HUMAN |
| 38 | ELLQSETDKVVRAVAIALRNLSLDR | ARVC_HUMAN |
| 39 | LVASSQSVREAKAASHVLQTVWSYK | ARVC_HUMAN |
| 40 | EFKTLSEEEIEKVLKNIFNISLQRK | ARY1_HUMAN |
| 41 | EFKTLTEEEVEEVLKNIFKISLGRN | ARY2_HUMAN |
| 42 | VNILIGQTPISRLVALLVRGLGTEK | ASB8_HUMAN |
| 43 | GSAAMAASSVSVVLSSLFLKLYRKP | AT7A_HUMAN |
| 44 | PRSVRDTILSRALILKILMSAAIII | ATC4_HUMAN |
| 45 | EWTGYTAFFVGILVQQIADLIIRKT | ATHL_HUMAN |
| 46 | ADDQGQHPLLKMLLHLLAFSSAATG | ATIP_HUMAN |
| 47 | GLGGAWAFVLRDVIYTLIHYINQRP | ATM_HUMAN |
| 48 | STDEYYYYALAIVVMSIVSIVSSLY | ATY3_HUMAN |
| 49 | APWRKTTNPLDLAVMRLAALEQNVE | BA2A_HUMAN |
| 50 | MGALARALLLPLLAQWLLRAAPELA | BAE2_HUMAN |
| 51 | TTLLRLPQKVFDAVVEAVARASLIP | BAE2_HUMAN |
| 52 | DASYSLILEVQTAIDNVLIQSDVPI | BBS7_HUMAN |
| 53 | EKEEVPEGMEEAAVASVVLPARELQ | BC13_HUMAN |
| 54 | SHELRSKILSLQLLLSILQNAGPIF | BIG1_HUMAN |
| 55 | SHREAWTNLLLLFLTKVLKISDNRF | BIG1_HUMAN |
| 56 | HQSNKGYSLASLLAKVAAGKEKSSN | BIR6_HUMAN |
| 57 | LIQTSSTEQLRTIIRYLLDTLLSLL | BIR6_HUMAN |
| 58 | RGNLPTSGNISGFIRRLFLQLMLED | BIR6_HUMAN |
| 59 | VTFHLPHHVLKSIASAIVNELKKIN | BIR6_HUMAN |
| 60 | GAVYKGSLDERPVAVKVFSFANRQN | BMR2_HUMAN |
| 61 | LTKISLKNNLDHLLASLLFEEYISY | BP28_HUMAN |
| 62 | LVQLVDTLGAEKFLWILLILLFEQY | BP28_HUMAN |
| 63 | RLTSLKKTLATTLAPRVLLPAIKKT | BP28_HUMAN |
| 64 | PISGQTYSVEDAVLKGVVDPEFRIR | BPA1_HUMAN |
| 65 | WAKQHQQRLASALAGLIAKQELLEA | BPEA_HUMAN |
| 66 | HKAELKPLRADYLIARVVTVLEKLI | BPL1_HUMAN |
| 67 | ASTLILTPTSKDVLSNLVMISRGKE | BRC2_HUMAN |
| 68 | ENREKVNDQAKLIVGIVVGLLLAAL | C166_HUMAN |
| 69 | YRNGKVLHPLEGAVVIIFKKEMDPV | C166_HUMAN |
| 70 | NITDLGAYKLAEALPSLAASLLRLS | C2TA_HUMAN |
| 71 | DFRKKARQSIQGILEAAFSEELTRS | C3AR_HUMAN |
| 72 | SIGLQNFEIAKDFVVKVIDRLSRDE | CA16_HUMAN |
| 73 | VKPSSTRGGVLFAITDAFQKVIYLG | CA1E_HUMAN |
| 74 | RLGEQNFHKARRFVEQVARRLTLAR | CA26_HUMAN |
| 75 | SIGYTNFTLEKNFVINVVNRLDAIA | CA26_HUMAN |
| 76 | IYDERGARQLGLALGPALGLLGDPF | CA35_HUMAN |
| 77 | GSFAWHMNRSIVLLLKVLAQLRDHS | CABI_HUMAN |
| 78 | YKTSTVSADLANLLKRIATIVPRTE | CABI_HUMAN |
| 79 | HSGFIETEELKNFLKDLLEKANKTV | CABV_HUMAN |
| 80 | TNTQVEGDDEAAFLERLARREERRQ | CALD_HUMAN |
| 81 | FQLQRPPQNLLRLLRKAVERSSLMG | CANB_HUMAN |
| 82 | LYLRQNSMGLFSALRHALAKESLVG | CANC_HUMAN |
| 83 | DKVTQKQFQLKEIVLELVAQVLEHK | CAQ2_HUMAN |
| 84 | EGFHLLGQPLSHLARRLLDTVWNKG | CARF_HUMAN |
| 85 | ELYLRKRHHEPGVADRLIRLLQETS | CARF_HUMAN |
| 86 | LIRSLYEMQEERLARKAARGLNVGH | CARF_HUMAN |
| 87 | RLLDLATVKANGLAAFLLQHVQELP | CARF_HUMAN |
| 88 | EGFSFNPLKIEVFVQTLLHLAAKSF | CB80_HUMAN |
| 89 | RITQDAQLKSSKVVHKAVLQLNEEG | CBG_HUMAN |
| 90 | VKKAPDAEELDKVARLAAKALASVS | CBP4_HUMAN |
| 91 | MEQVAHQTIVMQFILELAKSLKVDP | CC37_HUMAN |
| 92 | FKITRYWNSLSNLVASLLNSVRSIA | CCAC_HUMAN |
| 93 | HIPTPGAALSWQAAIDAARQAKLMG | CCAC_HUMAN |
| 94 | NWLTEVQDTANKALLALFTAEMLLK | CCAN_HUMAN |
| 95 | PNSSKQTVLSWQAAIDAARQAKAAQ | CCAD_HUMAN |
| 96 | RTLLWTFIKSFQALPYVALLIAMIF | CCAF_HUMAN |
| 97 | VRHKYNFDNLGQALMSLFVLASKDG | CCAG_HUMAN |
| 98 | LRVISRAPGLKLVVETLISSLKPIG | CCAI_HUMAN |
| 99 | NYMFTTVFVLEAVLKLVAFGLRRFF | CCAI_HUMAN |
| 100 | VGNLGLLFMLLFFIYAALGVELFGK | CCAI_HUMAN |
| 101 | VHHKYNFDNLGQALMSLFVLASKDG | CCAI_HUMAN |
| 102 | FKITKYWTSLSNLVASLLNSIRSIA | CCAS_HUMAN |
| 103 | HNQFLWLTRLQDIANRVLLSLFTTE | CCAS_HUMAN |
| 104 | VKSKEEAQHVQRVLAQLLRREAALT | CD93_HUMAN |
| 105 | RVAQDLGLELAELVPRLFRVASKTH | CDA1_HUMAN |
| 106 | VFSRRGGLGARDLLLWLLLLAAWEV | CDA1_HUMAN |
| 107 | RIAQDLGLELEELVPRLFRVASKRH | CDA2_HUMAN |
| 108 | RRGRGAWTRLLSLLLLAAWEVGSGQ | CDA2_HUMAN |
| 109 | RIAQDLGLELAELVPRLFRVASKRH | CDAD_HUMAN |
| 110 | RIAQDLGLELAELVPRLFRVASKGR | CDA5_HUMAN |
| 111 | RIAQDLGLELAELVPRLFRMASKDR | CDA6_HUMAN |
| 112 | PTSNQQVKPLGLVLRKLLDREETPE | CDA9_HUMAN |
| 113 | RIAQDLGLELAELVQRLFRVASKRH | CDAA_HUMAN |
| 114 | ALLATQAGSAGGAVNKLVPRSVGAG | CDAB_HUMAN |
| 115 | RIAQDLGLELAELVQRLFRVASKTH | CDAB_HUMAN |
| 116 | VIIGPRGPGSQRLLLSLLLLAAWEV | CDAC_HUMAN |
| 117 | ADRGSPALSSEALVRVLVLDANDNS | CDB2_HUMAN |
| 118 | HYSVAEETESGSFVANLLKDLGLEI | CDB2_HUMAN |
| 119 | TDRGSPALSSEALVRVLVLDANDNS | CDBE_HUMAN |
| 120 | SDRGSPALSSEALVRVLVLDANDNS | CDB9_HUMAN |
| 121 | TDHGSPALSSEALVRVLVLDANDNS | CDBC_HUMAN |
| 122 | SDHGSPALSSEALVRVVVLDANDNS | CDBD_HUMAN |
| 123 | TDRGFPALSSEALVRVLVLDANDNS | CDBF_HUMAN |
| 124 | PFQRHPQRSEQVLLLTLLGTLWGAA | CDG6_HUMAN |
| 125 | TPRFLKEELEVKILENAAPSSRFPL | CDG6_HUMAN |
| 126 | RGPAGQRRMLFLFLLSLLDQVLSEP | CDGF_HUMAN |
| 127 | SGGGSDEGLASAAARGLVEKVRQLL | CDN5_HUMAN |
| 128 | TRMAAESRRVLLLAGRLAAQSLDTS | CENB_HUMAN |
| 129 | MGSEDHLGVIPRAIHDIFQKIKKFP | CENE_HUMAN |
| 130 | KRGPLLTALSAEAVASALHKLHQDL | CEP2_HUMAN |
| 131 | EYHYVQEKASKLAAASLLLALYMKK | CGB3_HUMAN |
| 132 | YIDENQDRYIKKLAKWVAIQSVSAW | CGL1_HUMAN |
| 133 | ANPVEIRRGVMLAVDAVIAELKKQS | CH60_HUMAN |
| 134 | PVAVRLQMSERNILSRLANRAPEPT | CHD4_HUMAN |
| 135 | ARERERLAHSRRAAARAVAAATAAV | CIK4_HUMAN |
| 136 | LKTISVIPGLKTIVGALIQSVKKLS | CIN9_HUMAN |
| 137 | LSRFEGMRVVVNALLGAIPSIMNVL | CIN4_HUMAN |
| 138 | LYILTPFNPLRKIAIKILVHSLFSM | CIN1_HUMAN |
| 139 | LYILTPFNPIRKLAIKILVHSLFNM | CIN2_HUMAN |
| 140 | LSRFEGMRVVVNALVGAIPSIMNVL | CIN5_HUMAN |
| 141 | LYILTPLNPVRKIAIKILVHSLFSM | CIN3_HUMAN |
| 142 | LKTITVIPGLKTIVGALIQSVKKLS | CIN4_HUMAN |
| 143 | LKTISVISGLKTIVGALIQSVKKLA | CIN5_HUMAN |
| 144 | LYVLSPFHPVRRAAVKILVHSLFNM | CIN5_HUMAN |
| 145 | LYILSPFNLIRRIAIKILIHSVFSM | CIN8_HUMAN |
| 146 | EEQLRLRERELTALKGALKEEVASR | CING_HUMAN |
| 147 | NYGKFEKGYLIFVVRFLPGLVNQER | CIS1_HUMAN |
| 148 | RGALLAGALAAYAAYLVLGALLVAR | CIW6_HUMAN |
| 149 | AQKVTRQEREEALVRGVFMKVVKPK | CJ11_HUMAN |
| 150 | MLRYDVYGGENEVIPEVLRKSHSHF | CJ24_HUMAN |
| 151 | ERQSAEVQGSLALVSRALEAAERAL | CK13_HUMAN |
| 152 | RSGYIEANELKGFLSDLLKKANRPY | CLB2_HUMAN |
| 153 | VRRKLGEDWIFLVLLGLLMALVSWS | CLC1_HUMAN |
| 154 | AYIVNYFMYVLWALLFAFLAVSLVK | CLC5_HUMAN |
| 155 | LIHSVSDAFSGWLLMLLIGLLSGSL | CLC5_HUMAN |
| 156 | YFPLKTLWRSFFAALVAAFTLRSIN | CLC5_HUMAN |
| 157 | RKKGRESYIETELIFALAKTSRVSE | CLH2_HUMAN |
| 158 | FAGSWRSGLAFLAVIKAIDPSLVDM | CLMN_HUMAN |
| 159 | GYFGSDVKVAYQLATRLLAHESTQR | CLR2_HUMAN |
| 160 | SPERFLSPLLGLFIQAVAATLATPP | CLR2_HUMAN |
| 161 | LQEQLYVRRAALAARSLLDVLPFDD | CLR3_HUMAN |
| 162 | WQERFLSPLLGRFLEGVAAVLATPA | CLR3_HUMAN |
| 163 | YFSQDVRVTARLLAHLLAFESHQQG | CLR3_HUMAN |
| 164 | LGSVIDISGLQRAVKEALSAVLPRV | CN2A_HUMAN |
| 165 | SFMEHIAMPIYKLLQDLFPKAAELY | CN2A_HUMAN |
| 166 | WVSFTSLGSLPSALRPLLSGLVGGA | CN3B_HUMAN |
| 167 | HVIYQRVDKAVGLAEAALGLARANN | CN93_HUMAN |
| 168 | TDMKDMRLEAEAVVNDVLFAVNNMF | CNC9_HUMAN |
| 169 | PKIVGRTKDVKEAVRKLAYQVLAEK | CND3_HUMAN |
| 170 | YGPTNFAPIINHVARFAAQAAHQGT | CNE1_HUMAN |
| 171 | LAHLRARLKELAALEAAAKHEELVE | CNG4_HUMAN |
| 172 | EEGGTPEQGVHRALQRLAHLLQADR | CNRC_HUMAN |
| 173 | WKGGSASTWLTAFALRVLGQVNKYV | CO5_HUMAN |
| 174 | DVLPNFFYHSNQVVRMAALEVYVRR | COA1_HUMAN |
| 175 | RQVQAEVPGSPIFVMRLAKQSRHLE | COA1_HUMAN |
| 176 | SSQFHMATNSSMFLKQAFEGEYPKL | COG5_HUMAN |
| 177 | VRRLERKYSSIPVIQGIVNEVRQSM | COG8_HUMAN |
| 178 | TDPDLPPGYVQSLIRRVVNNVNIVI | COH1_HUMAN |
| 179 | GDVKSKTEALKKVIIMILNGEKLPG | COPB_HUMAN |
| 180 | TFTLSTIKTLEEAVGNIVKRLGMHP | COPG_HUMAN |
| 181 | LLFGAWAGVLGTALSLLIRAELGQP | COX1_HUMAN |
| 182 | RFIFNRVVLEMEAVRAAAVSALAKF | CPG2_HUMAN |
| 183 | RREEATRQGELPLVKEVLLVALGSR | CPSA_HUMAN |
| 184 | NATLFTAAEIAPFVEILLTNLFKAL | CSE1_HUMAN |
| 185 | SVNWKHKDAAIVYLVTSLASKAQTQK | CSE1_HUMAN |
| 186 | QRREGGGRNIGGIVGGIVNFISEAA | CSS2_HUMAN |
| 187 | IEKESQRKSIDPALSMLIKSIKTKT | CT06_HUMAN |
| 188 | LDVIYWFRQIIAVVLGVIWGVLPLR | CT24_HUMAN |
| 189 | HRLLSTEWGLPSIVKSLIGAARTKT | CT45_HUMAN |
| 190 | NKSGNRSEKEVRAAALVLQTIWGYK | CTD1_HUMAN |
| 191 | RHLTQQDPLSEAIVEKLIQSIQKVF | CTDB_HUMAN |
| 192 | RFVKLAWMGLTVALGAAALAVVKSA | CTE0_HUMAN |
| 193 | SVKRGNMVRAARALLSAVTRLLILA | CTN1_HUMAN |
| 194 | SVKRGTMVRAARALLSAVTRLLILA | CTN2_HUMAN |
| 195 | ARENAGPAIVISFLIAALASVLAGL | CTR1_HUMAN |
| 196 | VSSSAPSVYSVQALSLLAEVLASLL | CU05_HUMAN |
| 197 | VYRSDEKEKAVPLISRLLYYVFPYL | CU05_HUMAN |
| 198 | RGPHGQLSPALPLASSVLMLLMSTL | CV03_HUMAN |
| 199 | TLRFLHASALLALASGLLAVLLAGL | CV03_HUMAN |
| 200 | FPNPRRRLRLQDLADRVVDASEDEH | CYA3_HUMAN |
| 201 | LHYYSEREGLQDIVIGIIKTVAQQI | CYG1_HUMAN |
| 202 | WKGAPTTSLISVAVTKIIAKVLEDN | D7A1_HUMAN |
| 203 | YVASAFTLAVNIIAKKIVLKRQTGS | DCOR_HUMAN |
| 204 | LLRILTDALVPYLVGQVVAGAQALQ | DCUP_HUMAN |
| 205 | AEKTDEEEKEDRAAQSLLNKLIRSN | DD19_HUMAN |
| 206 | DEERRQLIQLRDILKSALQVEQKED | DDF2_HUMAN |
| 207 | LGHPAAFGRATHAVVRALPESLGQH | DDH1_HUMAN |
| 208 | ASFQRKWFEVAFVAEELVHSEIPAF | DEP5_HUMAN |
| 209 | KVAFTGSTEVGKLIKEAAGKSNLKR | DHA1_HUMAN |
| 210 | KIAFTGSTEVGKLIQEAAGRSNLKR | DHA2_HUMAN |
| 211 | EAIKFINRQEKPLALYAFSNSRQVV | DHA8_HUMAN |
| 212 | PRALLAALWALEAAGTAALRIGAFN | DHP1_HUMAN |
| 213 | RLVSSPPSGVPGLALLALLALLALR | DIAC_HUMAN |
| 214 | RVVLKGDVSLKDIIDPAFRASWIAQ | DIAC_HUMAN |
| 215 | FQLPSRQPALSSFLGHLAAQVQAAL | DIS1_HUMAN |
| 216 | RSLTSEREGLEGLLSKLLVLSSRNV | DIS1_HUMAN |
| 217 | LIGPKVRITLMKFLPSVFMDAMRDN | DJCD_HUMAN |
| 218 | QRPRAPRSALWLLAPPLLRWAPPLL | DLG4_HUMAN |
| 219 | RQRLLGRSWSVPVIRHLFAPLKEYF | DM3A_HUMAN |
| 220 | KRKLEDLSSEWKAVNRLLQELRAKQ | DMD_HUMAN |
| 221 | LPARVPRPGLSEALSLLLFAVVLSR | DMK_HUMAN |
| 222 | QRELQEALGARAALEALLGRLQAER | DMN_HUMAN |
| 223 | SARLRMVETLSNLLRSVVALSPPDL | DNL1_HUMAN |
| 224 | ELAEHLNASLAFFLSDLLSLVDRGF | DOC6_HUMAN |
| 225 | GPFRQQHFLAGLLLTELALALEPEA | DOC6_HUMAN |
| 226 | LRAHGTHPAISTLARSAIFSVTYPS | DOC6_HUMAN |
| 227 | IYEPPRYMSVNQAAQQLLEIVQNQR | DPH5_HUMAN |
| 228 | HIYLYHHAQAHKALFGIFIPSQRRA | DPOE_HUMAN |
| 229 | FNWRQAHMQARFVILRVLLEAGEGL | DPP3_HUMAN |
| 230 | GVILGKWAILAILLGIALLFSVLLT | DSC3_HUMAN |
| 231 | PNTELNVSRLEAVLSTIFYQLNKRM | DTNA_HUMAN |
| 232 | RLDEEHRLIARYAARLAAESSSSQP | DTNA_HUMAN |
| 233 | LTSVLGILASSTVLFMLFRPLFRWQ | DUFF_HUMAN |
| 234 | PQELYESSHIESAINVAIPGIMLRR | DUS6_HUMAN |
| 235 | SRELYESARIGGALSVALPALLLRR | DUS9_HUMAN |
| 236 | DYYKKQVAQLKTLITMLIGQLSKGD | DYH9_HUMAN |
| 237 | RDFVEEKLGSKYVVGRALDFATSFE | DYH9_HUMAN |
| 238 | IGVKFLINEATTLADLLALRLHRVE | DYHB_HUMAN |
| 239 | EGKKKQTNYLRTLINELVKGILPRS | DYHC_HUMAN |
| 240 | GPSGSGKSMAWRVLLKALERLEGVE | DYHC_HUMAN |
| 241 | KLVAEDIPLLFSLLSDVFPGVQYHR | DYHC_HUMAN |
| 242 | LLSATELDKIRQALVAIFTHLRKIR | DYHC_HUMAN |
| 243 | WRRFRWAIILFIILFILLLFLAIFT | DYSF_HUMAN |
| 244 | PVRREVTDKEQSFAARAAKQLEYQQ | E4L3_HUMAN |
| 245 | FPGDILMRMLKMLTLPLTISSLITG | EAA2_HUMAN |
| 246 | KLMVDFFNILNEIVMKLVIMIMWYS | EAA2_HUMAN |
| 247 | FPGEILMRMLKLIILPLIISSMITG | EAA3_HUMAN |
| 248 | PFMSAVSGRAYPAAITILETAQKIA | EDD_HUMAN |
| 249 | RNTFAERLSAVEAIANAISVVSSNG | EDD_HUMAN |
| 250 | NAAQTPRIPSRLLAILLFLLAMLLT | EFA5_HUMAN |
| 251 | GLKELPMRNLQEILHGAVRFSNNPA | EGFR_HUMAN |
| 252 | MVETQQLMRVYGALMWALGKVVGTP | EHD2_HUMAN |
| 253 | HGIVSWDTFSVAFIKKIASFVNKSA | ELM1_HUMAN |
| 254 | HGIVSWDMVSITFIKQIAGYVSQPM | ELM2_HUMAN |
| 255 | VMNQQLQTKAMALLTALLQGASPVE | ELM3_HUMAN |
| 256 | ESRVQQQEDEITVLKAALADVLRRL | EML4_HUMAN |
| 257 | GQFGVGFYSAFLVADKVIVTSKHNN | ENPL_HUMAN |
| 258 | ELSSQLPERLSLVIGSILGALAFLL | EPB6_HUMAN |
| 259 | MYRTHTRRALQTVAQLILELIEKQE | EPPL_HUMAN |
| 260 | SGRAAALRQVVSAVTALVEAAERQP | EPPL_HUMAN |
| 261 | RLQALRLEREEVVLLKALALANSDS | ERR1_HUMAN |
| 262 | GESAGGESVSVLVLSPLAKNLFHRA | EST1_HUMAN |
| 263 | TPQKNNYNSIAAILIGVLLTSMLVA | EV2B_HUMAN |
| 264 | ALRPAPALLAPAVLLGAALGLGLGL | EVC_HUMAN |
| 265 | NILVTTTQLIPALAKVLLYGLGIVF | EYA1_HUMAN |
| 266 | NVLVTTTQLIPALAKVLLYGLGSVF | EYA2_HUMAN |
| 267 | PADEKLQEKAWGAVVPLVGKLKKFY | F49B_HUMAN |
| 268 | VEQRKKLSSLLEFAQYLLAHSMFSR | FACA_HUMAN |
| 269 | NRLGIESPRSEKLARELLKELRTQV | FACC_HUMAN |
| 270 | QQRAQTMVQVKAVLGHLLAMSRSSS | FACC_HUMAN |
| 271 | SGQSKLNSWIQGVLSHILSALRFDK | FACC_HUMAN |
| 272 | ETRRGAYLNALKIAKLLLTAIGYGH | FAFX_HUMAN |
| 273 | SQAYDNLSLSDHLLRAVLNLLRREV | FAFX_HUMAN |
| 274 | WVVPVLPKGELEVLLEAAIDLSKKG | FAFX_HUMAN |
| 275 | WVVPVLPKGELEVLLEAAIDLSVKG | FAFY_HUMAN |
| 276 | RSNDKVYENVTGLVKAVIEMSSKIQ | FAK1_HUMAN |
| 277 | ARTSSAEYNVNNLVSPVLFQEALWH | FAS_HUMAN |
| 278 | FRYMAQGKHIGKVVVQVLAEEPAVL | FAS_HUMAN |
| 279 | AVTIHPVTGSISVLNPAFLGLSRKL | FAT2_HUMAN |
| 280 | PGPAPLRLLEWRVAAGAAVRIGSVL | FCP1_HUMAN |
| 281 | MEEWDRYPRIGDILQKLAPFLKMYG | FGD1_HUMAN |
| 282 | SGTKKSDFHSQMAVHKLAKSIPLRR | FGR1_HUMAN |
| 283 | PSHSLLRLPLLQLLLLVVQAVGRGL | FK10_HUMAN |
| 284 | DVFIWLGRKSPRLVRAAALKLGQEL | FLIH_HUMAN |
| 285 | HRLLQQLVLSGNLIKEAVRRLQRAV | FRT2_HUMAN |
| 286 | LPHGSGLGTSSILAGTALAALQRAA | FUK_HUMAN |
| 287 | PESTARMQGAGKALHELLLSAQRQG | G45G_HUMAN |
| 288 | PKVDKWSRFLFPLAFGLFNIVYWVY | GAAT_HUMAN |
| 289 | RLFTNLKDTSSKVTQSVANYAKGDL | GAK_HUMAN |
| 290 | ESLREVQLEELEAARDLVSKEGFRR | GAL1_HUMAN |
| 291 | RPFLPYFNVSQQFATFVLKGSFSEI | GALC_HUMAN |
| 292 | NHGMWQTISVEELARNLVIKVNRDA | GAS6_HUMAN |
| 293 | AGEDPKVTRAKFFIRDLFLRISTAT | GBAF_HUMAN |
| 294 | PSVFGSNPKAHIAAKTVFHLAHRHG | GBF1_HUMAN |
| 295 | HGRLIFITVLFSIIIWVVWISMLLR | GC5D_HUMAN |
| 296 | NVLKDKMEKLKRLLQVAARKSQVTL | GCC1_HUMAN |
| 297 | TINKFDKNFSAHLLDLLARLSIYST | GCP2_HUMAN |
| 298 | IINNDTTITLAIVVDKLAPRLSQLK | GCP5_HUMAN |
| 299 | GLLTEKAAPVMNVIHSIFSLVLKFR | GCP6_HUMAN |
| 300 | IAKQELIAHAREAASRVLSALSDRQ | GCP6_HUMAN |
| 301 | SYESMSEPPIAHLLRPVLPRAFAFP | GCP6_HUMAN |
| 302 | GSAVEIVGLSKSAVRWLLELSKKNI | GDE_HUMAN |
| 303 | LRLETAPNISKDVIRQLLPKAPPLR | GDF8_HUMAN |
| 304 | VLGDIHTTLLSAVIPNAFRLVKRKP | GDL1_HUMAN |
| 305 | KDHAGVMGESNRLLSALIRHSKSKD | GDS1_HUMAN |
| 306 | FNQLTQSASEQGLAKAVASVARLVI | GEM4_HUMAN |
| 307 | GRQKLARFNAREFATLIIDILSEAK | GIT1_HUMAN |
| 308 | KPVYYALEGSVAIAGAVIRWLRDNL | GLPK_HUMAN |
| 309 | VGILSRRLQEALAAKEAADAELGQL | GOA3_HUMAN |
| 310 | FLRRYPIARVFVIIYMALLHLWVMI | GOA5_HUMAN |
| 311 | VLGLFWLLFASVVLILLLSWVGHVK | GPBA_HUMAN |
| 312 | QASWVRPGVLWDVALVAVAALGLAL | GPIX_HUMAN |
| 313 | FRLVSRRDYASEAIKGAVVGIDLGT | GR75_HUMAN |
| 314 | EDFKAKKKELEEIVQPIISKLYGSA | GR78_HUMAN |
| 315 | GQPKRYKGFSIDVLDALAKALGFKY | GRD1_HUMAN |
| 316 | FQGKKNMTLAGRLAGPLFQTLIVAW | GRIP_HUMAN |
| 317 | QKGHKSQREELDAILFIFEKILQLL | GRIP_HUMAN |
| 318 | VKTQMQHGLTSIAARTVITHLVNHL | GRIP_HUMAN |
| 319 | VPTWDTIRDEEDVLDELLQYLGVTS | GRIP_HUMAN |
| 320 | HEHIERRRKLYLAALPLAFEALIPN | GRLF_HUMAN |
| 321 | GVWSEKGQVEVFALRRLLQVVEEPQ | GRWD_HUMAN |
| 322 | MRPEDRMFHIRAVILRALSLAFLLS | HA2Q_HUMAN |
| 323 | SHTRGPEQQVKAILSELLQRENRVL | HAPI_HUMAN |
| 324 | AMSKSRNPRLQTAAQELLEDLRTLE | HBP2_HUMAN |
| 325 | ALGHKRNSGVPAFLTPLLRNIIISL | HD_HUMAN |
| 326 | ENEDKWKRLSRQIADIILPMLAKQQ | HD_HUMAN |
| 327 | FGDAALYQSLPTLARALAQYLVVVS | HD_HUMAN |
| 328 | GLLKLQERVLNNVVIHLLGDEDPRV | HD_HUMAN |
| 329 | NLSSRETSSLESFVRRVANIARTNA | HED1_HUMAN |
| 330 | GLPRPPMLLALLLATLLAAMLALLT | HEXB_HUMAN |
| 331 | DQRKMLLVGSRKAAEQVIQDALNQL | HIP1_HUMAN |
| 332 | GIALAYGSLLLMALLPIFFGALRSV | HM13_HUMAN |
| 333 | PNWKLKVSNLKMVLRSLVEYSQDVL | HOK2_HUMAN |
| 334 | GDQLAQLNTVFQALPTAAWGATLRA | HPS6_HUMAN |
| 335 | LLSSGRPKAVLQAVGQLVQKEQWDR | HPS6_HUMAN |
| 336 | QKHAQQQKVVNKLIQFLISLVQSNR | HSF1_HUMAN |
| 337 | AKHAQQQQVIRKIVQFIVTLVQNNQ | HSF2_HUMAN |
| 338 | LNAARRYLGIEDLAGKVFVTSGLGG | HUTU_HUMAN |
| 339 | FEKMISGMYLGEIVRHILLHLTSLG | HXK3_HUMAN |
| 340 | IPANLSQNLEAAAATQVAVSVPKRR | I4G1_HUMAN |
| 341 | IFHKNVFHYLMAFLRELLKNSAKNH | I5P2_HUMAN |
| 342 | FEQWAHSEDLQSLLLRVANAVSVKG | ICE9_HUMAN |
| 343 | KNSEATLPIAVRFAKTLLANSSPFN | IF_HUMAN |
| 344 | SGKVSADNTVGRFLMSLVNQVPKIV | IF35_HUMAN |
| 345 | NPKIWNVHSVLNVLHSLVDKSNINR | IF31_HUMAN |
| 346 | LHGVMEYDLSLRFLENALAVSTKYH | IF3X_HUMAN |
| 347 | ENGMYGKRKLLELIGHAVAHLKKAD | IFT2_HUMAN |
| 348 | YRRKDEPDKAIELLKKALEYIPNNA | IFT2_HUMAN |
| 349 | APSDPDAVSAEEALKYLLHLVDVNE | IKAP_HUMAN |
| 350 | QLVKLLGASELPIVTPALRAIGNIV | IMA2_HUMAN |
| 351 | SSNVENQLQATQAARKLLSREKQPP | IMA2_HUMAN |
| 352 | PLLSHQEVKVQTAALRAVGNIVTGT | IMA4_HUMAN |
| 353 | MRKEEPSNNVKLAATNALLNSLEFT | IMB1_HUMAN |
| 354 | PPEHTSKFYAKGALQYLVPILTQTL | IMB1_HUMAN |
| 355 | PYYDLFMPSLKHIVENAVQKELRLL | IMB3_HUMAN |
| 356 | TAAEEARQMAAVLLRRLLSSAFDEV | IMB3_HUMAN |
| 357 | LIAYRSRKRLETFLSLLVQNLAPAE | INPP_HUMAN |
| 358 | FVEPHKNMEVMGFLHGIFERLKQFL | IP11_HUMAN |
| 359 | LPVPQGPNPVVVVLQQVFQLIQKVL | IP13_HUMAN |
| 360 | NRERQKLMREQNILKQIFKLLQAPF | IP3R_HUMAN |
| 361 | NRERQKLMREQNILAQVFGILKAPF | IP3S_HUMAN |
| 362 | EKRVADPTLEKYVLSVVLDTINAFP | IP3T_HUMAN |
| 363 | NRERQKLMREQNILKQVFGILKAPF | IP3T_HUMAN |
| 364 | ETIQGLGAASAQFVSRLLPVLLSTA | IPO4_HUMAN |
| 365 | HPAQEHFPKLLGLLFPLLARERHDR | IPO4_HUMAN |
| 366 | LLRNPSSPRAKELAVSALGAIATAA | IPO4_HUMAN |
| 367 | LMASPTRKPEPQVLAALLHALPLKE | IPO4_HUMAN |
| 368 | SKALLKNRLLPPLLHTLFPIVAAEP | IPO4_HUMAN |
| 369 | YMQAVNRERERQVVMAVLEALTGVL | IPO4_HUMAN |
| 370 | DQYRQKEYVAPRVLQQAFNYLNQGV | IPO8_HUMAN |
| 371 | ILADLNLSVSPFLLGRALWAASRFT | IPO9_HUMAN |
| 372 | LGFENLVFSIFEFVHALLENSKFKS | IPO9_HUMAN |
| 373 | PERWTNIPLLVKILKLIINELSNVM | IPO9_HUMAN |
| 374 | RTSEFTAAFVGRLVSTLTSKAGREL | IPO9_HUMAN |
| 375 | LYNYASNQREEYLLLRLFKTALQEE | IQG1_HUMAN |
| 376 | NRGARGQNALRQILAPVVKEIMDDK | IQG1_HUMAN |
| 377 | YQDLLQLQYEGVAVMKLFDRAKVNV | IQG1_HUMAN |
| 378 | LYNYASNQREEYLLLKLFKTALEEE | IQG2_HUMAN |
| 379 | NRGARGQNTLRQLLAPVVKEIIDDK | IQG2_HUMAN |
| 380 | WSPRKLPSSASTFLSPAFPGSQTHS | IRA1_HUMAN |
| 381 | WLGGGVVPDAIVLAEEALDKAQEVL | IRBP_HUMAN |
| 382 | GKPLERKLILVQVIPVVARMTYEMF | IRF6_HUMAN |
| 383 | WRYMLLIFSLAFLASWLLFGIIFWV | IRKC_HUMAN |
| 384 | WRWMMLVFSASFVVHWLVFAVLWYV | IRKD_HUMAN |
| 385 | ALVIFEMPHLRDVALPALGAVLRGA | IRR_HUMAN |
| 386 | VGWIIAISLLVGILIFLLLAVLLWK | ITA9_HUMAN |
| 387 | SNSIYPWSEVQTFLRRLVGKLFIDP | ITAG_HUMAN |
| 388 | PIWIIVGSTLGGLLLLALLVLALWK | ITAH_HUMAN |
| 389 | QGFTYTATAIQNVVHRLFHASYGAR | ITAX_HUMAN |
| 390 | ARPRPRPLWVTVLALGALAGVGVGG | ITB3_HUMAN |
| 391 | LVVLLSVMGAILLIGLAALLIWKLL | ITB3_HUMAN |
| 392 | MAGPRPSPWARLLLAALISVSLSGT | ITB4_HUMAN |
| 393 | AARQRQEIAAARAADALLKAVAASS | JPH4_HUMAN |
| 394 | LSTLRYADRAKRIVNHAVVNEDPNA | K13A_HUMAN |
| 395 | SNINKSLTTLGLVISSLADQAAGKG | K13A_HUMAN |
| 396 | LSTLRYADRAKHIVNNAVVNEDPNA | K13B_HUMAN |
| 397 | QENLSLIGVANVFLESLPYDVKLQY | K13B_HUMAN |
| 398 | STSFRGGMGSGGLATGIAGGLAGMG | K1CR_HUMAN |
| 399 | GFDVNIVEEELGIISRAVKHLFKSI | K21A_HUMAN |
| 400 | HQSVVYRKQAAMILNELVTGAAGLE | K406_HUMAN |
| 401 | SLVHRLTRDAPLAVLRAFKVLRTLG | K406_HUMAN |
| 402 | YLSVKQPVKLQEAARSVFLHLMKVD | K406_HUMAN |
| 403 | KNVMEFLAPLKPVAIRIVRNAHGNK | K682_HUMAN |
| 404 | GMRRGNMGHLTRIANAVVQNLERGP | K685_HUMAN |
| 405 | RQDVLHWLNEEKVIQRLVELIHPSQ | K685_HUMAN |
| 406 | EELVSIPWKVLKVVAKVIRALLRIL | K830_HUMAN |
| 407 | GFTATPFIKLFQLIYYLVVAILKNT | K830_HUMAN |
| 408 | TETLQHPERARDALRVLLHLVEKSL | KA43_HUMAN |
| 409 | RQDVVNWLNEEKIVQRLIEQIHPSK | KB15_HUMAN |
| 410 | ALHLATEMEELGLVTHLVTKLRANV | KBF2_HUMAN |
| 411 | NSQWRQDMSISLAALELLSGLAKVK | KC19_HUMAN |
| 412 | EKGFYTERDASRLIFQVLDAVKYLH | KCC1_HUMAN |
| 413 | EKGYYSERDAADAVKQILEAVAYLH | KCC4_HUMAN |
| 414 | LQEFNARRKLKAAVKAVVASSRLGS | KCC4_HUMAN |
| 415 | YALKRSFKELGLLLMYLAVGIFVFS | KCF1_HUMAN |
| 416 | LLRLASTPDLRRFARSALNLVDLVA | KCV2_HUMAN |
| 417 | STYFDMNLFLDIILKTVLENSGKRR | KE34_HUMAN |
| 418 | RGGVVRQYWSSSFLVDLLAVAAPVV | KE72_HUMAN |
| 419 | SALESTEEKLHDAASKLLNTVEETT | KF11_HUMAN |
| 420 | QENEPTVGVIPRVIQLLFKEIDKKS | KF4A_HUMAN |
| 421 | ELSNHQKKRATEILNLLLKDLGEIG | KF5C_HUMAN |
| 422 | EATAFGLGKEDAVLKVAVKMLKSTA | KFMS_HUMAN |
| 423 | KDKLKPGAAEDDLVLEVVIMIGTVS | KFP3_HUMAN |
| 424 | MEGKLHDPQLMGIIPRIARDIFNHI | KINN_HUMAN |
| 425 | SSAIIDHIFASKAVVNAAIPAYHLR | KIST_HUMAN |
| 426 | YLEVGLSGLSSKAAKDVLGFLRVVR | KNC1_HUMAN |
| 427 | LEKLGYMDLASRLVTRVFKLLQNQI | LCAP_HUMAN |
| 428 | EAPAYHLILEGILILWIIRLLFSKT | LCB1_HUMAN |
| 429 | KTPSGIKLTINKLLDMAAQIAEGMA | LCK_HUMAN |
| 430 | ARGYVQDPFAALLVPGAARNAPLIE | LCM2_HUMAN |
| 431 | DSLYFRLKTAGRLARAAVWEVDFPD | LCM2_HUMAN |
| 432 | PLPSLRFLEELRLAGNALTYIPKGA | LGR5_HUMAN |
| 433 | FWKAFWNITEMEVLSSLANMASATV | LIPS_HUMAN |
| 434 | VTITLDLRQVFQVAYVIIKAANAPR | LMA1_HUMAN |
| 435 | ERTNTRAKSLGEFIKELARDAEAVN | LMA2_HUMAN |
| 436 | ETQKEIAEDELVAAEALLKKVKKLF | LMA2_HUMAN |
| 437 | QSQAHQQRGLFPAVLNLASNALITT | LMA2_HUMAN |
| 438 | NSARDAVRNLTEVVPQLLDQLRTVE | LMA4_HUMAN |
| 439 | VKLSNLSNLSHDLVQEAIDHAQDLQ | LMA4_HUMAN |
| 440 | GQPLPWELRLGLLLSVLAATLAQAP | LMB2_HUMAN |
| 441 | ALDEKVRERLRMALERVAVLEEELE | LPA3_HUMAN |
| 442 | DNTKSQLAMSANFLGSVLTLLQKQH | LPC4_HUMAN |
| 443 | LLGGIKVKLLRGLLPNLVDNLVNRV | LPC4_HUMAN |
| 444 | KGNKSSYHRLSELVEHVFPLLSKEQ | LPN2_HUMAN |
| 445 | KNHKSTYERLGEVVELLFPPVARGP | LPN3_HUMAN |
| 446 | TVLDQQQTPSRLAVTRVIQALAMKG | LPRC_HUMAN |
| 447 | PGPLPSLPLEPSLLSGVVQALRGRL | LR10_HUMAN |
| 448 | SKTEQPAALALDLVNKLVYWVDLYL | LR1B_HUMAN |
| 449 | AAKYRDHVTATQLIQKIINILTDKH | LRBA_HUMAN |
| 450 | VSNMSITERLEHALEKAAPLLREIF | LRBA_HUMAN |
| 451 | LSLLLLVTSVTLLVARVFQKAVDQS | LYII_HUMAN |
| 452 | DHLSQSKVIETQLAKPLFDALLRVA | LYST_HUMAN |
| 453 | SQAELVQKGSELVALRVALREARAT | LZT2_HUMAN |
| 454 | SRHHHGSSIAGGLVKGALSVAASAY | M172_HUMAN |
| 455 | GMAAGLYSELFTLLVSLVNRALKSS | M18A_HUMAN |
| 456 | AVVFSYIATLLYVVHAVFSLIRWKS | MAL_HUMAN |
| 457 | PPLNTIRDVSLKIAEKIVKDAYQEK | MAOX_HUMAN |
| 458 | TFNDDIQGTASVAVAGLLAALRITK | MAOX_HUMAN |
| 459 | KRKTTAAGGESALAPSVFKQAKDKV | MAP2_HUMAN |
| 460 | KGKIKVIKKEGKAAEAVAAAVGTGA | MAPB_HUMAN |
| 461 | FHPAVRNSSEVKFAVQAFAALNSNN | MC3A_HUMAN |
| 462 | RRLGGLASQEPGAIIELFNSVLQFL | MC3A_HUMAN |
| 463 | PPAAARAGGSPTAVRSILTKERRPE | MCDL_HUMAN |
| 464 | RFSVVDMAALGGVLGALLLLALLGL | MCDL_HUMAN |
| 465 | RGTVARGAGAGVVVKDAAAPSQPLR | MCDL_HUMAN |
| 466 | AAFMKKYIHVAKIIKPVLTQESATY | MCM3_HUMAN |
| 467 | DLRRKNEKRANRLLNNAFEELVAFQ | MCM3_HUMAN |
| 468 | PFPSWRFPGLLLAAMVLLLYSFSDA | MCP_HUMAN |
| 469 | DVSTLHVQKIISAISELLERLKSYG | MDN1_HUMAN |
| 470 | LATHRSTAKLLSVLAQVFTELAQKG | MDN1_HUMAN |
| 471 | SLRNFYSHSLSGAVSNVFKILQPNT | MDN1_HUMAN |
| 472 | VTSIAKAPAVQDLLTRLLQALHIDG | MDN1_HUMAN |
| 473 | QQLQKQLKEAEQILATAVYQAKEKL | MED4_HUMAN |
| 474 | RLRHWWAIALTTAVTSAFLLAKVIL | MENT_HUMAN |
| 475 | MNMAKTSQTVATFLDELAQKLKPLG | MEPD_HUMAN |
| 476 | KDTKVDRSAAYAARWVAKSLVKGG | METK_HUMAN |
| 477 | KDYTKVDRSAAYAARWVAKSLVKAG | METL_HUMAN |
| 478 | VSPLKHFVLAKKAITAIFDQLLEFV | MFN1_HUMAN |
| 479 | GYFPMIFRKAREFIEILFGISLTEV | MGC3_HUMAN |
| 480 | RKPRFMSAWAQVIIASILISVQLTL | MGR1_HUMAN |
| 481 | LKTAADRQAEDQVLRKLVDLVNQRD | MIL1_HUMAN |
| 482 | YLMVMIGMFSFIIVAILVSTVKSKR | MIR1_HUMAN |
| 483 | SSQPLTLEHVRYFLYQLLRGLKYMH | MK07_HUMAN |
| 484 | GARLALDEHVQFLVYQLLRGLKYIH | MK11_HUMAN |
| 485 | VKKPAGPSISKPAAKPAAAGAPPAK | MLEY_HUMAN |
| 486 | RGERLHMFRVGGLVFHAIGQLLPHQ | MLL2_HUMAN |
| 487 | WYVLVIISDLMTIIGSILKMEIKAK | MLN2_HUMAN |
| 488 | SGKPRRKSNLPIFLPRVAGKLGKRP | MLPH_HUMAN |
| 489 | KQKHFNEREASRVVRDVAAALDFLH | MNK1_HUMAN |
| 490 | KHLERRDAESLKLLKEAIWEEKQGT | MOC3_HUMAN |
| 491 | VYRKMPEHVLGRIAVAVVKGLTYLW | MPK5_HUMAN |
| 492 | DLIFLRGIMESPIVRSLAKVIMVLW | MPP2_HUMAN |
| 493 | STATNPQNGLSQILRLVLQELSLFY | MPP4_HUMAN |
| 494 | ARRRLWGFSESLLIRGAAGRSLYFG | MPPB_HUMAN |
| 495 | YYAKAFSKDLPRAVEILADIIQNST | MPPB_HUMAN |
| 496 | HSVMDTLAVALRVAEEAIEEAISKA | MRIP_HUMAN |
| 497 | AVSSAHRAASLEAVSYAIDTLKAKV | MS2L_HUMAN |
| 498 | DEKKRRLMGLPSFLTEVARKELENL | MSH5_HUMAN |
| 499 | SPSLQEKLKSFKAALIALYLLVFAV | MSRE_HUMAN |
| 500 | SVRPLEFTKVKTFVSRIIDTLDIGP | MTN3_HUMAN |
| 501 | SVRPQNFELVKRFVNQIVDFLDVSP | MTN4_HUMAN |
| 502 | EEPYRRRNQILSVLAGLAAMVGYAL | MTX1_HUMAN |
| 503 | VQLRRGLVGSRPVVTRVVIKAQGLV | MU5B_HUMAN |
| 504 | LVDKGTEDKVVDVVRNLVFHLKKGY | MX1_HUMAN |
| 505 | KITINSHTTAGEVVEKLIRGLAMED | MY10_HUMAN |
| 506 | AGKQGLGPPSTPIAVHAAVKSKSLG | MYCD_HUMAN |
| 507 | VSSTVGAAAVSALAGGALNGVFGRR | MYCT_HUMAN |
| 508 | GKTVNTKRVIQYFATIAVTGEKKKE | MYH4_HUMAN |
| 509 | VTKGQTVQQVYNAVGALAKAVYDKM | MYH1_HUMAN |
| 510 | VTKGQTVEQVSNAVGALAKAVYEKM | MYH2_HUMAN |
| 511 | VTKGQTVQQVYNAVGALAKAIYEKM | MYH4_HUMAN |
| 512 | GKTVNTKRVIQYFATIAVTGEKKKD | MYH8_HUMAN |
| 513 | VTKGQTVQQVYNAVGALAKAVYEKM | MYH8_HUMAN |
| 514 | EQLKRNSQRAAEALQSVLDAEIRSR | MYHD_HUMAN |
| 515 | QAAVQLALRAGQIIRKALTEEKRVS | MYO2_HUMAN |
| 516 | IVWRRFKWVIIGLLFLLILLLFVAV | MYOF_HUMAN |
| 517 | PLDDLDREDEVRLLKYLFTLIRAGM | N107_HUMAN |
| 518 | IVLKNHHSRLSDLVNTAILIALNKR | N133_HUMAN |
| 519 | HEAQLSEKISLQAIQQLVRKSYQAL | N155_HUMAN |
| 520 | QGMSRVASVSQNAIVSAAGNIARTI | N155_HUMAN |
| 521 | YRRAAEKWEVAEVVLEVFYKLLRDY | N205_HUMAN |
| 522 | LFTFQKHVFSPIFIIGAFVAIFLGR | NAH7_HUMAN |
| 523 | TSGRRWREISASLLYQALPSSPDHE | NAL1_HUMAN |
| 524 | AEKQPPFTLIRSLLRKVLLPESFLT | NAL5_HUMAN |
| 525 | AENMSITAKLERALEKVAPLLREIF | NBEA_HUMAN |
| 526 | STVKIQNPMILKVVATLLKNSTPSA | NBEA_HUMAN |
| 527 | KGGVGKSTFSAHLAHGLAEDENTQI | NBP1_HUMAN |
| 528 | FIQEFPGSPAFAALTSIAQKILDAT | NBP2_HUMAN |
| 529 | WRGPKKNALIKQFVSDVAWGELDYL | NBP2_HUMAN |
| 530 | SARDLQNLMSWRFIMDLVSSLSRTY | NEP_HUMAN |
| 531 | SKLPKDQQDAKHILEHVFFQVVEFK | NGD5_HUMAN |
| 532 | EPEPITASGSLKALRKLLTASVEVP | NIBA_HUMAN |
| 533 | GRVLYREDTSPAVLGLAARYVRAGF | NID2_HUMAN |
| 534 | RTKRLHWSRLLFLLGMLIIGSTYQH | NKX1_HUMAN |
| 535 | YKFGSRHSAESQILKHLLKNLFKIF | NNMT_HUMAN |
| 536 | IVDNGVAPPARRLLRLVVFRAPQVE | NPHN_HUMAN |
| 537 | GPVSARVIKALQVVDHAFGMLMEGL | NPP3_HUMAN |
| 538 | SPESDAPGPVYAAASLAVSWVLRSV | NPR1_HUMAN |
| 539 | SPPALPLASSFTALLQAAYESQALR | NPR1_HUMAN |
| 540 | TMPHLSMQQVLLAAKQVLLYLRSTV | NPR1_HUMAN |
| 541 | TQDMRLTFTLALFIAKAALQILKPE | NPR1_HUMAN |
| 542 | VLQQRLGELERQLLRKVAELEDEKS | NPX2_HUMAN |
| 543 | ISPLIQKSAANVVLFDIFVNILTHN | NRDC_HUMAN |
| 544 | VQGNVTSTESMDFLKYVVDKLNFKP | NRDC_HUMAN |
| 545 | GFKLLWILLLATLVGLLLQRLAARL | NRM2_HUMAN |
| 546 | VYVRDLGHVALYVVAAVVSVAYLGF | NRM2_HUMAN |
| 547 | RKPGNVLKTLEPILITIIAMSALGV | NRP1_HUMAN |
| 548 | DYVPIGPRFSNLVLQALLVLLKKAP | NSF_HUMAN |
| 549 | YMIHHGDWFSGKAVGLLVLHLSGMV | NSMA_HUMAN |
| 550 | ILWSGWASNSNYALIGALRAVAQTI | NU1M_HUMAN |
| 551 | IRFLKGMGYSTHAAQQVLHAASGNL | NUB1_HUMAN |
| 552 | KQIQRTKRGLEILAKRAAETVVDPE | NUB1_HUMAN |
| 553 | ELLNMYVGESERAVRQVFQRAKNSA | NVL_HUMAN |
| 554 | RLSLVAGAYVAGLISALVRTVSAFT | O9O1_HUMAN |
| 555 | WRRLPGAGLARGFLHPAATVEDAAQ | ODBB_HUMAN |
| 556 | IVFNAHIKGVETIANDVVSLATKAR | ODP2_HUMAN |
| 557 | DFLNNPFKQENVLARMVASRITNYP | OFD1_HUMAN |
| 558 | NLRRDVYIRIASLLKTLLKTEEWVL | OFU2_HUMAN |
| 559 | AAETLLSSLLGLLLLGLLLPASLTG | OS9_HUMAN |
| 560 | KRENPQLKQIEGLVKELLEREGLTA | OS9_HUMAN |
| 561 | KRVAYARVPSKDLLFSIVEEETGKD | OTOF_HUMAN |
| 562 | TVPVFFNQAERRAVLQAARMAGLKV | OXRP_HUMAN |
| 563 | VGGATRVPRVQEVLLKAVGKEELGK | OXRP_HUMAN |
| 564 | DQKAYKEGKLQKALEDAFLAIDAKL | P2CG_HUMAN |
| 565 | TKYKMGGDIANRVLRSLVEASSSGV | P2G4_HUMAN |
| 566 | NRPSIPYAFSKFLLPIVVRYLADQN | P4R1_HUMAN |
| 567 | HRSPQLLLELDNVISVLFQNSKERG | P52K_HUMAN |
| 568 | HRHMRTIREVRTLVTRVITDVYYVD | P531_HUMAN |
| 569 | AEQFAPPDIAPPLLIKLVEAIEKKG | P85A_HUMAN |
| 570 | PQVQETLNLEPDVAQHLLAHSHWGA | PARC_HUMAN |
| 571 | VAMGEMEADVQALVRRAARQLAESG | PARC_HUMAN |
| 572 | VELLTNQVGEKMVVVQALRLLYLLM | PARC_HUMAN |
| 573 | HPPYLVSKELMSLVSGLLQPVPERR | PASK_HUMAN |
| 574 | STMSPLGSGAFGFVWTAVDKEKNKE | PASK_HUMAN |
| 575 | TSRRRNVTFSQQVANILLNGVKYES | PAST_HUMAN |
| 576 | LFTLDEQSGLLTVAWPLARRANSVV | PC16_HUMAN |
| 577 | KVTDHGKPTLSAVAKLIIRSVSGSL | PC17_HUMAN |
| 578 | SHINAATGTSASLVYRLVSKAGDAP | PCH9_HUMAN |
| 579 | NGETLVFEESNWFIINVIKLVWRYG | PCL1_HUMAN |
| 580 | KADVNLSHSERGALQDALRRLLGLF | PCN2_HUMAN |
| 581 | NQRKAAHSAELEAVLLALARIRRAL | PCN2_HUMAN |
| 582 | ISVQLKKTSEVDLAKPLVKFIQQTY | PD6I_HUMAN |
| 583 | NRSIAQMREATTLANGVLASLNLPA | PD6I_HUMAN |
| 584 | PAKTMQGSEVVNVLKSLLSNLDEVK | PD6I_HUMAN |
| 585 | VTEFNSQTSAKIFAARILNHLLLFV | PDA2_HUMAN |
| 586 | RLALFPGVALLLAAARLAAASDVLE | PDA3_HUMAN |
| 587 | YQGGRTGEAIVDAALSALRQLVKDR | PDA6_HUMAN |
| 588 | FSEMLAASFSIAVVAYAIAVSVGKV | PEND_HUMAN |
| 589 | THKIPVPIPIEVIVTIIATAISYGA | PEND_HUMAN |
| 590 | ELLSKYIGASEQAVRDIFIRAQAAK | PEX1_HUMAN |
| 591 | GPGPPQLLVSRALLRLLALGSGAWV | PEX6_HUMAN |
| 592 | EFFTHLDKRSLPALTNIIKILRHDI | PH4H_HUMAN |
| 593 | FESIGKFGLALAVAGGVVNSALYNV | PHB_HUMAN |
| 594 | MDRSSKRRQVKPLAASLLEALDYDS | PHFE_HUMAN |
| 595 | TVTWGNYGKSYSVALYLVRQLTSSE | PIA4_HUMAN |
| 596 | TTAKRRLKQSVHLARRVLQLEKQNS | PIBF_HUMAN |
| 597 | RETAEPFLFVDEFLTYLFSRENSIW | PIG2_HUMAN |
| 598 | LVSTLVPLGLVLAVGAVAVGVARAR | PIGR_HUMAN |
| 599 | EVARGKRAALFFAAVAIVLGLPLWW | PIGS_HUMAN |
| 600 | FTSFDQVAQLSSAARGLAASLLFLL | PKD1_HUMAN |
| 601 | GAWARWLLVALTAATALVRLAQLGA | PKD1_HUMAN |
| 602 | LHAAVTLRLEFPAAGRALAALSVRP | PKD1_HUMAN |
| 603 | RMVASQAYNLTSALMRILMRSRVLN | PKD1_HUMAN |
| 604 | ESSTNREKYLKSVLRELVTYLLFLI | PKD2_HUMAN |
| 605 | LLHSRNEGTATYAAAVLFRISEDKN | PLAK_HUMAN |
| 606 | RQFRTGKVTVEKVIKILITIVEEVE | PLE1_HUMAN |
| 607 | RQFRTGRITVEKIIKIIITVVEEQE | PLE1_HUMAN |
| 608 | GPGPRFLLLLPLLLPPAASASDRPR | PLO3_HUMAN |
| 609 | DILKPGGGTSGGLLGGLLGKVTSVI | PLUN_HUMAN |
| 610 | VLRGLDITLVHDIVNMLIHGLQFVI | PLUN_HUMAN |
| 611 | LGQVPLIVGILLVLMAVVLASLIYR | PM17_HUMAN |
| 612 | PDKRQILLQEEKLLLAVLKTSLIGM | PMS2_HUMAN |
| 613 | GLNPSLMAPSQFAAGGALLSLNPGT | PO21_HUMAN |
| 614 | ITLGYTQADVGLILGVLFGKVFSQK | PO57_HUMAN |
| 615 | ITLGYTQADVGLILGVLFGKVFSQT | PO5M_HUMAN |
| 616 | LFSKYTNSKIPYFLLFLIFLITVYH | PP3A_HUMAN |
| 617 | DFVDRGFYSVETFLLLLALKVRYPD | PP4C_HUMAN |
| 618 | LLLARAASLSLGFLFLLFFWLDRSV | PPAP_HUMAN |
| 619 | LVQIIKKTESDAALHPLLQEIYRDM | PPAR_HUMAN |
| 620 | QTWLSALRPSGPALSGLLSLEAEEN | PPCS_HUMAN |
| 621 | SMEGVTFLQAKQIALHALSLVGEKQ | PPO4_HUMAN |
| 622 | VVGTTTTTPSPSAIKAAAKSAALQV | PRCC_HUMAN |
| 623 | NDLTRNRFFENPALWELLFHSIHDA | PRES_HUMAN |
| 624 | PPSGIHLSASRTLAPTLLYSSPPSH | PS11_HUMAN |
| 625 | KRLFMNDRHVGMAVAGLLADARSLA | PSA3_HUMAN |
| 626 | RSNFGYNIPLKHLADRVAMYVHAYT | PSA3_HUMAN |
| 627 | VLYEDEGFRSRQFAALVASKVFYHL | PSD1_HUMAN |
| 628 | HLLRYGEPTLRRAVPLALALISVSN | PSD2_HUMAN |
| 629 | GYGHMWSQNATNLVSSLLTLLKQLE | PTN5_HUMAN |
| 630 | KSLLDPKVAARLAVAEALTNLVFAL | PUR4_HUMAN |
| 631 | AQVGLGVGTSLLALGVIIIVLMYRR | PXB1_HUMAN |
| 632 | TRLLSMKGTLQKFVDDLFQVILSTS | PXB1_HUMAN |
| 633 | LVVPLPFRDLLLVARGLAGKLSAGV | PXB3_HUMAN |
| 634 | GELSARQMHLARFLRMLLRLADEFG | RA51_HUMAN |
| 635 | IDLVSKLLYSRGLLIDLLTKSNVSR | RAE1_HUMAN |
| 636 | IDLVSKLLYSQGLLIDLLIKSDVSR | RAE2_HUMAN |
| 637 | AIFTGHSAVVEDVAWHLLHESLFGS | RBB7_HUMAN |
| 638 | EHPAIRTLSARAAAAFVLANENNIA | RBP6_HUMAN |
| 639 | MARGGRGRRLGLALGLLLALVLAPR | RCN1_HUMAN |
| 640 | FEEYLRALDVNVALRKIANLLKPDK | RET1_HUMAN |
| 641 | MEDYLQALNISLAVRKIALLLKPDK | RET5_HUMAN |
| 642 | HSYVSVKAKVSSIAQEILKVVAEKI | RGE5_HUMAN |
| 643 | RLLWRLPAPVLVVLRYLFTFLNHLA | RHG4_HUMAN |
| 644 | PKPVVPKTNVKALVPNLLRAIEAGI | RHG5_HUMAN |
| 645 | YPRKFNETQIKQALRGVLESVKHNL | RHG5_HUMAN |
| 646 | KKNFESLSEAFSVASAAAVLSHNRY | RIB2_HUMAN |
| 647 | VSTTVAKAMAREAAQRVAESSRLEK | RIP2_HUMAN |
| 648 | DPVLRKKNGATPFILAAIAGSVKLL | RN5A_HUMAN |
| 649 | AHKATNKSSETLALLEILKHIAITE | RP1_HUMAN |
| 650 | LNYVASQPKLAPFVIQALIQVIAKI | RP17_HUMAN |
| 651 | YGDNHFDNVLQAFVKMLLSVSHSDL | RP17_HUMAN |
| 652 | WVSVLLKKTEKAFLAHLASAVAELR | RPL1_HUMAN |
| 653 | FMKLVGMPYLHEVLKPVISRVFEEK | RSG4_HUMAN |
| 654 | QHADPQTSRSLLLLAKAVQSIGNLG | RSG4_HUMAN |
| 655 | INLKRTWEKLLLAARAIVAIENPAD | RSSA_HUMAN |
| 656 | SQGMVGQLAARRAAGVVLEMIREGK | RUV2_HUMAN |
| 657 | EQGKRNFSKAMSVAKQVFNSLTEYI | RYR1_HUMAN |
| 658 | IDEASWMKRLAVFAQPIVSRARPEL | RYR1_HUMAN |
| 659 | NYLSRNFYTLRFLALFLAFAINFIL | RYR1_HUMAN |
| 660 | QAGKGEALRIRAILRSLVPLEDLVG | RYR1_HUMAN |
| 661 | RVRRLRRLTAREAATAVAALLWAAV | RYR1_HUMAN |
| 662 | TAAAGATARVVAAAGRALRGLSYRS | RYR1_HUMAN |
| 663 | VEKSPHEQEIKFFAKILLPLINQYF | RYR1_HUMAN |
| 664 | ARNFYNMRMLALFVAFAINFILLFY | RYR2_HUMAN |
| 665 | DTPSIEKRFAYSPLQQLIRYVDEAH | RYR2_HUMAN |
| 666 | EQGQRNFSKAIQVAKQVFNTLTEYI | RYR2_HUMAN |
| 667 | GEHFPVEQEIKPFAKVVLPLIDQYF | RYR2_HUMAN |
| 668 | EESGMAWKEILNLLYKLLAALIRGN | RYR3_HUMAN |
| 669 | HYLARNFYNLRFLALFVAFAINFIL | RYR3_HUMAN |
| 670 | QTGKGEAIRIRSILRSLVPTEDLVG | RYR3_HUMAN |
| 671 | TEKSPRDQEIKFFAKVLLPLVDQYF | RYR3_HUMAN |
| 672 | TLYQQARLHERGAAEMVLQMISASK | RYR3_HUMAN |
| 673 | FLSGQGLAGIFAALAMLLSMASGVD | S292_HUMAN |
| 674 | SNSLAYYNMANGAVIHLALKERGGR | S3A1_HUMAN |
| 675 | LQLLSGHPPASEAVASVLSFLYDKK | S3T2_HUMAN |
| 676 | QISLEGYEKALEFATLAARLSTVTG | S3T2_HUMAN |
| 677 | HTSTLAAMKLMTALVNVALNLSINM | SA2_HUMAN |
| 678 | APPVAAGVGAVLAAGALLGLVAGAL | SBN1_HUMAN |
| 679 | DMVKSKWGLALAAVVTVLSSLLMSV | SCAP_HUMAN |
| 680 | GMTWSHGLSVSKVLHKAFVEVTEEG | SCC2_HUMAN |
| 681 | AGKSGGSAGEITFLEALARSESKRD | SEN6_HUMAN |
| 682 | ILQKYIERIITRFAPMLVPYTWQNQ | SGT1_HUMAN |
| 683 | DAQLDYHRQAVQILDELAEKLKRRM | SH31_HUMAN |
| 684 | FKKDPPLAAVTTAVQELLRLAQAHP | SKIW_HUMAN |
| 685 | RGLGVHHSGILPILKEIVEMLFSRG | SKIW_HUMAN |
| 686 | RRIDLSNNQIAEIAPDAFQGLRSLN | SLT1_HUMAN |
| 687 | SSAGPVRPELWLLLWAAAWRLGASA | SLT1_HUMAN |
| 688 | VTVLFALVLSGALIILVASPLRALR | SM4A_HUMAN |
| 689 | PGGMNRKTQETAVAMHVAANSIQNR | SMF1_HUMAN |
| 690 | TSTWLDDVEERLFVATALLPEETET | SNE1_HUMAN |
| 691 | INSQLARHTSPSVISDLFTDIKKGH | SNE2_HUMAN |
| 692 | QHVDQRRQGLEDFLRKVLQNALLLS | SNXA_HUMAN |
| 693 | PDIPEWRKDIGNVIKRALVKVTSVP | SOR3_HUMAN |
| 694 | LQHRHRLPDLQAILRRILNEEETSP | SP90_HUMAN |
| 695 | HNNRRLQAESESAATRLLLASKQLG | SPA1_HUMAN |
| 696 | RAAPRGPGAELQAAGSLVWGVRAAP | SPA1_HUMAN |
| 697 | RNVFFSPMSISSALAMVFMGAKGST | SPB8_HUMAN |
| 698 | QLAKQKAQEAEKLLNNVISKLLPTN | SPC2_HUMAN |
| 699 | SLLDKHSQIINKFVNSVINTLKSTV | SPC2_HUMAN |
| 700 | IQSLTMYPRLGGFVMNILSRLIMKQ | SPK_HUMAN |
| 701 | VHPAISSINLTTALGSLANIARQRP | SPK_HUMAN |
| 702 | PFYDPEGGSITQVARVVIERIARKG | SPO1_HUMAN |
| 703 | PLKLSRTPALLALALPLAAALAFSD | SPO1_HUMAN |
| 704 | MVGPAPRRRLRPLAALALVLALAPG | SPUF_HUMAN |
| 705 | KTGSFKIRGALNAVRSLVPDALERK | SRR_HUMAN |
| 706 | KHGPGRWVVLAAVLIGLLLVLLGIG | ST14_HUMAN |
| 707 | SNRGLTKENLVFLAQKLFNNSSSHL | ST5B_HUMAN |
| 708 | DASKALLGRLTTLIELLLPKLEEWK | STA2_HUMAN |
| 709 | KGVDLRNAQVTELLQRLLHRAFVVE | STA2_HUMAN |
| 710 | PAAGLGPGHARHVLRSLVNQSVQDG | STRC_HUMAN |
| 711 | PDNTGRGYVLRRILRRAVRYAHEKL | SYA_HUMAN |
| 712 | RRPIMSNHTATHILNFALRSVLGEA | SYA_HUMAN |
| 713 | FLAGETESLADIVLWGALYPLLQDP | SYM_HUMAN |
| 714 | YHQLLEKVRIRDALRSILTISRHGN | SYM_HUMAN |
| 715 | FLKGVLVFLEQALIQYALRTLGSRG | SYS_HUMAN |
| 716 | WEVGVYAAGALALLGIAAVSLWKLW | SYTC_HUMAN |
| 717 | WLYLQDQNKAADAVGEILLSLSYLP | SYTC_HUMAN |
| 718 | TRPWLLDPKTLKFVVFIVAVLLPVR | T10D_HUMAN |
| 719 | TWKDRFPGYLMNFASILFMIALTFS | T16B_HUMAN |
| 720 | EVVKLHPHELNNLLSKVLIYLRSAN | T172_HUMAN |
| 721 | YALAVRQDVINTLLPKVLTRIIEGL | T172_HUMAN |
| 722 | MADPDVLTEVPAALKRLAKYVIRGF | T2EA_HUMAN |
| 723 | FSQGKMYGYVDTLLTMLAMLLKVAM | T3C3_HUMAN |
| 724 | KLQEIMMHVIWAALAFAAIQLLGML | T4S8_HUMAN |
| 725 | DTEFAKQTSLDAVAQAVVDRVKRIH | TAB1_HUMAN |
| 726 | LEFAIMRIEALKLARQIALASRSHQ | TAC2_HUMAN |
| 727 | RANTHIRDFLQVFIYRLFWKSKDRP | TAF1_HUMAN |
| 728 | DIERPTYTNLNRLTSQIVSSITASL | TBA8_HUMAN |
| 729 | QTIIAGWREATKAAREALLSSAVDH | TCPB_HUMAN |
| 730 | LDTYLGKYWAIKLATNAAVTVLRVD | TCPQ_HUMAN |
| 731 | NFGAFSINPAMMAAAQAALQSSWGM | TDBP_HUMAN |
| 732 | QKLYIPRSTATAALGAAARLATSRS | TDR5_HUMAN |
| 733 | EEEEKVSQPEVGAAIKIIRQLMEKF | TE2I_HUMAN |
| 734 | LSKKLIYFQLHRALKMIVDPVEPHG | TF1B_HUMAN |
| 735 | GSRGTTAGSSGDALGKALASIYSPD | TFE2_HUMAN |
| 736 | LPSIPAQPISADIASRLLRKLKGPV | TFR2_HUMAN |
| 737 | NLHKVLQGRLPAVAQAVAQLAGQLL | TFR2_HUMAN |
| 738 | QRDAWGPGAAKSAVGTAILLELVRT | TFR2_HUMAN |
| 739 | EAFSHFTKIITPAITRVVDFAKKLP | THB2_HUMAN |
| 740 | EGQSQQFSVSENLLKEAIRAIFPSR | THYG_HUMAN |
| 741 | PASLGKWKKEPELAAFVFKTAVVLV | TIAM_HUMAN |
| 742 | RLPSSWALFSPLLAGLALLGVGPVP | TIP_HUMAN |
| 743 | AIKKKLVQRLEHAAKQAAASATQTI | TLN1_HUMAN |
| 744 | GQLLRGVGAAATAVTQALNELLQHV | TLN1_HUMAN |
| 745 | QQLAAFSKRVAGAVTELIQAAEAMK | TLN2_HUMAN |
| 746 | SELLKQVSAAASVVSQALHDLLQHV | TLN2_HUMAN |
| 747 | KVLNLAYNKINKIADEAFYGLDNLQ | TLR5_HUMAN |
| 748 | GREKFKSRGVGELARLALVISELEG | TM26_HUMAN |
| 749 | QGHQFLREREEHLLEQLAKLEQELT | TM26_HUMAN |
| 750 | LKISPVAPDADAVAAQILSLLPLKF | TMS3_HUMAN |
| 751 | RKARGYLRLVPLFVLLALLVLASAG | TMS6_HUMAN |
| 752 | YAHPQQKVAVYRALQAALAESGGSP | TRAD_HUMAN |
| 753 | YVNYVLSEKSSTFLMKAAAKVVESK | TRF1_HUMAN |
| 754 | AAPAPGAPLLPLLLPALAARLLPPA | TRFM_HUMAN |
| 755 | THIKAPEQQVKNILNELFQRENRVL | TRIO_HUMAN |
| 756 | PHMRKNIKGIHTLLQNLAKASPVYL | TRKB_HUMAN |
| 757 | QHALRNRRLLRKVIKAAALEEHNAA | TSC1_HUMAN |
| 758 | KSDRLISLQSASVVYDLLTIALGRR | TT7B_HUMAN |
| 759 | QMKAKRTKEAVEVLKKALDAISHSD | TTC6_HUMAN |
| 760 | LARQINHPELHMVLSNLAAVLMHRE | TTCJ_HUMAN |
| 761 | VAPHGRGPGLLPLLAALAWFSRFAA | TTCJ_HUMAN |
| 762 | HSRTSWVPVVLGVLTALVTAAALAL | TYO3_HUMAN |
| 763 | PLPLPPPPRLGLLLAALASLLLPES | TYO3_HUMAN |
| 764 | WSWLLGAAMVGAVLTALLAGLVSLL | TYRO_HUMAN |
| 765 | LLHDDRGPVLEALVARAIRNIEMTQ | U520_HUMAN |
| 766 | RGGGQIIPTARRVVYSAFLMATPRL | U5S1_HUMAN |
| 767 | LFYQDKLKSLHQLLEVLLALLDKDV | UB24_HUMAN |
| 768 | YGSGPKRFPLVDVLQYALEFASSKP | UB25_HUMAN |
| 769 | NRKPVDPSAALDLLKGAFRSSEEQQ | UB28_HUMAN |
| 770 | KIEKVFSKLLYPIVRGAALSVLKYM | UB35_HUMAN |
| 771 | RFLNLLMNDAIFLLDEAIQYLSKIK | UB4A_HUMAN |
| 772 | DIPSADRHKSKLIAGKIIPAIATTT | UBA1_HUMAN |
| 773 | GIPPVNRAQSKRIVGQTIPAIATTT | UBAL_HUMAN |
| 774 | PVDKVAAMREFRVLHTALHSSSSYR | ULSB_HUMAN |
| 775 | AARFAKTKEEVEAAKAAALLAKQAE | UXD2_HUMAN |
| 776 | HLPEKQDTFAEKLVTQIIKNLQVKI | V13A_HUMAN |
| 777 | MLNRQDPFTVHMAARIIAKLAAWGK | VATH_HUMAN |
| 778 | ALIEGKNKTVSTLVIQAANVSALYK | VGR2_HUMAN |
| 779 | EVITDNLPGSIRAVVNIFLVAKALL | VIAA_HUMAN |
| 780 | VRWLQESRRSRKLILFIVFLALLLD | VMT2_HUMAN |
| 781 | FVIATTRQRLFQFIGRAAEGAEAQG | VP18_HUMAN |
| 782 | WIEMGSRLDARQLIPALVNYSQGGE | VP18_HUMAN |
| 783 | MRDVNKKFSVRYFLNLVLVDEEDRR | VP26_HUMAN |
| 784 | NSFKMKMSVILGIIHMLFGVSLSLF | VPP1_HUMAN |
| 785 | NSYKMKMSVILGIVQMVFGVILSLF | VPP4_HUMAN |
| 786 | MQREGGPSQIGDALDFAVRYLTSEM | VWF_HUMAN |
| 787 | KDTPSGISKVRKILFTLAKQSKALG | WD10_HUMAN |
| 788 | LVGLKNGQILKIFVDNLFAIVLLKQ | WD10_HUMAN |
| 789 | SFEQGGSEFVPVVVSRLVLRSMSRR | WD10_HUMAN |
| 790 | IPADPEAGGIGRVVNGAFMVLKGHR | WD22_HUMAN |
| 791 | RQNKDERYSIKDLLNHAFFQEETGV | WNK1_HUMAN |
| 792 | RKTSKSKLKAGKLLNPLVRQLKVVA | WNK2_HUMAN |
| 793 | RTDKNERFTIQDLLAHAFFREERGV | WNK4_HUMAN |
| 794 | TAFGADTEGSQWIIGYLLWKVISNL | XPO4_HUMAN |
| 795 | LGLNDETMVLSVFIGKIITNLKYWG | XPO7_HUMAN |
| 796 | LNYLATRPKLATFVTQALIQLYARI | XPO7_HUMAN |
| 797 | AMFVEYRKQLKLLLDRLAQVSPELL | XPOT_HUMAN |
| 798 | QNWQTTRFMEVEVAIRLLYMLAEAL | XPOT_HUMAN |
| 799 | FPAATSGRMVEAFARRALWDAGLNY | XPP2_HUMAN |
| 800 | LLKASHVRDAVAVIRYLVWLEKNVP | XPP2_HUMAN |
| 801 | SILTQPHLYSPVLISQLVQMASQLR | Y310_HUMAN |
| 802 | ISIGTIYFRAHKLVLAAASLLFKTL | Y478_HUMAN |
| 803 | RDVLRVQGVSLTALRLLLADAYSGR | Y711_HUMAN |
| 804 | QLEGLENATARNLLGKLINILLAVM | Y779_HUMAN |
| 805 | PPLIVDRERLKKLLDLLVDKSNNLA | YC40_HUMAN |
| 806 | EDTKEKRTIIHQAIKSLFPGLETKT | YI97_HUMAN |
| 807 | DQKTNLPEYLQTLLNTLAPLLLFRA | Z294_HUMAN |
| 808 | RSREHGTLWSLIIAKLILSRSISSD | Z294_HUMAN |
| 809 | VLASAYTGRLSMAAADIVNFLTVGS | Z297_HUMAN |
| 810 | GRNKMDPPRSSIFLQEVITTVYGYK | ZAN_HUMAN |
| 811 | VDLAYSNYHVKQFLEALLRNSAAPS | ZBT8_HUMAN |
| 812 | LKLRSLRVNVGQLLAMIVPDLDLQQ | ZCW3_HUMAN |
| 813 | ELQKQAELMEFEIALKALSVLRYIT | ZM10_HUMAN |
| 814 | DALHMLTDLSAIILTLLALWLSSKS | ZNT4_HUMAN |
| 815 | MDDEENYSAASKAVRQVLHQLKRLG | ZW10_HUMAN |

### LEGENDS TO THE FIGURES

FIG. 1:

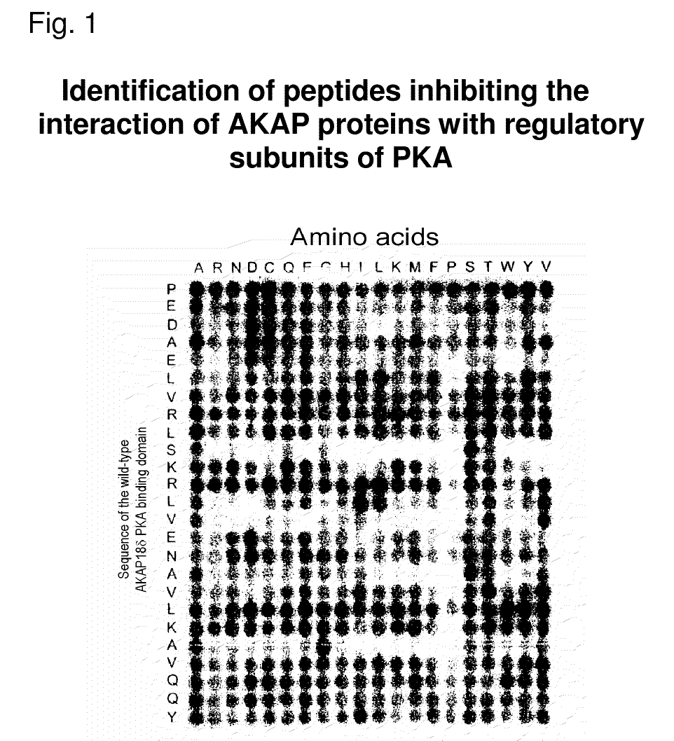

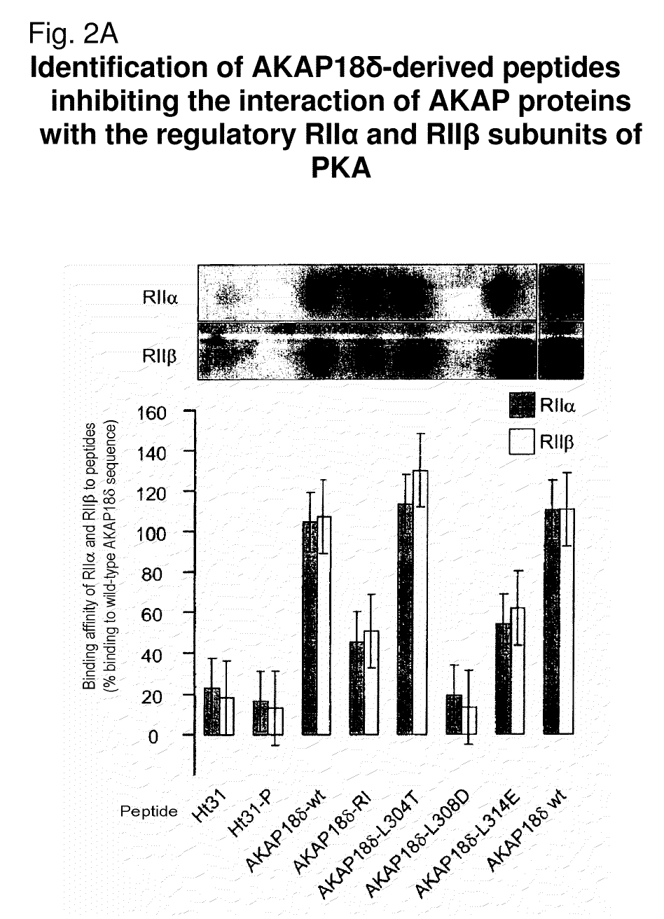

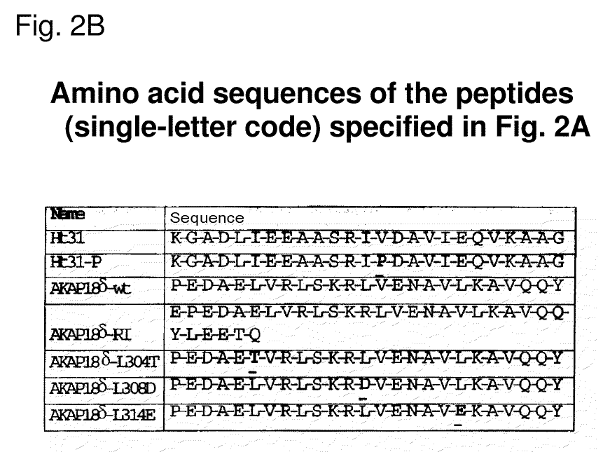

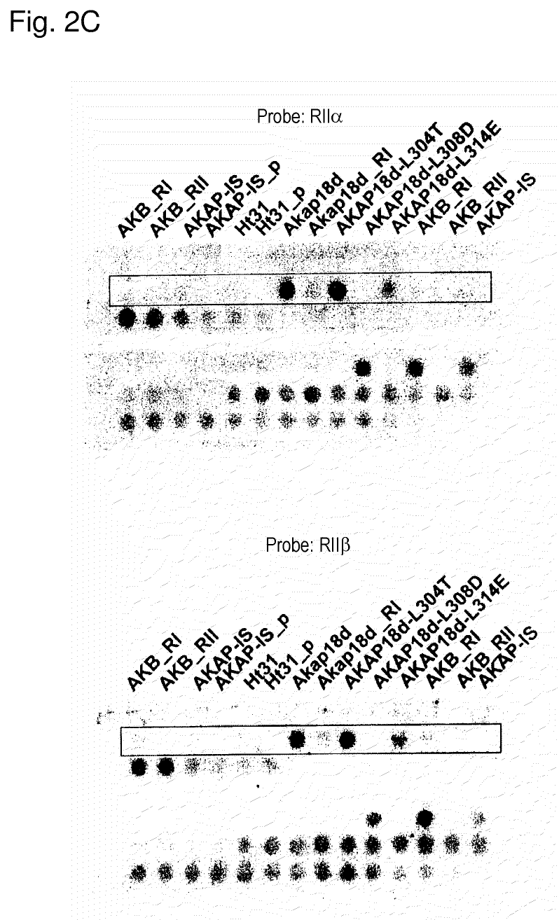

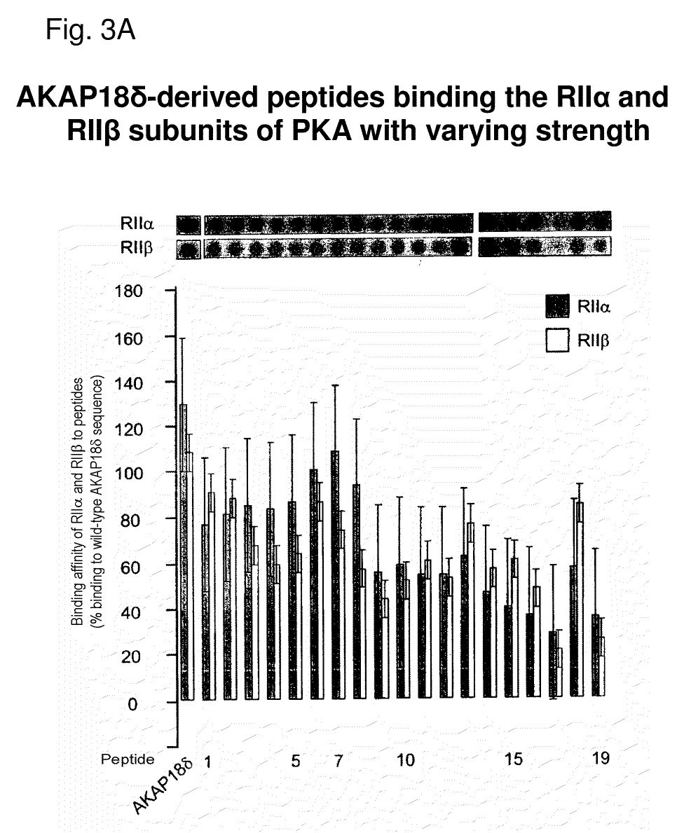

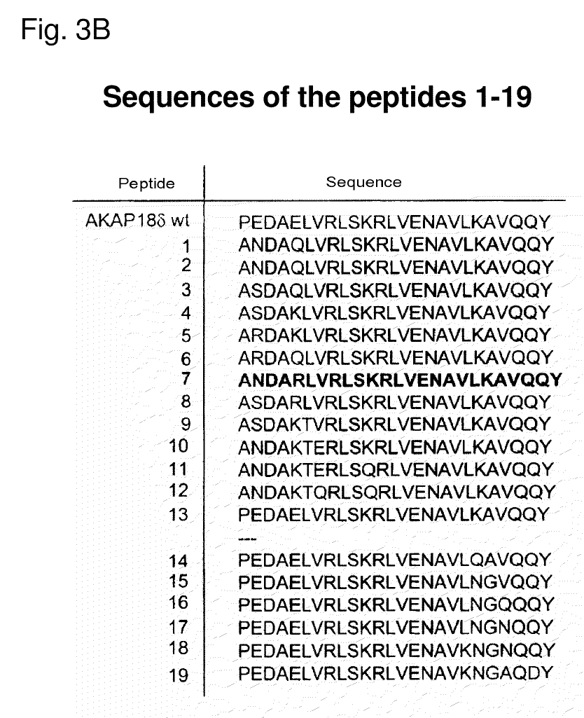

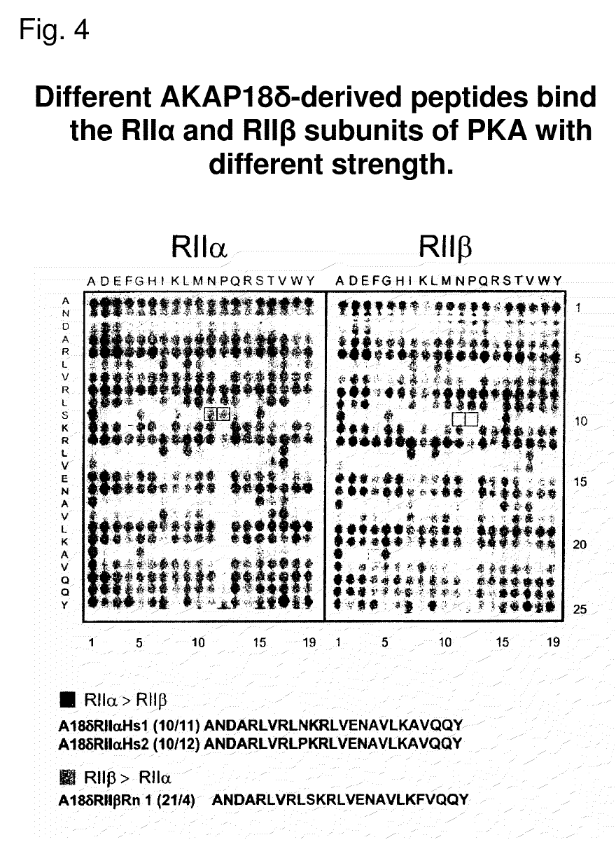

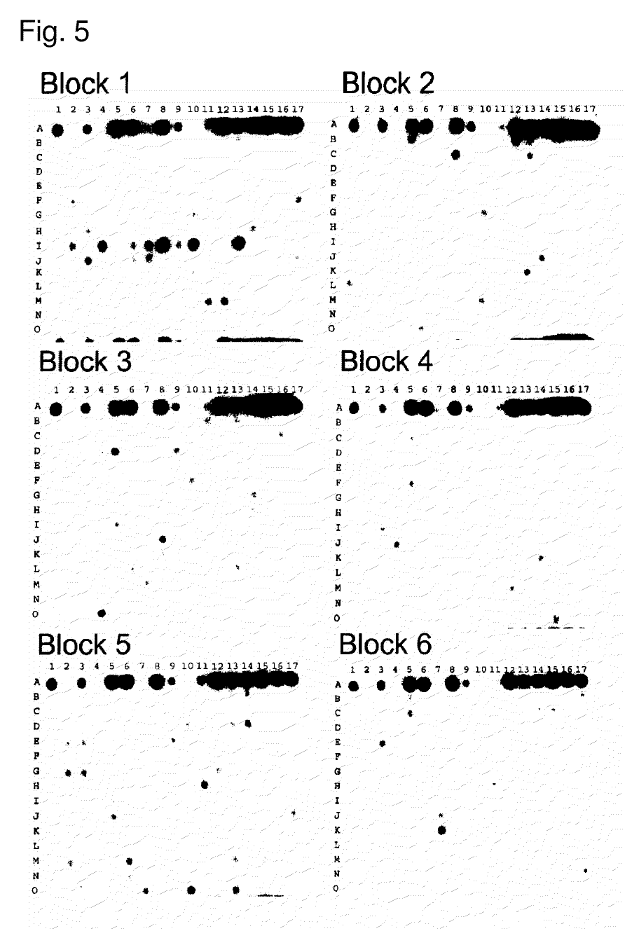

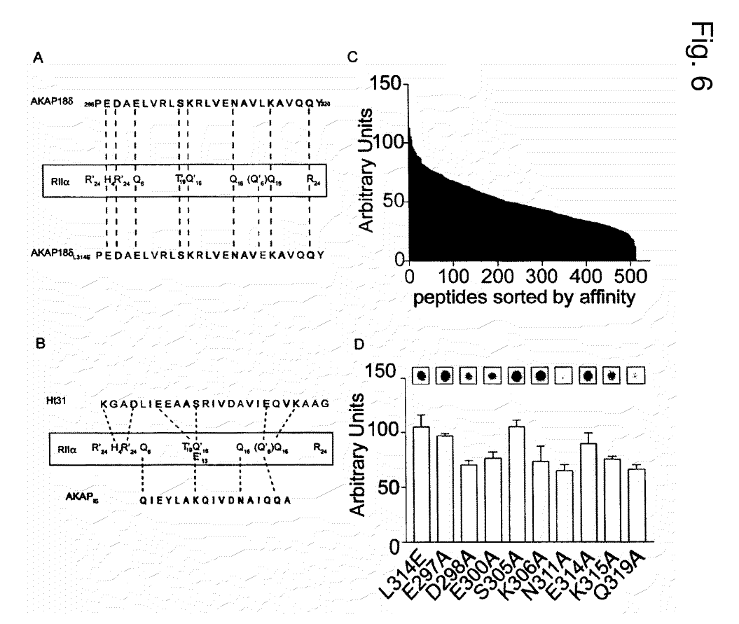

Identification of peptides inhibiting the interaction of AKAP proteins with PKA. A library of peptides derived from the PKA binding domain of AKAP18δ was synthesized on a membrane. The membrane was incubated with radiolabelled regulatory RIIα and RIIβ subunits of PKA (RII overlay experiment). Each black dot represents a peptide having bound the RII subunits thereto (detected using a phosphoimager). The amino acid sequences of the peptides can be read with the help of the abbreviations as specified (single-letter code).
Vertical: Sequence of the wild-type PKA binding domain of AKAP18δ.
Horizontal: the 20 amino acids used in the substitution of the wild-type sequence.

FIG. 2:

Identification of AKAP18δ-derived peptides inhibiting the interaction of AKAP proteins with the regulatory RIIα and RIIβ subunits of PKA. A: Peptides derived from the PKA binding domain of AKAP18δ were synthesized on two membranes. The membranes were incubated with radiolabelled regulatory RIIα (upper row) or RIIβ subunits (row below) of the PKA (RII overlay experiment). Each black dot represents a peptide having bound the RII subunits thereto (detected using a phosphoimager). For quantification, the signals were evaluated by means of densitometry and correlated with the signal obtained for AKAP18δ-wt. B: The amino acid sequences of the peptides (single-letter code) specified in A.

FIG. 3:

AKAP18δ-derived peptides binding the RIIα and RIIβ subunits of PKA with varying strength. A: The peptides 1-19 derived from the PKA binding domain of AKAP18δ were synthesized on two membranes. The membranes were incubated with radio-labelled regulatory RIIα (upper row) or RIIβ subunits (row below) of the PKA (RII overlay experiment). Each black dot represents a peptide having bound the RII subunits thereto (detected using a phosphoimager). For quantification, the signals were evaluated by means of densitometry and correlated with the signal obtained for AKAP18δ-wt. Owing to the great difference in binding to both RII subunits, peptide No. 7 is highlighted in red printing. B: The amino acid sequences of the peptides (single-letter code) specified with 1-19 in A.

FIG. 4:

Different AKAP18δ-derived peptides bind the RIIα and RIIβ subunits of PKA with different strength. Two libraries of peptides derived from peptide 7 of FIG. 3 were synthesized on two membranes. The membranes were incubated with radiolabelled regulatory RIIα (left) or RIIβ subunits (right) of the PKA (RII overlay experiment). Each black dot represents a peptide having bound the RII subunits thereto (detected using a phosphoimager). The amino acid sequences of the peptides can be read with the help of the abbreviations as specified (single-letter code). Vertical: Sequence of peptide 7; horizontal: the 20 amino acids used in the substitution of the wild-type sequence. The horizontal and vertical rows are additionally labeled with Arabic numerals. These coordinates facilitate the assignment. Thus, for example, 10/11 means: row 10, peptide 11. The peptides listed below are denoted A18δRIIα Hs1 and 2 in accordance with their binding to RIIα and A18δRIIβRn1 in accordance with their binding to RIIβ.

FIG. 5:

Identification of peptides inhibiting the AKAP-PKA interactions. Candidate peptides were synthesized on a membrane and incubated with radiolabelled regulatory RII□ subunits of PKA (RII overlay experiment). All black dots represent peptides having bound regulatory PKA subunits (detected using a phosphoimager).

FIG. 6:

Influence of hydrogen bridges on binding between peptides and RIIα subunits of PKA. (A, B): Comparative schematic representation of the interaction between RIIα and the peptides AKAP18δ-wt or AKAP18δ-L314E and between RIIα, Ht31 or AKAP_{IS}. RIIα is represented as a rectangle and by selected amino acids, the peptides are represented with the help of their amino acid sequence. Amino acids as participants of a hydrogen bridge are linked by a broken line. Amino acids of peptides located in positions for hydrophobic molecular contacts are highlighted in green (position of amino acids of AKAP18δ-wt given in comparison to the protein). (C, D): To investigate the influence of the amino acids on the binding strength, alanine-substituted peptides were synthesized on membranes, checked for RIIα binding by means of RII overlay and quantified using densitometry. Starting from AKAP18δ-L314E, the peptides were substituted in all possible combinations with amino acids capable of forming hydrogen bridges (see A). The quantification for all peptides, sorted by affinity, is illustrated in C. The quantification for all single substitutions (as specified), as well as representative “spots” from an RII overlay (top) are illustrated in D.

### REFERENCES

Alto, N. M., Soderling, S. H., Hoshi, N., Langeberg, L. K., Fayos, R., Jennings, P. A., Scott, J. D., Bioinformatic design of A kinase-anchoring protein in silico: a potent and selective peptide antagonist of type II protein kinase A anchoring. Proc. Natl. Acad. Sci. USA 100, 4445-4450, 2003.
Bregman, D. B., Bhattacharyya, N., Rubin, C. S. High-affinity binding protein for the regulatory subunit of cAMP-dependent protein kinase II-B. J. Biol. Chem. 264, 4648-4656, 1989.
Burns-Hamuro, L. L., Ma, Y., Kammerer, S., Reineke, U., Self, C., Cook, C., Olson, G. L., Cantor, C. R., Braun, A., Taylor, S. S., Designing isoform-specific peptide disruptors of protein kinase A localization. Proc. Natl. Acad. Sci. USA 100, 4072-4077, 2003.
Frank, R. Spot synthesis: an easy technique for the positionally addressable, parallel chemical synthesis on a membrane support. Tetrahedron 48, 9217-9232, 1992.
Fräser, I. D., Tavalin, S. J., Lester, L. B., Langeberg, L. K., Westphal, A. M., Dean, R. A., Marrion, N. V., Scott, J. D., A novel lipid-anchored A kinase anchoring protein facilitates cAMP-responsive membrane events. EMBO J. 17, 2261-2272, 1998.
Henn, V., Edemir, B., Stefan, E., Wiesner, B., Lorenz, D., Theilig, F., Schmitt, R., Vossebein, L., Tamma, G., Beyermann, M., Krause, E., Herberg. F. W., Valenti, G., Bachmann, S., Rosenthal, W., Klussmann, E., Identification of a novel A kinase anchoring protein 18 isoform and evidence for its role in the vasopressin-induced aquaporin-2 shuttle in renal principal cells. J. Biol. Chem. JBC, published Mar. 22, 2004 as doi:10.1074/jbc.M312835200.
Hulme J. T., Lin, T. W., Westenbroek, R. E., Scheuer, T., Catterall, W. A., B-adrenergic regulation requires direct anchoring of PKA to cardiac CaVl.2 channels via a leucine zipper interaction with A kinase-anchoring protein 15. Proc. Natl. Acad. Sci. USA 100, 13093-13098, 2003.
Klussmann, E., Marie, K., Wiesner, B., Beyermann, M., Rosenthal, W., Protein kinase A anchoring proteins are required for vasopressin-mediated translocation of aquaporin-2 into cell membranes of renal principal cells. J. Biol. Chem. 274, 4934-4938, 1999.
Klussmann, E., Protein kinase A. Online pharmacology reference database. Elsevier Science Inc., Amsterdam, The Netherlands. In press.
Kramer, A., Schneider-Mergener, J., Synthesis and screening of peptide libraries on continuous cellulose membrane supports. Meth. Mol. Biol. 87, 25-39, 1998.
Tasken, K., Aandahl, E. M., Localized effects of cAMP mediated by distinct routes of protein kinase A. Physiol. Rev. 84, 137-167, 2004.

1. Protein kinase A/protein kinase A anchor protein decouplers, wherein the decouplers are derived from either (i) an AKAP18δ or (ii) a protein other than AKAP18δ and, according to (i), have amino acids forming at least 8H bridges, or, according to (ii), have the general formula (1):
xxxxxxxxx[AVLISE]xx[AVLIF][AVLI]xx[AVLI][AVLIF]xx
[AVLISE]xxxx (1),
wherein x can be any of 20 biogenic amino acids.

2. An isolated nucleic acid molecule selected from the group comprising:
a) a nucleic acid molecule comprising a nucleotide sequence encoding at least one amino acid sequence according to SEQ ID Nos. 1-39,
b) a nucleic acid molecule which undergoes hybridization with a nucleotide sequence according to a) under stringent conditions,
c) a nucleic acid molecule comprising a nucleotide sequence having sufficient homology to be functionally analogous to a nucleotide sequence according to a) or b),
d) a nucleic acid molecule which, as a consequence of the genetic code, is degenerated into a nucleotide sequence according to a)-c), and
e) a nucleic acid molecule in accordance with a nucleotide sequence according to a)-d), which is modified and functionally analogous to a nucleotide sequence according to a)-d) as a result of deletions, additions, substitutions, translocations, inversions and/or insertions.

3. The nucleic acid molecule according to claim 2, wherein the nucleotide sequence specified under c) has at least 60%, preferably 70%, more preferably 80%, especially preferably 90% homology to a nucleotide sequence as specified under a).

4. The nucleic acid molecule according to claim 2 wherein said molecule is a genomic DNA, a cDNA and/or an RNA.

5. A vector comprising a nucleic acid molecule according to claim 2.

6. A host cell comprising the vector according to claim 5.

7. An organism comprising a nucleic acid molecule according to claim 2, wherein said nucleic acid is optionally part of a vector comprising said nucleic acid or a host cell comprising such a vector.

8. The organism according to claim 7, wherein
the organism is a transgenic mouse or rat, said mouse or rat developing insipid diabetes preferably as a result of the presence of the nucleic acid molecule, the vector or the host cell.

9. A polypeptide encoded by a nucleic acid molecule according to claim 2.

10. The polypeptide according to claim 9, wherein
a) the polypeptide comprises an amino acid sequence according to SEQ ID 1 to 39,
b) the polypeptide according to a) has been modified by deletions, additions, substitutions, translocations, inversions and/or insertions and is functionally analogous to a polypeptide according to a), and/or
c) the polypeptide comprises a polypeptide which has sufficient homology to be functionally analogous to a polypeptide according to a) or b).

11. A recognition molecule directed against a nucleic acid molecule according to claim 2, wherein said nucleic acid is optionally part of a vector comprising said nucleic acid or a host cell comprising such a vector a vector, a protein kinase A/protein kinase A anchor protein decoupler, wherein the decouplers are derived from either (i) an AKAP18δ or (ii) a protein other than AKAP18δ and, according to (i), have amino acids forming at least 8H bridges, or, according to (ii), have the general formula (1):
xxxxxxxxx[AVLISE]xx[AVLIF][AVLI]xx[AVLI][AVLIF]xx
[AVLISE]xxxx (1),
wherein x can be any of 20 biogenic amino acids and/or
a polypeptide
wherein
a) the polypeptide comprises an amino acid sequence according to SEQ ID 1 to 39,
b) the polypeptide according to a) has been modified by deletions, additions substitutions, translocations, inversions and/or insertions and is functionally analogous to a polypeptide according to a), and/or
c) the polypeptide comprises a polypeptide which has sufficient homology to be functionally analogous to a polypeptide according to a) or b).

12. The recognition molecule according to claim 11, wherein
said molecule is an antibody, an antibody fragment and/or an antisense construct, particularly an RNA interference molecule.

13. A pharmaceutical composition,
wherein
said composition comprises
a) a nucleic acid molecule comprising a nucleotide sequence encoding at least one amino acid sequence according to SEQ ID Nos. 1-39,
b) a nucleic acid molecule which undergoes hybridization with a nucleotide sequence according to a) under stringent conditions,
c) a nucleic acid molecule comprising a nucleotide sequence having sufficient homology to be functionally analogous to a nucleotide sequence according to a) or b),
d) a nucleic acid molecule which, as a consequence of the genetic code, is degenerated into a nucleotide sequence according to a)-c), and
e) a nucleic acid molecule in accordance with a nucleotide sequence according to a)-d), which is modified and functionally analogous to a nucleotide sequence according to a)-d) as a result of deletions, additions, substitutions, translocations, inversions and/or insertions, wherein said nucleic acid is optionally part of a vector comprising said nucleic acid or a host cell comprising such a vector, a polypeptide wherein
a) the polypeptide comprises an amino acid sequence according to SEQ ID 1 to 39,
b) the polypeptide according to a) has been modified by deletions, additions substitutions, translocations, inversions and/or insertions and is functionally analogous to a polypeptide according to a), and/or
c) the polypeptide comprises a polypeptide which has sufficient homology to be functionally analogous to a polypeptide according to a) or b) and/or a recognition molecule according to claim 11, optionally together with a pharmaceutically tolerable carrier.

14. The pharmaceutical composition according to claim 13, wherein the composition is an aquaretic agent.

15. A kit,
wherein
said kit comprises (i)
a) a nucleic acid molecule comprising a nucleotide sequence encoding at least one amino acid sequence according to SEQ ID Nos. 1-39,
b) a nucleic acid molecule which undergoes hybridization with a nucleotide sequence according to a) under stringent conditions,
c) a nucleic acid molecule comprising a nucleotide sequence having sufficient homology to be functionally analogous to a nucleotide sequence according to a) or b),
d) a nucleic acid molecule which, as a consequence of the genetic code, is degenerated into a nucleotide sequence according to a)-c), and
e) a nucleic acid molecule in accordance with a nucleotide sequence according to a)-d), which is modified and functionally analogous to a nucleotide sequence according to a)-d) as a result of deletions, additions, substitutions, translocations, inversions and/or insertions, wherein said nucleic acid is optionally part of a vector comprising said nucleic acid or a host cell comprising such a vector,
(ii) a polypeptide,
wherein
a) the polypeptide comprises an amino acid sequence according to SEQ ID 1 to 39,
b) the polypeptide according to a) has been modified by deletions, additions substitutions, translocations, inversions and/or insertions and is functionally analogous to a polypeptide according to a), and/or
c) the polypeptide comprises a polypeptide which has sufficient homology to be functionally analogous to a polypeptide according to a) or b)
(iii) a recognition molecule according to claim 11 or the pharmaceutical composition comprising (a), (b) or (c), optionally together with a pharmaceutically tolerable carrier.

16. A method for the modification of an AKAP-PKA interaction, comprising:
providing
a) a nucleic acid molecule comprising a nucleotide sequence encoding at least one amino acid sequence according to SEQ ID Nos. 1-39,
b) a nucleic acid molecule which undergoes hybridization with a nucleotide sequence according to a) under stringent conditions,
c) a nucleic acid molecule comprising a nucleotide sequence having sufficient homology to be functionally analogous to a nucleotide sequence according to a) or b),
d) a nucleic acid molecule which, as a consequence of the genetic code, is degenerated into a nucleotide sequence according to a)-c), and
e) a nucleic acid molecule in accordance with a nucleotide sequence according to a)-d), which is modified and functionally analogous to a nucleotide sequence according to a)-d) as a result of deletions, additions, substitutions, translocations, inversions and/or insertions, wherein said nucleic acid is optionally part of a vector comprising said nucleic acid or a host cell comprising such a vector, wherein said nucleic acid is optionally part of a vector comprising said nucleic acid or a host cell comprising such a vector or a polypeptide according to claim 10, and
contacting at least one of said nucleic acids, vectors or polypeptides with a cell, a cell culture, a tissue and/or a target organism.

17. The method according to claim 16,
wherein
the modification is effected on a regulatory RII subunit of the PKA.

18. The method according to claim 17,
wherein
the RII subunits are RIIα and/or RIIβ subunits.

19-25. (canceled)

26. The method according to claim 16, wherein said modification is an inhibition.

27. The method of claim 16, wherein the AKAP-PKA interaction is effected in a cell, a cell culture, a tissue und/or a target organism.

28. The method of claim 16,
wherein the vasopressin-induced redistribution of AQPII is modified, especially prevented.

29. The method of claim 16,
wherein the interaction of the RIIα or RIIβ subunits of PKA with AKAP is modified, especially inhibited.

30. The method of claim 29, wherein the subunits are of human or murine origin.
